(19) United States
(12) Patent Application Publication
(10) Pub. No.: US 2022/0241229 A1
(43) Pub. Date: Aug. 4, 2022

(54) Methods and Compositions for Altering Senescence Associated Secretory Phenotype

(71) Applicant: BUCK INSTITUTE FOR RESEARCH ON AGING, Novato, CA (US)

(72) Inventors: Brian KENNEDY, Novato, CA (US); Gordon LITHGOW, Novato, CA (US); Azar SHAHMIRZADI, Novato, CA (US); Christopher WILEY, Sonoma, CA (US)

(21) Appl. No.: 17/617,436

(22) PCT Filed: Jun. 10, 2020
(86) PCT No.: PCT/US2020/036974

§ 371 Date: Dec. 8, 2021

Related U.S. Application Data
(60) Provisional application No. 62/859,623, filed on Jun. 10, 2019.

(51) Int. Cl. A61K 31/194; A61K 31/07; A61K 31/593; A61K 33/06; A61K 45/06; A61P 9/00; A61P 17/00; A61P 21/00

(52) U.S. Cl. CPC A61K 31/194; A61K 31/07; A61K 31/593; A61P 21/00; A61K 45/06; A61P 9/00; A61P 17/00; A61K 33/06

(57) ABSTRACT

Disclosed herein are methods and compositions comprising alpha ketoglutarate (AKG) for altering senescence-associated secretory phenotype (SASP), and related diseases.

### BACKGROUND

Cellular senescence is viewed as an irreversible cell-cycle arrest mechanism that acts to protect against cancer, but now is also known to play a role in complex biological processes such as development, tissue repair, ageing and age-related disorders. Pharmacological treatment and prevention of the natural declines of age and aging-related diseases has presented challenges to the medical community in part because of the stringent characteristics required of pharmacological agents for this purpose. Aging patient populations impose unusually high burdens on pharmacological therapies to be bioavailable, nontoxic, and lacking long-term adverse effects. Prevention requires treatment of patients which may be at risk to aging-related disorders but experiencing no or mild symptoms, requiring agents that do not damage existing health. Treatment of patients with existing age-related disease requires a high requirement for nontoxicity, so as to not exacerbate existing health conditions. Moreover, treatment or prevention of aging-related disorders in general likely requires treatment with pharmacological agents for many years, thus requiring such agents to lack cumulative toxicity or long-term deleterious effects on organ systems.

Accordingly, there is a need for discovery of agents and combinations of agents that are nontoxic and suitable for administration to subjects before age related disorders are apparent or acute, or for the longer-term administration.

The primary and secondary metabolites present in dietary fruits and vegetables are a chemically diverse pharmacopeia which meets the criteria for treatment of aging related diseases. Due to their longstanding presence in the human diet, many of these compounds have very low toxicity and well-understood pharmacokinetic parameters. Screening of compounds from these sources thus provides great potential for the therapy of aging and aging-related conditions.

### SUMMARY

Disclosed herein, in some embodiments, are methods of altering senescence-associated secretory phenotype (SASP) of a senescent cell in a subject in need thereof, where the method comprises administering to the subject a composition comprising alpha-ketoglutarate (AKG). In some embodiments, altering SASP comprises altering secretion of at least one, at least two, at least three, at least four, or at least five SASP factors. In some embodiments, altering SASP comprises reducing secretion of at least one, at least two, at least three, at least four, or at least five SASP factors. In some embodiments, altering SASP comprises delaying secretion of at least one, at least two, at least three, at least four, or at least five SASP factors. In some embodiments, altering SASP does not include killing or inducing killing of senescent cells. In some embodiments, altering SASP does not include inducing apoptosis of senescent cells.

Disclosed herein, in some embodiments, are methods of treating an age-related condition or age-related phenotype in a subject comprising administering to the subject a senescence-associated secretory phenotype (SASP)-altering agent in an amount that does not induce killing of senescent cells. In some embodiments, the amount does not induce apoptosis of senescent cells. In some embodiments, the SASP-altering agent alters secretion of at least one, at least two, at least three, at least four, or at least five SASP factors. In some embodiments, altering secretion comprises reducing secretion of at least one, at least two, at least three, at least four, or at least five SASP factors. In some embodiments, altering secretion comprises delaying secretion of at least one, at least two, at least three, at least four, or at least five SASP factors.

In some embodiments, the SASP factors comprise an interleukin (IL), a chemokine, an inflammatory factor, a growth factor, a protease, an extracellular matrix component, or a combination thereof.

In some embodiments, the composition further delays or reverses an age-related condition or an age-related phenotype. In some embodiments, the age-related condition or phenotype comprises osteoporosis, obesity, type-2 diabetes, macular degeneration, or an autoimmune disease. In some embodiments, the age-related condition or phenotype comprises sarcopenia. In some embodiments, the age-related condition or phenotype comprises impaired cognition. In some embodiments, the age-related condition or phenotype comprises decreased stem cell production. In some embodiments, the composition further enhances adult stem cell function. In some embodiments, the age-related condition or phenotype comprises increased inflammation. In some embodiments, the age-related condition or phenotype comprises age-related epigenetic changes. In some embodiments, epigenetic changes comprises changes in DNA methylation profile. In some embodiments, the composition induces or promotes collagen synthesis. In some embodiments, the composition treats, delays onset, or delays progression of frailty. In some embodiments, the composition extends healthspan, and/or compresses morbidity, and/or helps to maintain health. In some embodiments, the composition maintains hair density, maintains hair pigmentation, and/or induces or promotes the re-growing of hair. In some embodiments, the composition alters SASP. In some embodiments, the composition improves gait and/or balance, and/or locomotion. In some embodiments, the composition improves exercise endurance, and/or increases exercise efficiency and metabolism.

In some embodiments, prior to administering the SASP-altering agent to the subject, a biological sample is collected from the subject. In some embodiments, a senescence stimulus is applied to the control biological sample and the test biological sample before the SASP-altering agent is applied to the test biological sample, and the number of senescent cells is measured in the control biological sample and the test biological sample to assess killing of senescent cells. In some embodiments, at least one, at least two, at least three, at least four, or at least five senescent markers comprise p16^{INK4a}. In some embodiments, at least one, at least two, at least three, at least four, or at least five senescent markers comprise p21^{WAF1}.

In some embodiments, the subject is pre-screened for plasma AKG level. In some embodiments, the subject has reduced plasma AKG level.

In some embodiments, the subject is pre-screened for a DNA methylation profile. In some embodiments, the composition alters the DNA methylation profile of the subject. In some embodiments, the altered DNA methylation profile of the subject is comparable to the second control subject who is younger than the subject.

In some embodiments, the SASP-altering agent comprises alpha-ketoglutarate. In some embodiments, the AKG is a salt of AKG. In some embodiments, the salt is a calcium salt of AKG (Ca-AKG).

In some embodiments, the composition consists essentially of alpha-ketoglutarate (AKG). In some embodiments, the AKG in the composition consists essentially of a salt of AKG. In some embodiments, the AKG in the composition consists essentially of a a calcium salt of AKG (Ca-AKG).

In some embodiments, the composition is administered to the subject to achieve a therapeutically effective amount of Ca-AKG. In some embodiments, the composition comprises at least 250 mg of Ca-AKG. In some embodiments, the composition comprises at least 350 mg of Ca-AKG. In some embodiments, the composition comprises at least 500 mg of Ca-AKG. In some embodiments, the therapeutically effective amount of Ca-AKG is at least 1000 mg. In some embodiments, the composition comprises about 350 mg of Ca-AKG to about 750 mg Ca-AKG. In some embodiments, the composition comprises about 500 mg of Ca-AKG about 750 mg Ca-AKG. In some embodiments, the composition comprises about 500 mg of Ca-AKG about 600 mg Ca-AKG.

In some embodiments, the composition comprises a vitamin. In some embodiments, the composition further comprises vitamin A. In some embodiments, the amount of vitamin A is from 100 mcg to 3000 mcg. In some embodiments, the amount of vitamin A is from 200 mcg to 1000 mcg. In some embodiments, the amount of vitamin A is about 250 mcg. In some embodiments, the amount of vitamin A is about 450 mcg. In some embodiments, the amount of vitamin A is about 650 mcg. In some embodiments, the vitamin A is retinyl palmitate. In some embodiments, the vitamin is vitamin D. In some embodiments, the amount of vitamin D is from 50 IU to 3000 IU. In some embodiments, the amount of vitamin D is from 200 IU to 2000 IU. In some embodiments, the amount of vitamin D is about 250 IU. In some embodiments, the amount of vitamin D is about 500 IU. In some embodiments, the amount of vitamin D is about 750 IU. In some embodiments, the vitamin D is cholecalciferol. In some embodiments, the composition further comprises vitamin D3. In some embodiments, the composition comprises about 12.5 mcg (500 IU) of vitamin D3.

In some embodiments, the composition is administered twice daily. In some embodiments, the composition is administered three times daily. In some embodiments, the composition is administered once a week. In some embodiments, the composition is administered once a month. In some embodiments, the composition is administered to the subject for at least 3 months.

In some embodiments, the subject is a mammal. In some embodiments, the mammal is a human. In some embodiments, the mammal is a dog. In some embodiments, the mammal is a cat. In some embodiments, the mammal is livestock. In some embodiments, the subject is a male. In some embodiments, the subject is a female.

In some embodiments, the composition further comprises a pharmaceutically acceptable excipient selected from the group consisting of antiadherents, binders, coatings, colors, disintegrants, flavoring agents, antioxidants, sweetening agents, glidants, lubricants, preservatives, preservatives, sorbents, surfactants, vehicles. and combinations thereof.

In some embodiments, the composition further comprises a sweetener. In some embodiments, the sweetener is isomalt. In some embodiments, the composition further comprises wax. In some embodiments, the wax is carnauba wax and/or rice bran wax. In some embodiments, the composition further comprises one or more excipients. In some embodiments, the composition further comprises a first lubricant. In some embodiments, the first lubricant is stearic acid. In some embodiments, the composition comprises a second lubricant. In some embodiments, the second lubricant is magnesium stearate. In some embodiments, the composition comprises a glidant. In some embodiments, the glidant is silica. In some embodiments, the calcium alpha-ketoglutarate is calcium alpha-ketoglutarate monohydrate.

Disclosed herein, in some embodiments, are compositions comprising: 500-550 mg, or 525 mg of calcium alpha-ketoglutarate monohydrate, isomalt, vegetable wax (carnauba and/or rice bran), stearic acid, magnesium stearate, and silica.

Disclosed herein, in some embodiments, are compositions comprising: 500-550 mg of calcium alpha-ketoglutarate monohydrate; optionally 450 mcg of retinyl palmitate; and further comprising isomalt, vegetable wax (carnauba and/or rice bran), stearic acid, magnesium stearate, and silica.

Disclosed herein, in some embodiments, are compositions comprising: 500-550 mg of calcium alpha-ketoglutarate monohydrate; optionally 12.5 mcg (500 IU) of cholecalciferol; and further comprising isomalt, vegetable wax (carnauba and/or rice bran), stearic acid, magnesium stearate, and silica.

### BRIEF DESCRIPTION OF THE DRAWINGS

The novel and non-obvious features of the invention are set forth in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein).

FIG. 1A-FIG. 1C show AKG extends lifespan and decreases mortality. Post treatment survival plots graphed separately for Cohort-1 and Cohort-2 comparing control mice to those fed AKG in the diet starting at 18 months of age. Arrows indicate the start of the treatment. FIG. 1A shows survival curves for pooled female and male (Cohort-1, n=90) and (Cohort-2, n=94). FIG. 1B shows survival curves for female (Cohort-1, n=45) and (Cohort-2, n=46). FIG. 1C shows survival curves for male (Cohort-1, n=45) and (Cohort-2, n=48). Survival curves comparison were performed using Long-rank test, Bonferroni *P<0.05 and **P<0.01. Maximum lifespan extensions were calculated for cohort-1 using fisher's exact test statistics, *P<0.05 for female.

FIG. 2A-FIG. 2E show AKG preserves body weight in male animals (Cohort-2). FIG. 2A illustrates food consumption was measured for 3 consecutive days in different times during lifespan of control (n=6) and AKG fed (n=5) mice, the arrows at 19, 23 and 28 months show the age at which food consumption was measured. Data are mean±s.e.m, no significance change (t-test two tailed). FIG. 2B and FIG. 2D illustrate longitudinal male and female body weights, n =all live animals in the study at each time points, data are mean±s.e.m. Two way ANOVA tests check if independent variables including time and treatment (AKG) affect male and female body weight. The comparison shows significant effects of both treatment and time on male body weight (FIG. 2B) ***p<0.0001 and ***p<0.001 and effect of time on female body weight (FIG. 2D) ***p<0.001. FIG. 2C and FIG. 2E illustrate male and female body composition, n=all live animals in the study, data are mean±s.e.m.

FIG. 3A-FIG. 3D show AKG treatment extends healthspan and alleviates age-associated frailty. Separately graphed (FIG. 3A) male and (FIG. 3B) female total FI scores during lifespan, comparing control mice (blue) to those fed AKG in the diet (pink) starting at 18^{th} month. Each dot is the total score of one animal at specific age as indicated. Data are mean±s.e.m. of each group. N=all animals alive at each measurement time, *P<0.05, **P<0.01 (Student's t-test). (FIG. 3C) Male and (FIG. 3D) female individual frailty phenotypes comparison. Data are mean±s.e.m. of the group, n=all animals alive at each measurement time. *P<0.05, **P<0.01, P<0.001, ****P<0.0001 (Student's t-test).

FIG. 4A-FIG. 4D show AKG treatment prevents age-associated hair discoloration in female mice. (FIG. 4A, FIG. 4C) Overall assessment of changes in fur color index of mice in each treatment group after 9 months of treatment. Control (n=15) and AKG (n=18), Data are mean±s.e.m. ****P<0.0001 and **P<0.01 (t-test, two tailed). (FIG. 4B, FIG. 4D) Each dot is a single mouse and connecting lines tie the base line score for each mouse to its scoring after 9 months of treatment. Aging caused an increase in gray hair in control mice. AKG grouped mice had more gray hairs at baseline (18^{th} months of age) and AKG treatment reverses the hair discoloration in first cohort and prevent the hair discoloration in the second cohort of mice.

FIG. 5A-FIG. 5B show AKG treatment improves locomotion in aged mice (Cohort-2). (a, b) Locomotor activity and pedestrian locomotion was measured at the median life of the animals (28 months old). Female mice control (n=5) and AKG (n=6). Data are mean±s.e.m, *P value=0.014, **P value=0.00097 (t-test two tailed).

FIG. 6A-FIG. 6L show the results of various cardiac function and motor function tests. Treadmill exhaustion tests were performed to measure cardiovascular system and motor function for male (FIG. 6I, and FIG. 6J) and female (FIG. 6K, and FIG. 6L), age=29 month old. N=all animals alive at the time of study. No significant loss (t-test two tailed) of either cardiac function or motor function was observed.

FIG. 7A-FIG. 7B show compression of morbidity by AKG treatment. As the animal ages and gets closer to death (higher percentage of lifespan) it manifests several aging phenotypes and is at its highest multi-morbidity risk, FI is Fraity Index and total scores of 31-phenotypes are considered as morbidity score. Separately graphed (FIG. 7A) female and (FIG. 7B) male mice total frailty scores as their percentage of lifespan. AKG treatment postpones the occurrence of aging phenotypes during lifespan and compresses the morbidity risk into fewer days of life in both sexes. Each dot is the total score of one animal. Lines are mean±s.e.m. of the group. N=All animals alive at each time. **P<0.01 (two-way ANOVA).

FIG. 8A-FIG. 8B show AKG reduces inflammation in aged mice. FIG. 8A is a Heat map of 30 inflammatory cytokines and chemokines from plasma of young (age=18 months, n=11), aged control and AKG fed (29 months old, n=5) animals. All cytokines show a general trend of reduction in AKG treated group comparing to aged control. FIG. 8B exemplifies level of IL-3, IL-7, TNF-α and MIB-1B increases in old animals in comparison to young, AKG treated animals do not show an increase in cytokines levels when compared to young mice. Data are mean±s.e.m. of the group. *P<0.05 and **P<0.01 (Student's t-test).

FIG. 9A-FIG. 9D show AKG lowers the proinflammatory SASP without preventing the senescence growth arrest. FIG. 9A illustrates a qRT-PCR analysis of the indicated tissues of 30 months old mice. (FIG. 9B-FIG. 9D) Ionizing radiation (IR) was used to induce senescence in IMR-90 fibroblasts. Cells were concurrently treated with PBS (control) or 1 mM AKG and were either mock (0 Gy) or irradiated (10 Gy). All assays were performed 10 days post irradiation. (FIG. 9B) Cells were stained for senescence-associated B-galactosidase activity (left panel) or EdU incorporation (right panel). (FIG. 9C) IL-6 levels in conditioned media were determined by ELISA, normalized to cell number. FIG. 9D illustrates a qRT-PCR analysis showing expression of senescence-associated secretory phenotype (SASP) genes, normalized to actin. Each dot is one independent experiment. Data are mean±s.e.m, *p<0.05, **p<0.01 and ***p<0.001 (t-test two tailed).

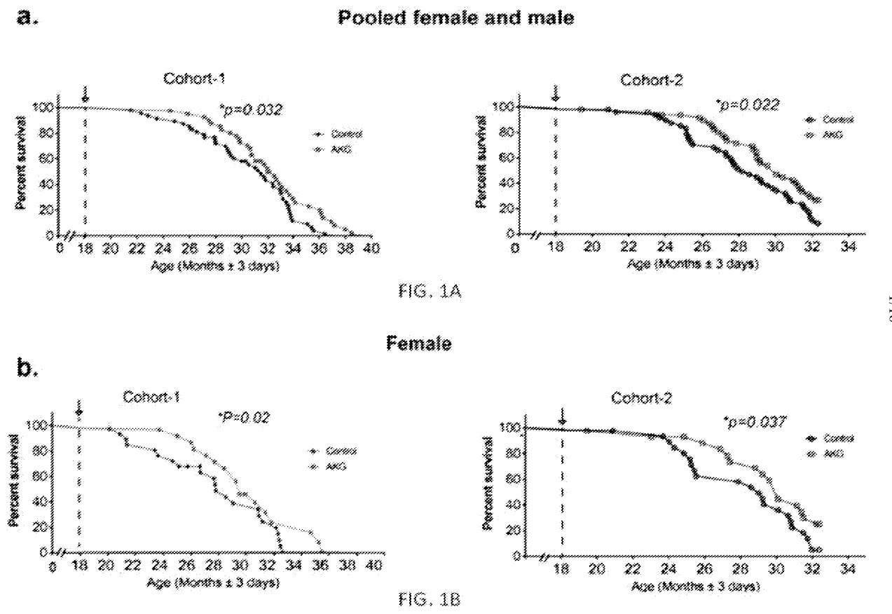

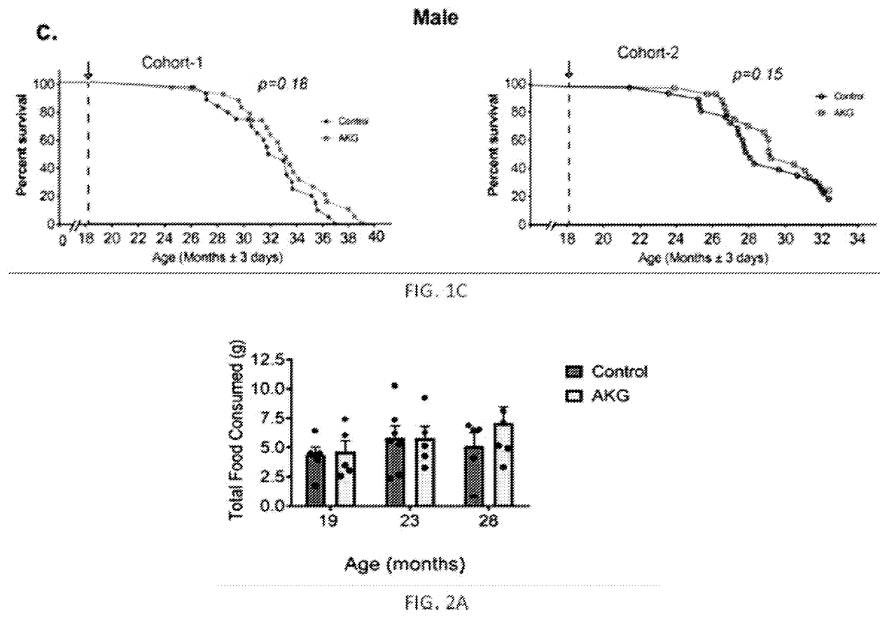

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting of the invention in any way.

### Terminology

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a disease or condition symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In certain embodiments, the term “delay” or “delaying” as related to a disease or disorder may refer to a compound that, in a statistical sample, delays or postpones the occurrence of the disorder or condition in the treated sample relative to an untreated control sample, or delays the onset or reduces the severity of one or more symptoms of the disorder or condition relative to the untreated control sample.

As used herein, “an α-ketoglutarate” or alpha-ketoglutarate or AKG, comprises α-ketoglutaric acid, salts of α-ketoglutarate, derivatives of the foregoing α-ketoglutarates (e.g., the derivatives set forth in MacKenzie, et al. (2007) Mol Cell Biol 27(9):3282-3289)), analogues of the foregoing α-ketoglutarates (e.g., phosphonate analogues (e.g., those recited in Bunik, et al. (2005) Biochemistry 44(31):10552-61), esters of α-ketoglutarate (e.g., dimethyl α-ketoglutarate and octyl α-ketoglutarate), and various species specific analogues, e.g., human α-ketoglutarate, porcine α-ketoglutarate, murine α-ketoglutarate, bovine α-ketoglutarate, and the like.

As used herein, “subject” may refer to a mammal. In some embodiments, the mammal is a human. In some embodiments, the mammal is a cat. In some embodiments, the mammal is a dog. In some embodiments, the mammal is livestock. In some embodiments, the livestock is selected from the group consisting of cattle, sheep, goats, swine, poultry, and equine animals. In some embodiments, the subject is a male. In some embodiments, the subject is a female. In some embodiments, the human is at least 18 years, at least 20 years, at least 25 years, at least 30 years, at least 35 years, at least 40 years, at least 45 years, at least 50 years, at least 55 years, at least 60 years, at least 65 years, at least 70 years, at least 75 years, or at least 80 years of age.

### Compositions

Described herein are methods and compositions for treatment of lifespan, healthspan, and aging-related disease. Further disclosed herein, in some aspects, are methods and compositions for delaying onset, or delaying progression of a disorder, reversing an age-related phenotype, extending healthspan, compressing morbidity, and altering senescence-associated secretory phenotype (SASP) of senescent cells. In some embodiments, are methods and compositions for treating frailty, for maintaining health, and for re-growing hair. In some embodiments, are methods and compositions for improving gait and balance. In some embodiments, are methods and compositions for improving locomotion. In some embodiments, are methods and compositions for improving exercise endurance. In some embodiments, are methods and compositions for improving exercise efficiency and metabolism.

In certain aspects, the disclosure provides compositions that comprise two or more compounds (individually referred to as the “active agent”) that are available for human consumption without FDA approval or generally recognized as safe (GRAS). In some embodiments, a composition comprises two or more active agent. In some embodiments, a composition comprises three or more active agents. In some embodiments, a composition comprises four or more active agents.

### Active Agents

### Alpha-Ketoglutarate

In some embodiments, the compositions disclosed herein comprise AKG. Alpha-ketoglutarate or α-ketoglutarate (Formula 1) is also known as 2-oxopentanedioic acid, 2-ketoglutaric acid, 2-oxoglutaric acid, and oxoglutaric acid. At physiological pH, α-ketoglutarate exists in one or more deprotonated forms, such as those depicted as Formulae 2. A-ketoglutarate is an intermediate in the Krebs cycle of eukaryotic organisms and is biosynthesized from isocitrate (in the Krebs cycle process) or L-glutamate (via alanine transaminase) in such organisms. Both α-ketoglutarate and its corresponding salts are commercially available, either via preparation from fermentation cultures (for example see U.S. Pat. No. 2,776,926) or chemical synthesis from closely related compounds.

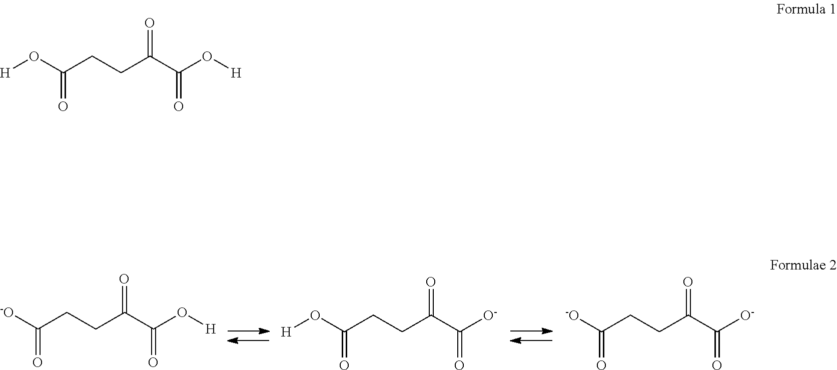

Consistent with its role in energy generation via the Krebs cycle, α-ketoglutarate is an important regulator of bioenergetics in cells and is implicated as an inhibitor of ATP synthase subunit β and an indirect inhibitor of the kinase mTOR, a consequence of partial inhibition of the mitochondrial electron transport chain.

In some embodiments, α-ketoglutarate is provided as the free acid (α-ketoglutaric acid). In some embodiments, α-ketoglutarate is provided as a mono salt or bis salt. In other embodiments, α-ketoglutarate is provided as a monosodium salt, a disodium salt, a monopotassium salt, or a dipotassium salt. In yet further embodiments, α-ketoglutarate is provided as a mono- or di-valent salt with other cations described in the U.S. FDA Orange Book. Such cations include calcium, diolamine, lithium, lysine, magnesium, meglumine, olamine, tromethamine, and zinc. In further embodiments, salts of α-ketoglutarate are provided as anhydrous salts, hemihydrates, monohydrates, or dihydrates.

Further disclosed herein, in certain aspects, are compositions that comprise α-ketoglutarate salt. In some embodiments, α-ketoglutarate is provided as a calcium salt (Ca-AKG). In some embodiments, calcium α-ketoglutarate can be a hydrate calcium α-ketoglutarate. In some embodiments, calcium α-ketoglutarate can be a mono-hydrate calcium α-ketoglutarate. In some embodiments, calcium α-ketoglutarate can be hemi-hydrate calcium α-ketoglutarate. In some embodiments, calcium α-ketoglutarate can be anhydrous calcium α-ketoglutarate.

In some embodiments, the compositions disclosed herein comprise an ester of α-ketoglutarate. In some embodiments, the ester of α-ketoglutarate is a methyl ester of α-ketoglutarate. In some embodiments, the ester of α-ketoglutarate is a dimethyl ester of α-ketoglutarate. In some embodiments, the ester of α-ketoglutarate is an ethyl ester of α-ketoglutarate. In some embodiments, the ester of α-ketoglutarate is a diethyl ester of α-ketoglutarate.

In some embodiments, AKG or Ca-AKG is combined with fish oil. In some embodiments, AKG or Ca-AKG is formulated with essential amino acids, including one or more of L-histidine, L-isoleucine, L-leucine, L-lysine, L-methionine, L-phenylalanine, L-threonine, or L-valine. In some embodiments, CaAKG or AKG is combined with nordihydroguaiaretic acid.

### Vitamin A

In some embodiments, the compositions disclosed herein comprise vitamin A. In some embodiments, the compositions disclosed herein comprise vitamin A in combination with one or more of the active agents disclosed herein (e.g. AKG or Ca-AKG, vitamin D). As disclosed herein, vitamin A can also be provided as retinoic acid (RA), retinyl palmitate (RP), or retinyl acetate.

Vitamin A is an essential organic nutrient in animals, which is converted between a variety of chemical forms in cells. These forms include retinol esters (Formula 3), retinol (Formula 4), retinal (Formula 5), and retinoic acid (Formula 6). As retinal, vitamin A is essential for the formation of rhodopsin in the eye. As retinoic acid, vitamin A plays an important role as a hormone-like growth factor for epithelial and immune cells. Relevant to age-related indications, current studies indicate that vitamin A in the form of retinoic acid activates AMPK and stimulates glucose uptake in cells (see for example Yun et al. J Biol Chem 283:33969-33974 (2008)), thus acting as an anti-diabetic agent.

Vitamin A is also present in plants in a variety of pro-vitamin A forms that are converted into retinol in animals. Such forms include alpha-carotene (Formula 7), beta-carotene (Formula 8), and gamma-carotene (Formula 9). Because of the diverse forms vitamin A, nutritional amounts of vitamin A are defined in International Units (IU), which is agnostic to the chemical form of vitamin A. For example, 1 IU is the biological equivalent of 0.3 mg retinol but 0.6 mg beta-carotene. Information regarding the IU equivalency of different forms of vitamin A is known in the art.

In some embodiments, vitamin A is provided as a retinol ester, retinol, retinal, retinoic acid, or retinoic acid salts with pharmaceutically acceptable cations as described previously. In other embodiments, vitamin A is provided as the alpha-, beta-, or gamma-carotene forms represented in formulas 7-9.

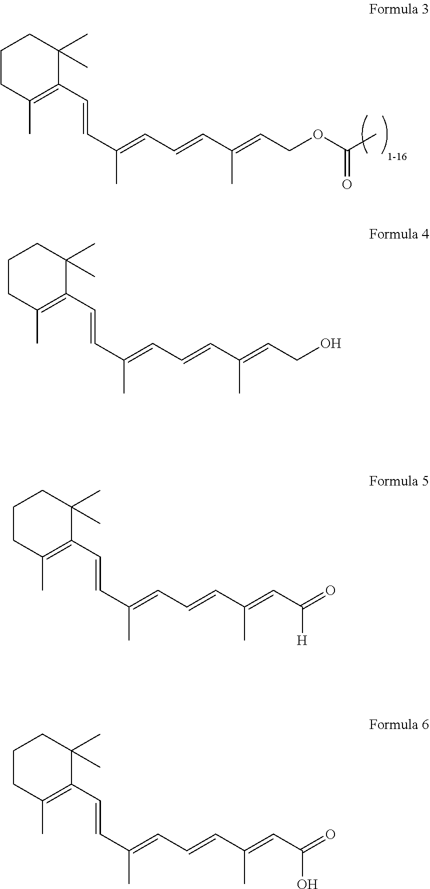

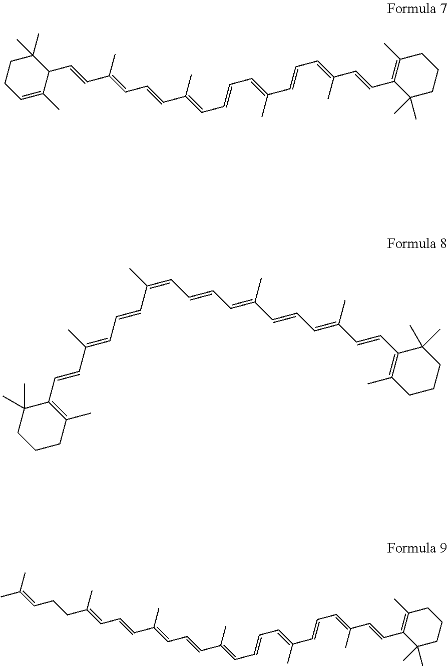

### Vitamin D

In some embodiments, the compositions disclosed herein comprise Vitamin D. In some embodiments, the compositions disclosed herein comprise vitamin D in combination with one or more of the active agents disclosed herein (AKG or Ca-AKG, vitamin A).

Vitamin D can be converted into a variety of forms. In some embodiments, vitamin D is vitamin D₂ or ergocalciferol. In some embodiments, vitamin D is vitamin D₃ or cholecalciferol. In some embodiments, vitamin D is vitamin D₄ or 22-dihydroergocalciferol. In some embodiments, vitamin D is vitamin D₅ or sitocalciferol. In some embodiments, vitamin D is vitamin D₁ or a 1:1 mixture of ergocalciferol with lumisterol.

### Vitamin E

In some embodiments, the compositions disclosed herein comprise Vitamin E. In some embodiments, the compositions disclosed herein comprise vitamin E in combination with one or more of the active agents disclosed herein (AKG or Ca-AKG, vitamin A).

Vitamin E can be converted into a variety of forms. In some embodiments, vitamin E is a tocopherol. In some embodiments, vitamin E is alpha-tocopherol. In some embodiments, vitamin E is beta-tocopherol. In some embodiments, vitamin E is gamma-tocopherol. In some embodiments, vitamin E is delta-tocopherol. In some embodiments, vitamin E is tocotrienol. In some embodiments, vitamin E is alpha-tocotrienol. In some embodiments, vitamin E is beta-tocotrienol. In some embodiments, vitamin E is gamma-tocotrienol. In some embodiments, vitamin E is delta-tocotrienol.

### Nicotinamide Adenine Dinucleotide (NAD)+ Biosynthesis Precursors

In some embodiments, the compositions disclosed herein comprise NAD+ or NADH. In some embodiments, the composition comprises a biosynthetic precursor to NAD+. In some embodiments, the biosynthetic precursor, includes, but is not limited to niacin, nicotinamide, nicotinamide riboside (NR), or nicotinamide mononucleotide (NMN). In some embodiments, the composition comprises niacin. In some embodiments, the composition comprises nicotinamide. In some embodiments, the composition comprises NR. In some embodiments, the composition comprises NMN. In some embodiments, the biosynthetic precursor to NAD+ is not NMN. In some embodiments, the composition does not comprise NMN.

In some embodiments, the biosynthetic precursor to NAD+ is combined with AKG. In some embodiments, the composition comprises AKG and niacin. In some embodiments, the composition comprises AKG and nicotinamide. In some embodiments, the composition comprises AKG and NR. In some embodiments, the composition comprises AKG and NMN.

In some embodiments, the biosynthetic precursor to NAD+ is combined with CaAKG. In some embodiments, the composition comprises CaAKG and niacin. In some embodiments, the composition comprises CaAKG and nicotinamide. In some embodiments, the composition comprises CaAKG and NR. In some embodiments, the composition comprises CaAKG and NMN.

In some embodiments, the composition comprises NAD+ or NADH with AKG. In some embodiments, the composition comprises NAD+ or NADH with CaAKG.

In some embodiments, the biosynthetic precursor to NAD+ is combined with AKG and pterostilbene. In some embodiments, the composition comprises AKG, niacin, and pterostilbene. In some embodiments, the composition comprises AKG, nicotinamide, and pterostilbene. In some embodiments, the composition comprises AKG, NR, and pterostilbene. In some embodiments, the composition comprises AKG, NMN, and pterostilbene.

In some embodiments, the biosynthetic precursor to NAD+ is combined with CaAKG and pterostilbene. In some embodiments, the composition comprises CaAKG, niacin, and pterostilbene. In some embodiments, the composition comprises CaAKG, nicotinamide, and pterostilbene. In some embodiments, the composition comprises CaAKG, NR, and pterostilbene. In some embodiments, the composition comprises CaAKG, NMN, and pterostilbene.

In some embodiments, the composition comprises NAD+ or NADH with AKG and pterostilbene. In some embodiments, the composition comprises NAD+ or NADH with CaAKG and pterostilbene.

### Mitochondrial Targeting Compounds

In some embodiments, the compositions disclosed herein comprise a compound which targets mitochondria. In some embodiments, the compound which targets mitochondria includes, but is not limited to alpha-lipoic acid, L-carnitine, coenzyme Q10 (CoQ10 (ubiquinone), vitamin E, vitamin C (ascorbic acid), pantothenic acid (Vitamin B5), or astaxanthin. In some embodiments, the vitamin E is alpha-tocopherol acetate.

In some embodiments, the compound which targets mitochondria is alpha-lipoic acid. In some embodiments, the compound which targets mitochondria is L-carnitine. In some embodiments, the compound which targets mitochondria is coenzyme Q10 (CoQ10 (ubiquinone). In some embodiments, the compound which targets mitochondria is vitamin E. In some embodiments, the compound which targets mitochondria is vitamin C. In some embodiments, the compound which targets mitochondria is pantothenic acid. In some embodiments, the compound which targets mitochondria is astaxanthin.

In some embodiments, the compound which targets mitochondria is combined with AKG. In some embodiments, the composition comprises AKG and alpha-lipoic acid. In some embodiments, the composition comprises AKG and L-carnitine. In some embodiments, the composition comprises AKG and coenzyme Q10. In some embodiments, the composition comprises AKG and vitamin E. In some embodiments, the composition comprises AKG and vitamin C. In some embodiments, the composition comprises AKG and pantothenic acid. In some embodiments, the composition comprises AKG and astaxanthin.

In some embodiments, the compound which targets mitochondria is combined with CaAKG. In some embodiments, the composition comprises CaAKG and alpha-lipoic acid. In some embodiments, the composition comprises CaAKG and L-carnitine. In some embodiments, the composition comprises CaAKG and coenzyme Q10. In some embodiments, the composition comprises CaAKG and vitamin E. In some embodiments, the composition comprises CaAKG and vitamin C. In some embodiments, the composition comprises CaAKG and pantothenic acid. In some embodiments, the composition comprises CaAKG and astaxanthin.

### Branched-Chain Amino Acids

In some embodiments, the compositions disclosed herein comprise a branched-chain amino acid. In some embodiments, the branched-chain amino acid is, but is not limited to L-leucine, L-isoleucine, or L-valine. In some embodiments, the branched-chain amino acid is L-leucine. In some embodiments, the branched-chain amino acid is L-isoleucine. In some embodiments, the branched-chain amino acid is L-valine.

In some embodiments, the branched-chain amino acid is combined with AKG. In some embodiments, the composition comprises AKG and L-leucine. In some embodiments, the composition comprises AKG and L-isoleucine. In some embodiments, the composition comprises AKG and L-valine.

In some embodiments, the branched-chain amino acid is combined with CaAKG. In some embodiments, the composition comprises CaAKG and L-leucine. In some embodiments, the composition comprises CaAKG and L-isoleucine. In some embodiments, the composition comprises CaAKG and L-valine.

### Alpha-Keto Acids

In some embodiments, the compositions disclosed herein comprise an alpha-keto acid. In some embodiments, the alpha-keto acid includes, but is not limited to, 3-methyl-2-oxobutyrate, 3-methyl-2-oxovalerate, or 4-methyl-2-oxovalerate (α-ketoisocaproic acid). In some embodiments, the alpha-keto acid is 3-methyl-2-oxobutyrate. In some embodiments, the alpha-keto acid is 3-methyl-2-oxovalerate. In some embodiments, the alpha-keto acid is 4-methyl-2-oxovalerate. It is to be understood that, as described herein, alpha-keto acids are analogs of AKG.

In some embodiments, the alpha-keto acid is combined with AKG. In some embodiments, the composition comprises AKG and 3-methyl-2-oxobutyrate. In some embodiments, the composition comprises AKG and 3-methyl-2-oxovalerate. In some embodiments, the composition comprises AKG and 4-methyl-2-oxovalerate (α-ketoisocaproic acid). In some embodiments, the alpha-keto acid is combined with CaAKG. In some embodiments, the composition comprises CaAKG and 3-methyl-2-oxobutyrate. In some embodiments, the composition comprises CaAKG and 3-methyl-2-oxovalerate. In some embodiments, the composition comprises CaAKG and 4-methyl-2-oxovalerate (α-ketoisocaproic acid).

### Beta-Hydroxy Acid

In some embodiments, the compositions disclosed herein comprise a beta-hydroxy acid. In some embodiments, the beta-hydroxy acid is beta-hydroxy-beta-methylbutyrate. In some embodiments, the beta-hydroxy acid is combined with AKG. In some embodiments, the composition comprises AKG and beta-hydroxy-beta-methylbutyrate. In some embodiments, the beta-hydroxy acid is combined with CaAKG. In some embodiments, the composition comprises CaAKG and beta-hydroxy-beta-methylbutyrate.

### Tyrosine Kinase Inhibitors

In some embodiments, the compositions disclosed herein comprise a tyrosine kinase inhibitor. In some embodiments, the tyrosine kinase inhibitor includes, but is not limited to, piceatannol, erlotinib, gefitinib, sorafenib, sunitinib, genistein, curcumin, carnosol, ursolic acid, daidzein, luteolin, quercetin, or senolytic agent. In some embodiments, the tyrosine kinase inhibitor is piceatannol. In some embodiments, the tyrosine kinase inhibitor is erlotinib. In some embodiments, the tyrosine kinase inhibitor is gefitinib. In some embodiments, the tyrosine kinase inhibitor is sorafenib. In some embodiments, the tyrosine kinase inhibitor is sunitinib. In some embodiments, the tyrosine kinase inhibitor is genistein. In some embodiments, the tyrosine kinase inhibitor is curcumin. In some embodiments, the tyrosine kinase inhibitor is carnosol. In some embodiments, the tyrosine kinase inhibitor is ursolic acid. In some embodiments, the tyrosine kinase inhibitor is daidzein. In some embodiments, the tyrosine kinase inhibitor is luteolin. In some embodiments, the tyrosine kinase inhibitor is quercetin. In some embodiments, the tyrosine kinase inhibitor is senolytic agent.

In some embodiments, the tyrosine kinase inhibitor is combined with AKG. In some embodiments, the composition comprises AKG and piceatannol. In some embodiments, the composition comprises AKG and erlotinib. In some embodiments, the composition comprises AKG and gefitinib. In some embodiments, the composition comprises AKG and sorafenib. In some embodiments, the composition comprises AKG and sunitinib. In some embodiments, the composition comprises AKG and genistein. In some embodiments, the composition comprises AKG and curcumin. In some embodiments, the composition comprises AKG and carnosol. In some embodiments, the composition comprises AKG and ursolic acid. In some embodiments, the composition comprises AKG and daidzein. In some embodiments, the composition comprises AKG and luteolin. In some embodiments, the composition comprises AKG and quercetin. In some embodiments, the composition comprises AKG and senolytic agent.

In some embodiments, the tyrosine kinase inhibitor is combined with CaAKG. In some embodiments, the composition comprises CaAKG and piceatannol. In some embodiments, the composition comprises CaAKG and erlotinib. In some embodiments, the composition comprises CaAKG and gefitinib. In some embodiments, the composition comprises CaAKG and sorafenib. In some embodiments, the composition comprises CaAKG and sunitinib. In some embodiments, the composition comprises CaAKG and genistein. In some embodiments, the composition comprises CaAKG and curcumin. In some embodiments, the composition comprises CaAKG and carnosol. In some embodiments, the composition comprises CaAKG and ursolic acid. In some embodiments, the composition comprises CaAKG and daidzein. In some embodiments, the composition comprises CaAKG and luteolin. In some embodiments, the composition comprises CaAKG and quercetin. In some embodiments, the composition comprises CaAKG and senolytic agent.

In some embodiments, the compositions disclosed herein comprise a senolytic agent. A senolytic agent is an agent which selectively induces apoptosis in senescent cells but not in non-senescent cells. In some embodiments, the senolytic agent is quercetin. In some embodiments, the senolytic agent is dasatinib. In some embodiments, the senolytic agent is artemisinin. In some embodiments, the senolytic agent is fisetin.

In some embodiments, the senolytic agent is combined with AKG. In some embodiments, the senolytic agent is combined with CaAKG. In some embodiments, the composition comprises quercetin and AKG. In some embodiments, the composition comprises quercetin and CaAKG. In some embodiments, the composition comprises dasatinib and AKG. In some embodiments, the composition comprises dasatinib and CaAKG. In some embodiments, the composition comprises dasatinib and quercetin. In some embodiments, the composition comprises dasatinib, quercetin, and AKG. In some embodiments, the composition comprises dasatinib, quercetin, and CaAKG. In some embodiments, the composition comprises artemisinin and AKG. In some embodiments, the composition comprises artemisinin and CaAKG. In some embodiments, the composition comprises fisetin and AKG. In some embodiments, the composition comprises fisetin and CaAKG.

### Other Active agents

In some embodiments, the compositions disclosed herein comprise D-beta-hydroxybutyrate. In some embodiments, the composition comprises D-beta-hydroxybutyrate and AKG. In some embodiments, the composition comprises D-beta-hydroxybutyrate and CaAKG.

In some embodiments, the compositions disclosed herein comprise alpha-ketobutyrate. In some embodiments, the composition comprises alpha-ketobutyrate and AKG. In some embodiments, the composition comprises alpha-ketobutyrate and CaAKG. In some embodiments, the composition comprises D-beta-hydroxybutyrate, alpha-ketobutyrate, and AKG. In some embodiments, the composition comprises D-beta-hydroxybutyrate, alpha-ketobutyrate, and CaAKG.

In some embodiments, the composition comprises Ergothioneine. In some embodiments, the composition comprises Ergothioneine, and AKG. In some embodiments, the composition comprises Ergothioneine, and CaAKG.

### Methods of Use

### Frailty

In an aspect, the disclosure provides methods for treating, delaying onset, or delaying progression of frailty using the active agents or compositions thereof described herein. The term “frailty” refers to a biological syndrome of decreased reserve and resistance to stressors due to decline in multiple physiological systems. Subjects suffering from frailty have improved likelihood of adverse health outcomes to events that stress one or more of their physiological systems. In humans, frailty frequently presents via non-specific symptoms, falls, delirium, fluctuating disability, or a combination thereof. Non-specific symptoms include extreme fatigue, unexplained weight loss, and frequent infections. Falls include hot falls (minor illness reducing postural balance below a threshold to maintain stability) or spontaneous falls (vital postural systems declining as a result of declines in vision, balance, and strength). Delirium refers to rapid onset of fluctuating confusion and impaired awareness. Fluctuating disability refers to day-to-day instability in the ability of a patient to function independently.

Various clinical scoring and evaluation systems for frailty are known to those skilled in the art and are suitable for assessing the effects of treatment on frailty. In some embodiments, frailty is evaluated in humans using the 70-item CSHA Frailty Index (see, for e.g. Theou et al. Age Ageing 42: 614-619 (2013)). A brief description of how the index is employed follows:

Items including the presence and/or severity of current diseases, ability in daily living and physical signs from the clinical and neurological examinations (see items in Table 1 below) are evaluated. Each deficit is dichotomized or trichotomized and mapped to the interval 0-1 (i.e. individual items had scores of 0, 0.33, 0.50, 0.67 or 1.0), representing the occurrence and severity of the problem. For each person, a 70-dimentional vector is constructed, such that a person with 5 deficits would have a score of 5/70=0.071.

TABLE 1
List of measures used to construct the Frailty Index

| | | |
|---|---|---|
| Self-rated health | Long-term illness | Limitations with Activities |
| In hospital last 12 months | Heart attack | High blood pressure |
| High blood cholesterol | Stroke or cerebral vascular | Diabetes or high blood sugar |
| | Disease | |
| Chronic lung disease | Asthma | Arthritis |
| Osteoporosis | Cancer | Stomach or duodenal ulcer |
| Parkinson disease | Cataracts | Hip or femoral fracture |
| Pain in any joint | Heart trouble or angina | Breathlessness |
| Persistent cough | Swollen legs | Sleeping problems |
| Falling down | Fear of falling down | Dizziness |
| Stomach or intestine | Incontinence | Dentures |
| Problems | | |
| Biting on hard foods | Eyesight | Hearing |
| Walking 100 meters | Sitting for about two hours | Getting up from a chair after |
| | | prolonged sitting |
| Climbing several | Stooping, kneeling, or | Reaching or extending arm |
| flights of Stairs | Crouching | above shoulder level |
| Pulling or pushing large | Lifting or carrying weights | Picking up a small coin from |
| Objects | over 10 pounds/5 kilos | a table |
| Dressing | Walking across a room | Bathing or showering |
| Eating | Getting in or out of bed | Using the toilet |
| Using a map to figure out | Preparing a hot meal | Shopping for groceries |
| how to get around | | |
| Making telephone calls | Taking medications | Doing work around the |
| | | house or garden |
| Managing money | Vigorous activities | Moderate Activities |
| Orientation | Mathematical performance | Verbal fluency score |
| Delayed recall test | Depression | Pessimism |
| Suicidality | Sleep | Interest |
| Appetite | Fatigue | Concentration |

Further detail on application of the CSHA frailty index (e.g. calculation of scores for individual measures) can be found in other publications in the field, for e.g. in Searle et al. A standard procedure for creating a frailty index. BMC Geriatrics 8:24 (2008). An example of the use of the CSHA frailty index in humans can be found in Example 4, where selection of pre-frail individuals for treatment and evaluation of pharmacological treatment of frailty is described.

In other embodiments, frailty is evaluated in non-human animals, such as mice. Recognized signs of frailty in mice correspond to many of those in humans, and involve metabolic (e.g. body temperature, body weight), integumental (e.g. alopecia, loss of fur color, dermatitis, loss of whiskers, grooming), physical/musculoskeletal (e.g. tumors, distended abdomen, kyphosis, tail stiffening, gait disorder, tremor, decreased forelimb grip strength, body condition/muscle wasting/obesity), vestibulocochlear/auditory (e.g. vestibular disturbance, hearing loss), ocular/nasal (e.g. cataracts, corneal opacity, eye discharge, microphthalmia, vision loss, increased menace reflex, nasal discharge), digestive/urogenital (e.g. malocclusions, rectal prolapse, vaginal/uterine/penile prolapse, diarrhea), respiratory (e.g. abnormal breathing rate or depth), and discomfort symptoms (e.g. increased mouse grimace scale, piloerection).

In some embodiments, frailty is assessed in mice via a 31-item clinical frailty index encompassing the 31 example phenotypes recited above as described in, for e.g. Whitehead et al. J Gerontol A Biol Sci Med Sci 69:621-632 (2014). Clinical examinations are performed at approximately the same time every 2 to 3 months, and involve body weight and surface temperature measurement by abdominal infrared, followed by a clinical exam to evaluate the 31 frailty phenotypes. The severity of each deficit is rated on a scale, with 0 given for no sign of a deficit, 0.5 for a mild deficit, and 1 for a severe deficit. Deficits in body weight (g) and body surface temperature (° C.) are scored in quantiles between 0 and 1 based on number of standard deviations from reference values in young adult animals (0.25, 0.5, 0.75, and 1.0) according to how many standard deviations the score varies from the mean (1 is >3 SD). The sum of the scores for each parameter produce the final 31-item frailty index, which can be compared between individual mice according to standard statistical techniques to assess frailty.

In some embodiments, frailty is assessed in mice via an abbreviated with an eight-item functional frailty index as described in, for e.g. Whitehead et al. J Gerontol A Biol Sci Med Sci 69:621-632 (2014) and Parks et al. J Gerontol A Biol Sci Med Sci. 67:217-227 (2012). In this method, 7 performance parameters based on open-field behavior of mouse subjects are assessed: 1) total distance moved in 10 minutes; 2) maximal distance moved between bouts of inactivity; 3) total duration of movement (seconds); 4) percent of total time spent moving; 5) the change in direction per unit distance moved, called meander (degrees/cm; from 0° to 180°); 6) the average velocity of movement over 10 minutes (cm/s); and 7) rearing frequency (number of occurrences/10 min). An eighth non-movement parameter, weight, is additionally assessed. Open-field assessments are performed between 10 am and noon each day. Mice are weighed and activity was recorded with automated video tracking software for 10 minutes in an open-field arena. Videos are digitized with an analog-to-digital converter and analyzed with video tracking analysis software to obtain values for the parameters used to create the eight-item frailty index. Mean and standard deviation for each of these parameters are calculated and assigned to a score quantile between 0 and 1 (0.25, 0.5, 0.75, and 1.0) according to how many standard deviations the score varies from the mean (1 is >3 SD). The parameters are added, and divided by eight to receive a frailty index score between 0 and 1 for the mouse subjects. Higher scores correspond to increasingly frail mice.

In some embodiments, the disclosure provides methods for treating, delaying onset, or delaying progression of frailty in a subject in need thereof using the compositions disclosed herein. In some aspects, the composition for treating, delaying onset, or delaying progression of frailty comprises two or more active agents.

In some embodiments, the composition for treating, delaying onset, or delaying progression of frailty in a subject in need thereof comprises alpha-ketoglutarate (AKG). In some embodiments, the composition for treating, delaying onset, or delaying progression of frailty in a subject in need thereof consists essentially of alpha-ketoglutarate (AKG).

In some embodiments, the composition for treating, delaying onset, or delaying progression of frailty in a subject in need thereof comprises the biosynthetic precursor to NAD+ and AKG. In some embodiments, the composition for treating, delaying onset, or delaying progression of frailty in a subject in need thereof comprises the biosynthetic precursor to NAD+ and Ca-AKG.

In some embodiments, the composition for treating, delaying onset, or delaying progression of frailty in a subject in need thereof comprises the biosynthetic precursor to NAD+, AKG and pterostilbene.

In some embodiments, the composition for treating, delaying onset, or delaying progression of frailty in a subject in need thereof comprises α-ketoglutarate salt. In some embodiments, the composition for treating, delaying onset, or delaying progression of frailty in a subject in need thereof consists essentially of α-ketoglutarate salt.

In some embodiments, the composition for treating, delaying onset, or delaying progression of frailty in a subject in need thereof comprises an ester of α-ketoglutarate.

In some embodiments, the composition for treating, delaying onset, or delaying progression of frailty in a subject in need thereof comprises vitamin A. In some embodiments, the composition comprises vitamin A and Ca-AKG. In some embodiments, the composition for treating, delaying onset, or delaying progression of frailty in a subject in need thereof comprises Vitamin D. In some embodiments, the composition comprises vitamin D and Ca-AKG. In some embodiments, the composition for treating, delaying onset, or delaying progression of frailty in a subject in need thereof comprises Vitamin E. In some embodiments, the composition comprises vitamin E and Ca-AKG.

In some embodiments, the composition for treating, delaying onset, or delaying progression of frailty in a subject in need thereof comprises a compound which targets mitochondria.

In some embodiments, the composition for treating, delaying onset, or delaying progression of frailty in a subject in need thereof comprises a branched-chain amino acid.

In some embodiments, the composition for treating, delaying onset, or delaying progression of frailty in a subject in need thereof comprises an alpha-keto acid. In some embodiments, the composition for treating, delaying onset, or delaying progression of frailty in a subject in need thereof comprises a beta-hydroxy acid.

In some embodiments, the composition for treating, delaying onset, or delaying progression of frailty in a subject in need thereof comprises a tyrosine kinase inhibitor.

In some embodiments, the composition for treating, delaying onset, or delaying progression of frailty in a subject in need thereof comprises a senolytic agent. In some embodiments, the senolytic agent is combined with AKG. In some embodiments, the senolytic agent is combined with CaAKG.

### Healthspan

In an aspect, the disclosure provides methods of treatment for extending healthspan using the active agents or compositions thereof disclosed herein. Healthspan refers to the period of time during which an individual meets one or more selected measures of health. An increase in healthspan refers to an extension in the period of health, according to such measures, as compared to the period of health in a control population. Examples of selected measures of health that are evaluated in a human population to assess healthspan include one or more age-related phenotypes such as energetics/metabolism (e.g. elevated insulin, insulin resistance, elevated fasting blood glucose+GTT, elevated Hb A1c, adiponectin, elevated DEXA/abdominal adiposity, increased IGF-I, decreased T3, elevated low-density lipoprotein, decreased high-density lipoprotein, elevated triglycerides), skeletal muscle function (e.g. decreased hand grip strength, decreased mobility), cardiopulmonary function (e.g. decreased VO2 max, elevated blood pressure, decreased pulse wave velocity, intima media thickness, decreased left ventricular diastolic function, increased left ventricular diastolic pressure), inflammation and immune function (e.g. decreased lymphocyte number, decreased lymphoid/myeloid ratio, elevated CRP, elevated IL-6, elevated TNF-α), sensory function (e.g. decreased visual acuity, decreased nerve conduction velocity), cognition (e.g. decreased score on cognitive function tests like the MMSE/AMTS/GPAC, impaired activity via fMRIs), cellular senescence (e.g. graying hair), and pathology (e.g. renal, cardiac, pulmonary, breast, or prostate tissue evaluation to evaluate age-related tissue hypertrophy or dysplasia).

Examples of non-human animals used for assessment of lifespan-extension or healthspan interventions include C. elegans, D. melanogaster, and M. musculus. Use of D. melanogaster or M. musculus to evaluate lifespan or healthspan interventions are found in, for e.g. Bauer et al. Proc Natl Acad Sci U S A. 101:12980-5 (2004) and Selman et al. FASEB J 22:807-18 (2008).

In some embodiments, the disclosure provides methods of extending healthspan using the compositions disclosed herein. In some aspects, the composition comprises two or more active agents described herein.

In some embodiments, the composition for extending healthspan in a subject in need thereof comprises alpha-ketoglutarate (AKG). In some embodiments, the composition for extending healthspan in a subject in need thereof consists essentially of alpha-ketoglutarate (AKG).

In some embodiments, the composition for extending healthspan in a subject in need thereof comprises the biosynthetic precursor to NAD+ and AKG. In some embodiments, the composition for extending healthspan in a subject in need thereof comprises the biosynthetic precursor to NAD+ and Ca-AKG.

In some embodiments, the composition for extending healthspan in a subject in need thereof comprises the biosynthetic precursor to NAD+, AKG and pterostilbene.

In some embodiments, the composition for extending healthspan in a subject in need thereof comprises α-ketoglutarate salt. In some embodiments, the composition for extending healthspan in a subject in need thereof consists essentially of α-ketoglutarate salt.

In some embodiments, the composition for extending healthspan in a subject in need thereof comprises an ester of α-ketoglutarate.

In some embodiments, the composition for extending healthspan in a subject in need thereof comprises vitamin A. In some embodiments, the composition comprises vitamin A and Ca-AKG. In some embodiments, the composition for extending healthspan in a subject in need thereof comprises Vitamin D. In some embodiments, the composition comprises vitamin D and Ca-AKG. In some embodiments, the composition for extending healthspan in a subject in need thereof comprises Vitamin E. In some embodiments, the composition comprises vitamin E and Ca-AKG.

In some embodiments, the composition for extending healthspan in a subject in need thereof comprises a compound which targets mitochondria.

In some embodiments, the composition for extending healthspan in a subject in need thereof comprises a branched-chain amino acid.

In some embodiments, the composition for extending healthspan in a subject in need thereof comprises an alpha-keto acid. In some embodiments, the composition for extending healthspan in a subject in need thereof comprises a beta-hydroxy acid.

In some embodiments, the composition for extending healthspan in a subject in need thereof comprises a tyrosine kinase inhibitor.

In some embodiments, the composition for extending healthspan in a subject in need thereof comprises a senolytic agent. In some embodiments, the senolytic agent is combined with AKG. In some embodiments, the senolytic agent is combined with CaAKG.

### Compression of Morbidity

In an aspect, the disclosure provides methods of compressing morbidity using the active agents or compositions thereof described herein. Compression of morbidity occurs if the age of first appearance of aging manifestations and chronic disease symptoms increases more rapidly than life expectancy. The period between marker of morbidity (e.g. first heart attack, first dyspnea from emphysema, first disability from osteoarthritis, first memory loss of a certain magnitude) and the end of life is shortened when the average onset age of the marker increases more rapidly than life expectancy from the same age. This disproportionally increases the healthy years of life, and dramatically reduces the end stage costs of healthcare.

In an aspect, the disclosure provides methods of compressing morbidity using the active agents or compositions thereof described herein. In some aspects, the composition comprises two or more active agents as described herein.

In some embodiments, the composition for compressing morbidity in a subject in need thereof comprises alpha-ketoglutarate (AKG). In some embodiments, the composition for compressing morbidity in a subject in need thereof consists essentially of alpha-ketoglutarate (AKG).

In some embodiments, the composition for compressing morbidity in a subject in need thereof comprises the biosynthetic precursor to NAD+ and AKG. In some embodiments, the composition for compressing morbidity in a subject in need thereof comprises the biosynthetic precursor to NAD+ and Ca-AKG.

In some embodiments, the composition for compressing morbidity in a subject in need thereof comprises the biosynthetic precursor to NAD+, AKG and pterostilbene.

In some embodiments, the composition for compressing morbidity in a subject in need thereof comprises α-ketoglutarate salt. In some embodiments, the composition for compressing morbidity in a subject in need thereof consists essentially of α-ketoglutarate salt.

In some embodiments, the composition for compressing morbidity in a subject in need thereof comprises an ester of α-ketoglutarate.

In some embodiments, the composition for compressing morbidity in a subject in need thereof comprises vitamin A. In some embodiments, the composition comprises vitamin A and Ca-AKG. In some embodiments, the composition for compressing morbidity in a subject in need thereof comprises Vitamin D. In some embodiments, the composition comprises vitamin D and Ca-AKG. In some embodiments, the composition for compressing morbidity in a subject in need thereof comprises Vitamin E. In some embodiments, the composition comprises vitamin E and Ca-AKG.

In some embodiments, the composition for compressing morbidity in a subject in need thereof comprises a compound which targets mitochondria.

In some embodiments, the composition for compressing morbidity in a subject in need thereof comprises a branched-chain amino acid.

In some embodiments, the composition for compressing morbidity in a subject in need thereof comprises an alpha-keto acid. In some embodiments, the composition for compressing morbidity in a subject in need thereof comprises a beta-hydroxy acid.

In some embodiments, the composition for compressing morbidity in a subject in need thereof comprises a tyrosine kinase inhibitor.

In some embodiments, the composition for compressing morbidity in a subject in need thereof comprises a senolytic agent. In some embodiments, the senolytic agent is combined with AKG. In some embodiments, the senolytic agent is combined with CaAKG.

### Methods of Helping to Maintaining Health

In an aspect, the disclosure provides methods of helping to maintain health using the active agents or compositions thereof described herein. In some embodiments, helping to maintain health comprises helping to maintain a healthy metabolism. In some embodiments, helping to maintain a healthy metabolism comprises helping to maintain a healthy body temperature. In some embodiments, helping to maintain a healthy metabolism comprises helping to maintain a healthy body weight.

In some embodiments, helping to maintain health comprises helping to maintain a healthy musculoskeletal system. In some embodiments, helping to maintain a healthy musculoskeletal system comprises helping to maintain grip strength. In some embodiments, helping to maintain a healthy musculoskeletal system comprises helping to maintain normal spine curvature. In some embodiments, helping to maintain a healthy musculoskeletal system comprises helping to maintain normal gait. In some embodiments, helping to maintain a healthy musculoskeletal system comprises helping to maintain normal muscle mass.

In some embodiments, helping to maintain health comprises helping to maintain a healthy auditory system. In some embodiments, helping to maintain health comprises helping to maintain a healthy ocular system. In some embodiments, helping to maintain health comprises helping to maintain normal vision. In some embodiments, helping to maintain health comprises helping to maintain a healthy digestive system. In some embodiments, helping to maintain health comprises helping to maintain a healthy urogenital tract. In some embodiments, helping to maintain health comprises helping to maintain a healthy respiratory system.

In some embodiments, helping to maintain health comprises helping to maintain a healthy cardiovascular system. In some embodiments, helping to maintain health comprises helping to maintain a healthy body weight.

In some embodiments, helping to maintain health comprises helping to maintain a healthy senescence-associated secretory phenotype (SASP). In some embodiments, helping to maintain health comprises helping to maintain a healthy level of secretion of SASP factors. In some embodiments, helping to maintain health comprises helping to maintain a healthy level of secretion of SASP factors, wherein SASP factors comprise an interleukin (IL), a chemokine, an inflammatory factor, a growth factor, a protease, an extracellular matrix component, or a combination thereof. In some embodiments, helping to maintain health comprises helping to maintain healthy level of secretion of SASP factors comprising IL-1β, IL-3, IL-6, IL-7, MIP-1β, TNF-α, CCL2, MMP3, or a combination thereof.

In some embodiments, the disclosure provides methods of maintaining health comprise using the compositions disclosed herein. In some aspects, the composition comprises two or more active agents as described herein.

In some embodiments, the composition for helping to maintain health in a subject in need thereof comprises alpha-ketoglutarate (AKG). In some embodiments, the composition for helping to maintain health in a subject in need thereof consists essentially of alpha-ketoglutarate (AKG).

In some embodiments, the composition for helping to maintain health in a subject in need thereof comprises the biosynthetic precursor to NAD+ and AKG. In some embodiments, the composition for helping to maintain health in a subject in need thereof comprises the biosynthetic precursor to NAD+ and Ca-AKG.

In some embodiments, the composition for helping to maintain health in a subject in need thereof comprises the biosynthetic precursor to NAD+, AKG and pterostilbene.

In some embodiments, the composition for helping to maintain health in a subject in need thereof comprises α-ketoglutarate salt. In some embodiments, the composition for helping to maintain health in a subject in need thereof consists essentially of α-ketoglutarate salt.

In some embodiments, the composition for helping to maintain health in a subject in need thereof comprises an ester of α-ketoglutarate.

In some embodiments, the composition for helping to maintain health in a subject in need thereof comprises vitamin A. In some embodiments, the composition comprises vitamin A and Ca-AKG. In some embodiments, the composition for helping to maintain health in a subject in need thereof comprises Vitamin D. In some embodiments, the composition comprises vitamin D and Ca-AKG. In some embodiments, the composition for helping to maintain health in a subject in need thereof comprises Vitamin E. In some embodiments, the composition comprises vitamin E and Ca-AKG.

In some embodiments, the composition for helping to maintain health in a subject in need thereof comprises a compound which targets mitochondria.

In some embodiments, the composition for helping to maintain health in a subject in need thereof comprises a branched-chain amino acid.

In some embodiments, the composition for helping to maintain health in a subject in need thereof comprises an alpha-keto acid. In some embodiments, the composition for helping to maintain health in a subject in need thereof comprises a beta-hydroxy acid.

In some embodiments, the composition for helping to maintain health in a subject in need thereof comprises a tyrosine kinase inhibitor.

In some embodiments, the composition for helping to maintain health in a subject in need thereof comprises a senolytic agent. In some embodiments, the senolytic agent is combined with AKG. In some embodiments, the senolytic agent is combined with CaAKG.

### Maintaining Har Density

In an aspect, the disclosure provides methods of maintaining hair density using the active agents or compositions thereof described herein. In some embodiments, hair density is maintained in a subject that has alopecia. In some embodiments, maintaining hair density comprises maintaining a healthy scalp. In some embodiments, maintaining hair density comprises maintaining healthy hair follicles. In some embodiments, maintaining hair density comprises maintaining healthy hair shafts. In some embodiments, maintaining hair density comprises maintaining healthy hair bulbs.

In some embodiments, the disclosure provides methods of maintaining hair density comprise using the compositions disclosed herein. In some aspects, the composition comprises two or more active agents as described herein.

In some embodiments, the composition for maintaining hair density in a subject in need thereof comprises alpha-ketoglutarate (AKG). In some embodiments, the composition for maintaining hair density in a subject in need thereof consists essentially of alpha-ketoglutarate (AKG).

In some embodiments, the composition for maintaining hair density in a subject in need thereof comprises the biosynthetic precursor to NAD+ and AKG. In some embodiments, the composition for maintaining hair density in a subject in need thereof comprises the biosynthetic precursor to NAD+ and Ca-AKG.

In some embodiments, the composition for maintaining hair density in a subject in need thereof comprises the biosynthetic precursor to NAD+, AKG and pterostilbene.

In some embodiments, the composition for maintaining hair density in a subject in need thereof comprises α-ketoglutarate salt. In some embodiments, the composition for maintaining hair density in a subject in need thereof consists essentially of α-ketoglutarate salt.

In some embodiments, the composition for maintaining hair density in a subject in need thereof comprises an ester of α-ketoglutarate.

In some embodiments, the composition for maintaining hair density in a subject in need thereof comprises vitamin A. In some embodiments, the composition comprises vitamin A and Ca-AKG. In some embodiments, the composition for maintaining hair density in a subject in need thereof comprises Vitamin D. In some embodiments, the composition comprises vitamin D and Ca-AKG. In some embodiments, the composition for maintaining hair density in a subject in need thereof comprises Vitamin E. In some embodiments, the composition comprises vitamin E and Ca-AKG.

In some embodiments, the composition for maintaining hair density in a subject in need thereof comprises a compound which targets mitochondria.

In some embodiments, the composition for maintaining hair density in a subject in need thereof comprises a branched-chain amino acid.

In some embodiments, the composition for maintaining hair density in a subject in need thereof comprises an alpha-keto acid. In some embodiments, the composition for maintaining hair density in a subject in need thereof comprises a beta-hydroxy acid.

In some embodiments, the composition for maintaining hair density in a subject in need thereof comprises a tyrosine kinase inhibitor.

In some embodiments, the composition for maintaining hair density in a subject in need thereof comprises a senolytic agent. In some embodiments, the senolytic agent is combined with AKG. In some embodiments, the senolytic agent is combined with CaAKG.

### Maintaining Hair Pigmentation

In an aspect, the disclosure provides methods of maintaining hair pigmentation using the active agents or compositions thereof described herein. In some embodiments, maintaining hair pigmentation comprises maintaining a normal level of melanin. In some embodiments, hair pigmentation is maintained in a subject that has low vitamin B12 levels. In some embodiments, maintaining hair pigmentation comprises maintaining a normal level of melanocyte stem cells.

In some embodiments, the disclosure provides methods of maintaining hair pigmentation comprises using the compositions disclosed herein. In some aspects, the composition comprises two or more active agents as described herein.

In some embodiments, the composition for maintaining hair pigmentation in a subject in need thereof comprises alpha-ketoglutarate (AKG). In some embodiments, the composition for maintaining hair pigmentation in a subject in need thereof consists essentially of alpha-ketoglutarate (AKG).

In some embodiments, the composition for maintaining hair pigmentation in a subject in need thereof comprises the biosynthetic precursor to NAD+ and AKG. In some embodiments, the composition for maintaining hair pigmentation in a subject in need thereof comprises the biosynthetic precursor to NAD+ and Ca-AKG.

In some embodiments, the composition for maintaining hair pigmentation in a subject in need thereof comprises the biosynthetic precursor to NAD+, AKG and pterostilbene.

In some embodiments, the composition for maintaining hair pigmentation in a subject in need thereof comprises α-ketoglutarate salt. In some embodiments, the composition for maintaining hair pigmentation in a subject in need thereof consists essentially of α-ketoglutarate salt.

In some embodiments, the composition for maintaining hair pigmentation in a subject in need thereof comprises an ester of α-ketoglutarate.

In some embodiments, the composition for maintaining hair pigmentation in a subject in need thereof comprises vitamin A. In some embodiments, the composition comprises vitamin A and Ca-AKG. In some embodiments, the composition for maintaining hair pigmentation in a subject in need thereof comprises Vitamin D. In some embodiments, the composition comprises vitamin D and Ca-AKG. In some embodiments, the composition for maintaining hair pigmentation in a subject in need thereof comprises Vitamin E. In some embodiments, the composition comprises vitamin E and Ca-AKG.

In some embodiments, the composition for maintaining hair pigmentation in a subject in need thereof comprises a compound which targets mitochondria.

In some embodiments, the composition for maintaining hair pigmentation in a subject in need thereof comprises a branched-chain amino acid.

In some embodiments, the composition for maintaining hair pigmentation in a subject in need thereof comprises an alpha-keto acid. In some embodiments, the composition for maintaining hair pigmentation in a subject in need thereof comprises a beta-hydroxy acid.

In some embodiments, the composition for maintaining hair pigmentation in a subject in need thereof comprises a tyrosine kinase inhibitor.

In some embodiments, the composition for maintaining hair pigmentation in a subject in need thereof comprises a senolytic agent. In some embodiments, the senolytic agent is combined with AKG. In some embodiments, the senolytic agent is combined with CaAKG.

### Hair Re-Growth

In some embodiments, the disclosure provides methods of re-growing hair in a subject in need thereof using the active agents or compositions thereof described herein. In some embodiments, the subject has alopecia. In some embodiments, the subject has male or female pattern baldness. In some embodiments, the subject has alopecia areata. In some embodiments, the subject has telogen effluvium. In some embodiments, the subject has anagen effluvium. In some embodiments, the subject has alopecial totalis. In some embodiments, the subject has alopecia universalis. In some embodiments, the subject has alopecia barbae. In some embodiments, the subject has alopecia mucinosa. In some embodiments, the subject has traction alopecia. In some embodiments, the subject has scarring alopecia. In some embodiments, the subject has trichotillomania.

In some embodiments, the composition for re-growing hair in a subject in need thereof comprises alpha-ketoglutarate (AKG). In some embodiments, the composition for re-growing hair in a subject in need thereof consists essentially of alpha-ketoglutarate (AKG).

In some embodiments, the composition for re-growing hair in a subject in need thereof comprises the biosynthetic precursor to NAD+ and AKG. In some embodiments, the composition for re-growing hair in a subject in need thereof comprises the biosynthetic precursor to NAD+ and Ca-AKG.

In some embodiments, the composition for re-growing hair in a subject in need thereof comprises the biosynthetic precursor to NAD+, AKG and pterostilbene.

In some embodiments, the composition for re-growing hair in a subject in need thereof comprises α-ketoglutarate salt. In some embodiments, the composition for re-growing hair in a subject in need thereof consists essentially of α-ketoglutarate salt.

In some embodiments, the composition for re-growing hair in a subject in need thereof comprises an ester of α-ketoglutarate.

In some embodiments, the composition for re-growing hair in a subject in need thereof comprises vitamin A. In some embodiments, the composition comprises vitamin A and Ca-AKG. In some embodiments, the composition for re-growing hair in a subject in need thereof comprises Vitamin D. In some embodiments, the composition comprises vitamin D and Ca-AKG. In some embodiments, the composition for re-growing hair in a subject in need thereof comprises Vitamin E. In some embodiments, the composition comprises vitamin E and Ca-AKG.

In some embodiments, the composition for re-growing hair in a subject in need thereof comprises a compound which targets mitochondria.

In some embodiments, the composition for re-growing hair in a subject in need thereof comprises a branched-chain amino acid.

In some embodiments, the composition for re-growing hair in a subject in need thereof comprises an alpha-keto acid. In some embodiments, the composition for re-growing hair in a subject in need thereof comprises a beta-hydroxy acid.

In some embodiments, the composition for re-growing hair in a subject in need thereof comprises a tyrosine kinase inhibitor.

In some embodiments, the composition for re-growing hair in a subject in need thereof comprises a senolytic agent. In some embodiments, the senolytic agent is combined with AKG. In some embodiments, the senolytic agent is combined with CaAKG.

### Gait, Locomotion and Balance

In some embodiments, the disclosure provides methods for decreasing loss of a normal gait, and/or improving gait in a subject in need thereof using the active agents or compositions thereof described herein. Illustrative diseases that include or are characterized by gait and/or locomotion dysfunction include, but are not limited to, trauma, degenerative intervertebral disk diseases, such as spondylosis, spinal canal stenosis, disk hernia, and the like, osteoarthritis, such as knee OA, hip OA, and the like, ataxia, dystonia, such as cervical dystonia, chorea, functional movement disorder, Huntington's disease, multiple system atrophy, myoclonus, Parkinson's disease, progressive supranuclear palsy, restless legs syndrome, tardive dyskinesia, tremor, Wilson's disease, and the like. Illustrative diseases that include or are characterized by balance dysfunction include, but are not limited, vertigo, benign paroxysmal positional vertigo (BPPV), Meniere's disease, migraine, acoustic neuroma, vestibular neuritis, Ramsay Hunt syndrome, head injury, motion sickness, ear infections, inner ear problems, poor blood circulation, side effects from certain medications, brain chemical imbalance, low blood pressure, high blood pressure, and the like. In some embodiments, the disclosure provides methods for decreasing loss of locomotion, and/or improving locomotion in a subject in need thereof using the active agents or compositions thereof described herein. In some embodiments, the disclosure provides methods for decreasing loss of balance, and/or improving balance in a subject in need thereof using the active agents or compositions thereof described herein. In some embodiments, the subject has a movement disorder or chorea. In some embodiments, the subject has a neurological or neurodegenerative disease.

In some embodiments, the composition for decreasing loss of and/or improving gait, locomotion, and/or balance in a subject in need thereof comprises alpha-ketoglutarate (AKG). In some embodiments, the composition for decreasing loss of and/or improving gait, locomotion, and/or balance in a subject in need thereof consists essentially of alpha-ketoglutarate (AKG).

In some embodiments, the composition for decreasing loss of and/or improving gait, locomotion, and/or balance in a subject in need thereof comprises the biosynthetic precursor to NAD+ and AKG. In some embodiments, the composition for decreasing loss of and/or improving gait, locomotion, and/or balance in a subject in need thereof comprises the biosynthetic precursor to NAD+ and Ca-AKG.

In some embodiments, the composition for decreasing loss of and/or improving gait, locomotion, and/or balance in a subject in need thereof comprises the biosynthetic precursor to NAD+, AKG and pterostilbene.

In some embodiments, the composition for decreasing loss of and/or improving gait, locomotion, and/or balance in a subject in need thereof comprises α-ketoglutarate salt. In some embodiments, the composition for decreasing loss of and/or improving gait, locomotion, and/or balance in a subject in need thereof consists essentially of α-ketoglutarate salt.

In some embodiments, the composition for decreasing loss of and/or improving gait, locomotion, and/or balance in a subject in need thereof comprises an ester of α-ketoglutarate.

In some embodiments, the composition for decreasing loss of and/or improving gait, locomotion, and/or balance in a subject in need thereof comprises vitamin A. In some embodiments, the composition comprises vitamin A and Ca-AKG. In some embodiments, the composition for decreasing loss of and/or improving gait, locomotion, and/or balance in a subject in need thereof comprises Vitamin D. In some embodiments, the composition comprises vitamin D and Ca-AKG. In some embodiments, the composition for decreasing loss of and/or improving gait, locomotion, and/or balance in a subject in need thereof comprises Vitamin E. In some embodiments, the composition comprises vitamin E and Ca-AKG.

In some embodiments, the composition for decreasing loss of and/or improving gait, locomotion, and/or balance in a subject in need thereof comprises a compound which targets mitochondria.

In some embodiments, the composition for decreasing loss of and/or improving gait, locomotion, and/or balance in a subject in need thereof comprises a branched-chain amino acid.

In some embodiments, the composition for decreasing loss of and/or improving gait, locomotion, and/or balance in a subject in need thereof comprises an alpha-keto acid. In some embodiments, the composition for decreasing loss of and/or improving gait, locomotion, and/or balance in a subject in need thereof comprises a beta-hydroxy acid.

In some embodiments, the composition for decreasing loss of and/or improving gait, locomotion, and/or balance in a subject in need thereof comprises a tyrosine kinase inhibitor.

In some embodiments, the composition for decreasing loss of and/or improving gait, locomotion, and/or balance in a subject in need thereof comprises a senolytic agent. In some embodiments, the senolytic agent is combined with AKG. In some embodiments, the senolytic agent is combined with CaAKG.

### Exercise Endurance

In some embodiments, the disclosure provides methods for decreasing loss of and/or increasing exercise endurance, exercise efficiency, and/or metabolism in a subject in need thereof using the active agents or compositions thereof described herein. In some embodiments, the disclosure provides methods for decreasing loss of and/or increasing exercise endurance, exercise efficiency, and/or metabolism in a subject in need thereof using the active agents or compositions thereof described herein. In some embodiments, the disclosure provides methods for decreasing loss of and/or increasing exercise endurance, exercise efficiency, and/or metabolism in a subject in need thereof using the active agents or compositions thereof described herein.

In some embodiments, the composition for decreasing loss of and/or increasing exercise endurance, exercise efficiency, and/or metabolism in a subject in need thereof comprises alpha-ketoglutarate (AKG). In some embodiments, the composition for decreasing loss of and/or increasing exercise endurance, exercise efficiency, and/or metabolism in a subject in need thereof consists essentially of alpha-ketoglutarate (AKG).

In some embodiments, the composition for decreasing loss of and/or increasing exercise endurance, exercise efficiency, and/or metabolism in a subject in need thereof comprises the biosynthetic precursor to NAD+ and AKG. In some embodiments, the composition for decreasing loss of and/or increasing exercise endurance, exercise efficiency, and/or metabolism in a subject in need thereof comprises the biosynthetic precursor to NAD+ and Ca-AKG.

In some embodiments, the composition for decreasing loss of and/or increasing exercise endurance, exercise efficiency, and/or metabolism in a subject in need thereof comprises the biosynthetic precursor to NAD+, AKG and pterostilbene.

In some embodiments, the composition for decreasing loss of and/or increasing exercise endurance, exercise efficiency, and/or metabolism in a subject in need thereof comprises α-ketoglutarate salt. In some embodiments, the composition for decreasing loss of and/or increasing exercise endurance, exercise efficiency, and/or metabolism in a subject in need thereof consists essentially of α-ketoglutarate salt.

In some embodiments, the composition for decreasing loss of and/or increasing exercise endurance, exercise efficiency, and/or metabolism in a subject in need thereof comprises an ester of α-ketoglutarate.

In some embodiments, the composition for decreasing loss of and/or increasing exercise endurance, exercise efficiency, and/or metabolism in a subject in need thereof comprises vitamin A. In some embodiments, the composition comprises vitamin A and Ca-AKG. In some embodiments, the composition for decreasing loss of and/or increasing exercise endurance, exercise efficiency, and/or metabolism in a subject in need thereof comprises Vitamin D. In some embodiments, the composition comprises vitamin D and Ca-AKG. In some embodiments, the composition for decreasing loss of and/or increasing exercise endurance, exercise efficiency, and/or metabolism in a subject in need thereof comprises Vitamin E. In some embodiments, the composition comprises vitamin E and Ca-AKG.

In some embodiments, the composition for decreasing loss of and/or increasing exercise endurance, exercise efficiency, and/or metabolism in a subject in need thereof comprises a compound which targets mitochondria.

In some embodiments, the composition for decreasing loss of and/or increasing exercise endurance, exercise efficiency, and/or metabolism in a subject in need thereof comprises a branched-chain amino acid.

In some embodiments, the composition for decreasing loss of and/or increasing exercise endurance, exercise efficiency, and/or metabolism in a subject in need thereof comprises an alpha-keto acid. In some embodiments, the composition for decreasing loss of and/or increasing exercise endurance, exercise efficiency, and/or metabolism in a subject in need thereof comprises a beta-hydroxy acid.

In some embodiments, the composition for decreasing loss of and/or increasing exercise endurance, exercise efficiency, and/or metabolism in a subject in need thereof comprises a tyrosine kinase inhibitor.

In some embodiments, the composition for decreasing loss of and/or increasing exercise endurance, exercise efficiency, and/or metabolism in a subject in need thereof comprises a senolytic agent. In some embodiments, the senolytic agent is combined with AKG. In some embodiments, the senolytic agent is combined with CaAKG.

### Sarcopenia

In some embodiments, the disclosure provides methods for treating sarcopenia in a subject in need thereof using the active agents or compositions thereof described herein. In some embodiments, the disclosure provides methods for treating sarcopenia in a subject in need thereof using the active agents or compositions thereof described herein. In some embodiments, the disclosure provides methods for treating sarcopenia in a subject in need thereof using the active agents or compositions thereof described herein.

In some embodiments, the composition for treating sarcopenia in a subject in need thereof comprises alpha-ketoglutarate (AKG). In some embodiments, the composition for treating sarcopenia in a subject in need thereof consists essentially of alpha-ketoglutarate (AKG).

In some embodiments, the composition for treating sarcopenia in a subject in need thereof comprises the biosynthetic precursor to NAD+ and AKG. In some embodiments, the composition for treating sarcopenia in a subject in need thereof comprises the biosynthetic precursor to NAD+ and Ca-AKG.

In some embodiments, the composition for treating sarcopenia in a subject in need thereof comprises the biosynthetic precursor to NAD+, AKG and pterostilbene.

In some embodiments, the composition for treating sarcopenia in a subject in need thereof comprises α-ketoglutarate salt. In some embodiments, the composition for treating sarcopenia in a subject in need thereof consists essentially of α-ketoglutarate salt.

In some embodiments, the composition for treating sarcopenia in a subject in need thereof comprises an ester of α-ketoglutarate.

In some embodiments, the composition for treating sarcopenia in a subject in need thereof comprises vitamin A. In some embodiments, the composition comprises vitamin A and Ca-AKG. In some embodiments, the composition for treating sarcopenia in a subject in need thereof comprises Vitamin D. In some embodiments, the composition comprises vitamin D and Ca-AKG. In some embodiments, the composition for treating sarcopenia in a subject in need thereof comprises Vitamin E. In some embodiments, the composition comprises vitamin E and Ca-AKG.

In some embodiments, the composition for treating sarcopenia in a subject in need thereof comprises a compound which targets mitochondria.

In some embodiments, the composition for treating sarcopenia in a subject in need thereof comprises a branched-chain amino acid.

In some embodiments, the composition for treating sarcopenia in a subject in need thereof comprises an alpha-keto acid. In some embodiments, the composition for treating sarcopenia in a subject in need thereof comprises a beta-hydroxy acid.

In some embodiments, the composition for treating sarcopenia in a subject in need thereof comprises a tyrosine kinase inhibitor.

In some embodiments, the composition for treating sarcopenia in a subject in need thereof comprises a senolytic agent. In some embodiments, the senolytic agent is combined with AKG. In some embodiments, the senolytic agent is combined with CaAKG.

### Collagen Synthesis

In some embodiments, the disclosure provides methods for inducing, promoting, or increasing collagen synthesis in a subject in need thereof using the active agents or compositions thereof described herein. In some embodiments, the disclosure provides methods for inducing, promoting, or increasing collagen synthesis in a subject in need thereof using the active agents or compositions thereof described herein. In some embodiments, the disclosure provides methods for inducing, promoting, or increasing collagen synthesis in a subject in need thereof using the active agents or compositions thereof described herein.

In some embodiments, the composition for inducing, promoting, or increasing collagen synthesis in a subject in need thereof comprises alpha-ketoglutarate (AKG). In some embodiments, the composition for inducing, promoting, or increasing collagen synthesis in a subject in need thereof consists essentially of alpha-ketoglutarate (AKG).

In some embodiments, the composition for inducing, promoting, or increasing collagen synthesis in a subject in need thereof comprises the biosynthetic precursor to NAD+ and AKG. In some embodiments, the composition for inducing, promoting, or increasing collagen synthesis in a subject in need thereof comprises the biosynthetic precursor to NAD+ and Ca-AKG.

In some embodiments, the composition for inducing, promoting, or increasing collagen synthesis in a subject in need thereof comprises the biosynthetic precursor to NAD+, AKG and pterostilbene.

In some embodiments, the composition for inducing, promoting, or increasing collagen synthesis in a subject in need thereof comprises α-ketoglutarate salt. In some embodiments, the composition for inducing, promoting, or increasing collagen synthesis in a subject in need thereof consists essentially of α-ketoglutarate salt.

In some embodiments, the composition for inducing, promoting, or increasing collagen synthesis in a subject in need thereof comprises an ester of α-ketoglutarate.

In some embodiments, the composition for inducing, promoting, or increasing collagen synthesis in a subject in need thereof comprises vitamin A. In some embodiments, the composition comprises vitamin A and Ca-AKG. In some embodiments, the composition for inducing, promoting, or increasing collagen synthesis in a subject in need thereof comprises Vitamin D. In some embodiments, the composition comprises vitamin D and Ca-AKG. In some embodiments, the composition for inducing, promoting, or increasing collagen synthesis in a subject in need thereof comprises Vitamin E. In some embodiments, the composition comprises vitamin E and Ca-AKG.

In some embodiments, the composition for inducing, promoting, or increasing collagen synthesis in a subject in need thereof comprises a compound which targets mitochondria.

In some embodiments, the composition for inducing, promoting, or increasing collagen synthesis in a subject in need thereof comprises a branched-chain amino acid.

In some embodiments, the composition for inducing, promoting, or increasing collagen synthesis in a subject in need thereof comprises an alpha-keto acid. In some embodiments, the composition for inducing, promoting, or increasing collagen synthesis in a subject in need thereof comprises a beta-hydroxy acid.

In some embodiments, the composition for inducing, promoting, or increasing collagen synthesis in a subject in need thereof comprises a tyrosine kinase inhibitor.

In some embodiments, the composition for inducing, promoting, or increasing collagen synthesis in a subject in need thereof comprises a senolytic agent. In some embodiments, the senolytic agent is combined with AKG. In some embodiments, the senolytic agent is combined with CaAKG.

### Altering Senescence-Associated Secretory Phenotype (SASP)

In an aspect, the disclosure provides methods of altering senescence-associated secretory phenotype (SASP) using the active agents or compositions thereof described herein. In some embodiments, the methods disclosed herein alter SASP of a senescent cell in a subject in need thereof. A “senescent cell” is generally thought to be derived from a cell type that typically replicates, but as a result of aging or other event that causes a change in cell state, can no longer replicate. The nucleus of senescent cells is often characterized by senescence-associated heterochromatin foci and DNA segments with chromatin alterations reinforcing senescence. It remains metabolically active and commonly adopts a senescence associated secretory phenotype (SASP). The SASP can include a combination of inflammatory cytokines, growth factors, and proteases. Without being bound by a particulary theory, the SASP secreted by senescent cells can contribute to the onset and progression of age-related diseases.

In an aspect, the disclosure provides methods of altering senescence-associated secretory phenotype (SASP) using the active agents or compositions thereof described herein. In some aspects, the composition comprises two or more active agents as described herein.

In some embodiments, the composition for altering SASP in a subject in need thereof comprises alpha-ketoglutarate (AKG). In some embodiments, the composition for altering SASP in a subject in need thereof consists essentially of alpha-ketoglutarate (AKG).

In some embodiments, the composition for altering SASP in a subject in need thereof comprises the biosynthetic precursor to NAD+ and AKG. In some embodiments, the composition for altering SASP in a subject in need thereof comprises the biosynthetic precursor to NAD+ and Ca-AKG.

In some embodiments, the composition for altering SASP in a subject in need thereof comprises the biosynthetic precursor to NAD+, AKG and pterostilbene.

In some embodiments, the composition for altering SASP in a subject in need thereof comprises α-ketoglutarate salt. In some embodiments, the composition for altering SASP in a subject in need thereof consists essentially of α-ketoglutarate salt.

In some embodiments, the composition for altering SASP in a subject in need thereof comprises an ester of α-ketoglutarate.

In some embodiments, the composition for altering SASP in a subject in need thereof comprises vitamin A. In some embodiments, the composition comprises vitamin A and Ca-AKG. In some embodiments, the composition for altering SASP in a subject in need thereof comprises Vitamin D. In some embodiments, the composition comprises vitamin D and Ca-AKG. In some embodiments, the composition for altering SASP in a subject in need thereof comprises Vitamin E. In some embodiments, the composition comprises vitamin E and Ca-AKG.

In some embodiments, the composition for altering SASP in a subject in need thereof comprises a compound which targets mitochondria.

In some embodiments, the composition for altering SASP in a subject in need thereof comprises a branched-chain amino acid.

In some embodiments, the composition for altering SASP in a subject in need thereof comprises an alpha-keto acid. In some embodiments, the composition for altering SASP in a subject in need thereof comprises a beta-hydroxy acid.

In some embodiments, the composition for altering SASP in a subject in need thereof comprises a tyrosine kinase inhibitor.

In some embodiments, the composition for altering SASP in a subject in need thereof comprises a senolytic agent. In some embodiments, the senolytic agent is combined with AKG. In some embodiments, the senolytic agent is combined with CaAKG.

In some embodiments, the methods disclosed herein alter SASP of a senescent cell in a subject in need thereof. In some embodiments, the altering of SASP is not in an injured cell. In some embodiments, the altering of SASP is not in a proliferating cell. In some embodiments, the methods comprise administering to the subject a composition comprising AKG, wherein the AKG delays SASP. In some embodiments, the methods comprise administering to the subject a composition consisting essentially of AKG, wherein the AKG delays SASP. In some embodiments, the methods comprise administering to the subject a composition comprising AKG, wherein the AKG reduces SASP. In some embodiments, the methods comprise administering to the subject a composition consisting essentially of AKG, wherein the AKG reduces SASP.

In some embodiments, the alteration of SASP is delayed for at least 1 month after administration of the composition to the subjects. In some embodiments, the alteration of SASP is delayed for at least 2 months after administration of the composition to the subjects. In some embodiments, the alteration of SASP is delayed for at least 3 months after administration of the composition to the subjects. In some embodiments, the alteration of SASP is delayed for at least 4 months after administration of the composition to the subjects. In some embodiments, the alteration of SASP is delayed for at least 5 months after administration of the composition to the subjects. In some embodiments, the alteration of SASP is delayed for at least 6 months after administration of the composition to the subjects. In some embodiments, the alteration of SASP is delayed for at least 7 months after administration of the composition to the subjects. In some embodiments, the alteration of SASP is delayed for at least 8 months after administration of the composition to the subjects. In some embodiments, the alteration of SASP is delayed for at least 9 months after administration of the composition to the subjects. In some embodiments, the alteration of SASP is delayed for at least 10 months after administration of the composition to the subjects. In some embodiments, the alteration of SASP is delayed for at least 11 months after administration of the composition to the subjects. In some embodiments, the alteration of SASP is delayed for at least 12 months after administration of the composition to the subjects. In some embodiments, the alteration of SASP is delayed for at least 18 months after administration of the composition to the subjects. In some embodiments, the alteration of SASP is delayed for at least 24 months after administration of the composition to the subjects. In some embodiments, the alteration of SASP is delayed for at least 30 months after administration of the composition to the subjects. In some embodiments, the alteration of SASP is delayed for at least 36 months after administration of the composition to the subjects. In some embodiments, the composition reduces the SASP relative to a pretreatment value of the SASP. In some embodiments, the SASP is decreased at least 5%, 10%, 15%, 20%, 25%, 33%, 40%, 45%, 50%, 66%, 75%, or 100% relative to the pretreatment value.

In some embodiments, altering SASP comprises altering secretion of at least one, at least two, at least three, at least four, or at least five SASP factors. In some embodiments, altering SASP comprises altering secretion of at least one SASP factor. In some embodiments, altering SASP comprises altering secretion of at least two SASP factors. In some embodiments, altering SASP comprises altering secretion of at least three SASP factors. In some embodiments, altering SASP comprises altering secretion of at least four SASP factors. In some embodiments, altering SASP comprises altering secretion of at least five SASP factors. In some embodiments, altering secretion comprises reducing secretion of at least one, at least two, at least three, at least four, or at least five SASP factors. In some embodiments, altering secretion comprises delaying secretion of at least one, at least two, at least three, at least four, or at least five SASP factors.

In some embodiments, the SASP factors comprise an interleukin (IL), a chemokine, an inflammatory factor, a growth factor, a protease, an extracellular matrix component, or a combination thereof. In some embodiments, the SASP factors comprises IL-1α, IL-1β, IL-2, IL-3, IL-4, IL-5, IL-6, IL-7, IL-8, IL-9, IL-10, IL-12, IL-13, IL-15, IL-17, IP-10, KC, LIX, GRO-α, GRO-β, GRO-γ, MCP-2, MCP-4, MIP-1α, MIP-1β, MIP-2, MIP-3α, HCC-4, Eotaxin-3, TNF-α, GM-CSE, MIF, amphiregulin, epiregulin, heregulin, EGF, FGF, HGF, KGF, VEGF, angiogenin, SCF, SDF-1, PIGF, IGFBP-2, IGFBP-3, IGFBP-4, IGFBP-6, IGFBP-7, MMP-1, MMP-3, MMP-10, MMP-12, MMP-13, MMP-14, TIMP-1, TIMP-2, PAI-1, PAI-2, tPA, uPA, cathepsin B, ICAM-1, ICAM-3, OPG, sTNFRI, TRAIL-R3, Fas, sTNFRII, Fas, uPAR, SGP130, EGF-R, PGE-2, nitric oxide, fibronectin, Eoxtaxin, G-CSF, GM-CSF, M-CSF, IFNγ, MIG, RANTES, CCL2, CXCL1, or a combination thereof. In some embodiments, the SASP factors comprises Eoxtaxin, G-CSF, GM-CSF, IFNγ, IL-1α, IL-1β, IL-2, IL-3, IL-4, IL-5, IL-6, IL-7, IL-8, IL-9, IL-10, IL-12, IL-13, IL-15, IL-17, IP-10, KC, LIX, M-CSF, MIG, MIP-1α, MIP-1β, MIP-2, RANTES, TNF-α, VEGF, CCL2, CXCL1, MMP-3, or a combination thereof. In some embodiments, the SASP factors comprise IL-1β, IL-3, IL-6, IL-7, MIP-1β, TNF-α, CCL2, MMP3, or a combination thereof.

In some embodiments, altering SASP does not affect formation of senescent cells. In some embodiments, altering SASP does not affect number of senescent cells. In some embodiments, altering SASP does not include killing senescent cells. In some embodiments, altering SASP does not induce apoptosis of senescent cells. In some embodiments, prior to administering the composition to the subject, a biological sample is collected from the subject. In some embodiments, the biological sample is selected from the group consisting of blood, plasma, saliva, urine, tissue, and cerebrospinal fluid. In some embodiments, the biological sample is split into a control biological sample and a test biological sample wherein the composition is applied to the test biological sample after the biological sample is split.

In some embodiments, a senescence stimulus is applied to the control biological sample and the test biological sample before the composition is applied, and the formation of senescent cells is measured in the control biological sample and the test biological sample. In some embodiments, the formation, number, killing and/or apoptosis of senescent cells is measured using at least one, at least two, at least three, at least four, or at least five senescent markers. In some embodiments, the senescence biomarker is a molecule that is part of SASP. In some embodiments, the molecule that is part of SASP includes, but is not limited to GM-CSF, GROα, GROα,β,β, IGFBP-7, IL-1α, IL-6, IL-7, IL-8, MCP-1, MCP-2, MIP-1α, MMP-1, MMP-10, MMP-3, Amphiregulin, ENA-78, Eotaxin-3, GCP-2, GITR, HGF, ICAM-1, IGFBP-2, IGFBP-4, IGFBP-5, IGFBP-6, IL-13, IL-1β, MCP-4, MIF, MIP-3α, MMP-12, MMP-13, MMP-14, NAP2, Oncostatin M, osteoprotegerin, PIGF, RANTES, sgp130, TIMP-2, TRAIL-R3, Acrp30, angiogenin, Axl, bFGF, BLC, BTC, CTACK, EGF-R, Fas, FGF-7, G-CSF, GDNF, HCC-4, 1-309, IFN-γ, IGFBP-1, IGFBP-3, IL-1 R1, IL-11, IL-15, IL-2R-α, IL-6 R, I-TAC, Leptin, LIF, MMP-2, MSP-a, PAI-1, PAI-2, PDGF-BB, SCF, SDF-1, sTNF RI, sTNF RII, Thrombopoietin, TIMP-1, tPA, uPA, uPAR, VEGF, MCP-3, IGF-1, TGF-↑3, MIP-1-delta, IL-4, FGF-7, PDGF-BB, IL-16, BMP-4, MDC, MCP-4, IL-10, TIMP-1, Fit-3 Ligand, ICAM-1, Axl, CNTF, INF-γ, EGF, BMP-6. In some embodiments, the senescence biomarker is selected from the group consisting of senescence-associated β-galactosidase, mmp-3, IL-6, p21, p16, and p53. In some embodiments, the senescence biomarker is senescence-associated β-galactosidase. In some embodiments, at least one, at least two, at least three, at least four, or at least five senescent markers comprises p16^{INK4a}. In some embodiments, the senescence biomarker is senescence-associated p16I^{NK4a}. In some embodiments, at least one, at least two, at least three, at least four, or at least five senescent markers comprises p21^{WAF1}. In some embodiments, the senescence biomarker is senescence-associated p21^{WAF1}. In some embodiments, the formation of senescent cells is characterized by senescence associated heterochromatin foci (SAHF) or DNA segments with chromatin alteration reinforcing senescence (DNA-SCARS). In some embodiments, the test biological sample has reduced senescence biomarkers compared to the control biological sample which indicates a reduction in the formation of senescent cells in the test biological sample.

In some embodiments, the amount of the SASP-altering agent is at least 5 mg/kg, at least 10 mg/kg, at least 15 mg/kg, at least 16 mg/kg, at least 16.1 mg/kg, at least 16.2 mg/kg, at least 16.2 mg/kg, at least 16.4 mg/kg, at least 16.5 mg/kg, at least 16.6 mg/kg, at least 16.7 mg/kg, at least 16.8 mg/kg, at least 16.9 mg/kg, at least 17 mg/kg, at least 18 mg/kg, at least 19 mg/kg, at least 20 mg/kg, at least 21 mg/kg, at least 22 mg/kg, at least 23 mg/kg, at least 24 mg/kg, at least 25 mg/kg, at least 28 mg/kg, at least 30 mg/kg, or at least 33 mg/kg.

In some embodiments, the senescence stimulus is selected from the group consisting of chemotherapy treatment, irradiation, oxidative stress, and aging.

In some embodiments, the subject is pre-screened for plasma AKG level. In some embodiments, the subject has reduced plasma AKG level. In some embodiments, the subject has at least 6 fold reduction in plasma AKG level compared to plasma AKG level in a control sample. In some embodiments, the subject has at least 7 fold reduction in plasma AKG level compared to plasma AKG level in a control sample. In some embodiments, the subject has at least 8 fold reduction in plasma AKG level compared to plasma AKG level in a control sample. In some embodiments, the subject has at least 9 fold reduction in plasma AKG level compared to plasma AKG level in a control sample. In some embodiments, the subject has at least 10 fold reduction in plasma AKG level compared to plasma AKG level in a control sample. In some embodiments, the control sample is a serum sample from a control subject who is at least 2, at least 3, at least 4, at least 5, at least 8, or at least 10 years younger than the subject.

In some embodiments, the subject is pre-screened for a DNA methylation profile. In some embodiments, the DNA methylation profile of the subject is comparable to a DNA methylation profile of a control subject. In some embodiments, the control subject is a subject administered a placebo. In some embodiments, the control subject is at least of same age as the subject. In some embodiments, the composition alters the DNA methylation profile of the subject. In some embodiments, the altered DNA methylation profile of the subject is comparable to a second control subject who is at least 2 years younger than the subject. In some embodiments, the altered DNA methylation profile of the subject is comparable to a second control subject who is at least 3 years younger than the subject. In some embodiments, the altered DNA methylation profile of the subject is comparable to a second control subject who is at least 4 years younger than the subject. In some embodiments, the altered DNA methylation profile of the subject is comparable to a second control subject who is at least 5 years younger than the subject. In some embodiments, the altered DNA methylation profile of the subject is comparable to a second control subject who is at least 6 years younger than the subject. In some embodiments, the altered DNA methylation profile of the subject is comparable to a second control subject who is at least 7 years younger than the subject. In some embodiments, the altered DNA methylation profile of the subject is comparable to a second control subject who is at least 8 years younger than the subject. In some embodiments, the altered DNA methylation profile of the subject is comparable to a second control subject who is at least 9 years younger than the subject. In some embodiments, the altered DNA methylation profile of the subject is comparable to a second control subject who is at least 10 years younger than the subject.

In some embodiments, the composition further delays or reverses an age-related phenotype. In some embodiments, the age-related phenotype comprises sarcopenia, impaired cognition, or age-related epigenetic changes. In some embodiments, epigenetic changes comprise changes in DNA methylation profile. In some embodiments, the composition further induces or promotes collagen synthesis.

As described herein, the compositions and methods are useful for treating diseases and conditions, including but not limited to treating, delaying onset, or delaying progression of frailty, extending healthspan, compressing morbidity, helping to maintain health, maintaining hair density, maintaining hair pigmentation, re-growing hair, altering SASP, improving gait and balance, increasing exercise endurance, increasing exercise efficiency and metabolism, treating sarcopenia, and increasing collagen synthesis in a subject. Additional embodiments of those compositions and methods are described hereinbelow.

In each of the embodiments described herein, the composition may comprise AKG and niacin. In some embodiments, the composition comprises AKG and nicotinamide. In some embodiments, the composition comprises AKG and NR. In some embodiments, the composition comprises AKG and NMN. In some embodiments, the composition comprises Ca-AKG and niacin. In some embodiments, the composition comprises Ca-AKG and nicotinamide. In some embodiments, the composition comprises Ca-AKG and NR. In some embodiments, the composition comprises Ca-AKG and NMN. In some embodiments, the composition comprises NAD+ or NADH and AKG. In some embodiments, the composition comprises NAD+ or NADH with CaAKG.

In each of the embodiments described herein, the composition may comprise AKG, niacin, and pterostilbene. In some embodiments, the composition comprises AKG, nicotinamide, and pterostilbene. In some embodiments, the composition comprises AKG, NR, and pterostilbene. In some embodiments, the composition comprises AKG, NMN, and pterostilbene. In some embodiments, the composition comprises the biosynthetic precursor to NAD+, CaAKG and pterostilbene. In some embodiments, the composition comprises CaAKG, niacin, and pterostilbene. In some embodiments, the composition comprises CaAKG, nicotinamide, and pterostilbene. In some embodiments, the composition comprises CaAKG, NR, and pterostilbene. In some embodiments, the composition comprises CaAKG, NMN, and pterostilbene. In some embodiments, the composition comprises NAD+ or NADH with AKG and pterostilbene. In some embodiments, the composition comprises NAD+ or NADH with CaAKG and pterostilbene.

In each of the embodiments described herein, α-ketoglutarate may be provided as a calcium salt (Ca-AKG). In some embodiments, calcium α-ketoglutarate can be a hydrate calcium α-ketoglutarate. In some embodiments, calcium α-ketoglutarate can be a mono-hydrate calcium α-ketoglutarate. In some embodiments, calcium α-ketoglutarate can be hemi-hydrate calcium α-ketoglutarate. In some embodiments, calcium α-ketoglutarate can be anhydrous calcium α-ketoglutarate.

In each of the embodiments described herein, the ester of α-ketoglutarate may be a methyl ester of α-ketoglutarate. In some embodiments, the ester of α-ketoglutarate is a dimethyl ester of α-ketoglutarate. In some embodiments, the ester of α-ketoglutarate is an ethyl ester of α-ketoglutarate. In some embodiments, the ester of α-ketoglutarate is a diethyl ester of α-ketoglutarate.

In each of the embodiments described herein, the compositions may comprise vitamin A combined with one or more of the active agents disclosed herein (e.g. AKG or Ca-AKG, vitamin D, NR). As disclosed herein, vitamin A can also be provided as retinoic acid (RA), retinyl palmitate (RP), or retinyl acetate. In each of the embodiments described herein, the composition may comprise retinol ester, retinol, retinal, retinoic acid, or retinoic acid salts with pharmaceutically acceptable cations as described previously.

In each of the embodiments described herein, the composition may comprise vitamin A and AKG. In some embodiments, the composition comprises vitamin A and NR.

In each of the embodiments described herein, the composition may comprise the alpha-, beta-, or gamma-carotene forms represented in formulas 8-10. In some embodiments, the composition comprises retinol ester. In some embodiments, the composition comprises retinol. In some embodiments, the composition comprises retinoic acid.

In each of the embodiments described herein, the composition may comprise vitamin D in combination with one or more of the active agents disclosed herein (AKG or Ca-AKG, vitamin A, NR). In some embodiments, the composition comprises vitamin D₂ or ergocalciferol, vitamin D₃ or cholecalciferol, vitamin D₄ or 22-dihydroergocalciferol, vitamin D₅ or sitocalciferol, and vitamin D₁ or a 1:1 mixture of ergocalciferol with lumisterol. In some embodiments, the composition comprises vitamin D and Ca-AKG. In some embodiments, the composition comprises vitamin D and AKG. In some embodiments, the composition comprises vitamin D and NR.

In each of the embodiments described herein, the composition may comprise vitamin E in combination with one or more of the active agents disclosed herein (AKG or Ca-AKG, vitamin A, NR). In some embodiments, the composition comprises tocopherol, alpha-tocopherol, beta-tocopherol, gamma-tocopherol, and delta-tocopherol. In some embodiments, the composition comprises tocotrienol. In some embodiments, the composition comprises alpha-tocotrienol. In some embodiments, the composition comprises beta-tocotrienol. In some embodiments, the composition comprises gamma-tocotrienol. In some embodiments, the composition comprises delta-tocotrienol.

In some embodiments, the composition comprises vitamin E and Ca-AKG. In some embodiments, the composition comprises vitamin E and AKG. In some embodiments, the composition comprises vitamin E and NR.

In each of the embodiments described herein, the composition may comprise a compound which targets mitochondria. In some embodiments, the compound which targets mitochondria includes, but is not limited to alpha-lipoic acid, L-carnitine, coenzyme Q10 (CoQ10 (ubiquinone), nicotinamide adenine dinucleotide (NADH), vitamin E, vitamin C (ascorbic acid), pantothenic acid (Vitamin B5), or astaxanthin. In some embodiments, the NADH is microencapsulated NADH. In some embodiments, the vitamin E is alpha-tocopherol acetate. In some embodiments, the composition comprises alpha-lipoic acid. In some embodiments, the composition comprises L-carnitine. In some embodiments, the composition comprises coenzyme Q10 (CoQ10 (ubiquinone). In some embodiments, the composition comprises NADH. In some embodiments, the composition comprises vitamin E. In some embodiments, the composition comprises vitamin C. In some embodiments, the composition comprises pantothenic acid. In some embodiments, the composition comprises astaxanthin. In some embodiments, the compound which targets mitochondria is combined with AKG. In some embodiments, the composition comprises AKG and alpha-lipoic acid. In some embodiments, the composition comprises AKG and L-carnitine. In some embodiments, the composition comprises AKG and coenzyme Q10. In some embodiments, the composition comprises AKG and NADH. In some embodiments, the composition comprises AKG and vitamin E. In some embodiments, the composition comprises AKG and vitamin C. In some embodiments, the composition comprises AKG and pantothenic acid. In some embodiments, the composition comprises AKG and astaxanthin. In some embodiments, the compound which targets mitochondria is combined with CaAKG. In some embodiments, the composition comprises CaAKG and alpha-lipoic acid. In some embodiments, the composition comprises CaAKG and L-carnitine. In some embodiments, the composition comprises CaAKG and coenzyme Q10. In some embodiments, the composition comprises CaAKG and NADH. In some embodiments, the composition comprises CaAKG and vitamin E. In some embodiments, the composition comprises CaAKG and vitamin C. In some embodiments, the composition comprises CaAKG and pantothenic acid. In some embodiments, the composition comprises CaAKG and astaxanthin.

In each of the embodiments described herein, the composition may comprise L-leucine, L-isoleucine, or L-valine. In some embodiments, the composition comprises L-leucine. In some embodiments, the composition comprises L-isoleucine. In some embodiments, the composition comprises L-valine. In some embodiments, the branched-chain amino acid is combined with AKG. In some embodiments, the composition comprises AKG and L-leucine. In some embodiments, the composition comprises AKG and L-isoleucine. In some embodiments, the composition comprises AKG and L-valine. In some embodiments, the branched-chain amino acid is combined with CaAKG. In some embodiments, the composition comprises CaAKG and L-leucine. In some embodiments, the composition comprises CaAKG and L-isoleucine. In some embodiments, the composition comprises CaAKG and L-valine.

In each of the embodiments described herein, the composition may comprise an alpha-keto acid. In some embodiments, the alpha-keto acid includes, but is not limited to, 3-methyl-2-oxobutyrate, 3-methyl-2-oxovalerate , or 4-methyl-2-oxovalerate (α-ketoisocaproic acid). In some embodiments, the alpha-keto acid is 3-methyl-2-oxobutyrate. In some embodiments, the alpha-keto acid is 3-methyl-2-oxovalerate. In some embodiments, the alpha-keto acid is 4-methyl-2-oxovalerate. In some embodiments, the alpha-keto acid is combined with AKG. In some embodiments, the composition comprises AKG and 3-methyl-2-oxobutyrate. In some embodiments, the composition comprises AKG and 3-methyl-2-oxovalerate. In some embodiments, the composition comprises AKG and 4-methyl-2-oxovalerate (α-ketoisocaproic acid). In some embodiments, the alpha-keto acid is combined with CaAKG. In some embodiments, the composition comprises CaAKG and 3-methyl-2-oxobutyrate. In some embodiments, the composition comprises CaAKG and 3-methyl-2-oxovalerate. In some embodiments, the composition comprises CaAKG and 4-methyl-2-oxovalerate (α-ketoisocaproic acid). In some embodiments, the composition comprises alpha-ketobutyrate. In some embodiments, the composition comprises alpha-ketobutyrate and AKG. In some embodiments, the composition comprises alpha-ketobutyrate and CaAKG.

In each of the embodiments described herein, the composition may comprise a beta-hydroxy acid. In some embodiments, the beta-hydroxy acid is beta-hydroxy-beta-methylbutyrate. In some embodiments, the beta-hydroxy acid is combined with AKG. In some embodiments, the composition comprises AKG and beta-hydroxy-beta-methylbutyrate. In some embodiments, the beta-hydroxy acid is combined with CaAKG. In some embodiments, the composition comprises CaAKG and beta-hydroxy-beta-methylbutyrate. In some embodiments, the composition comprises D-beta-hydroxybutyrate. In some embodiments, the composition comprises D-beta-hydroxybutyrate and AKG. In some embodiments, the composition D-beta-hydroxybutyrate and CaAKG.

In some embodiments, the composition comprises D-beta-hydroxybutyrate, alpha-ketobutyrate, and AKG. In some embodiments, the composition comprises D-beta-hydroxybutyrate, alpha-ketobutyrate, and CaAKG.

In each of the embodiments described herein, the composition may comprise a tyrosine kinase inhibitor. In some embodiments, the tyrosine kinase inhibitor includes, but is not limited to, piceatannol, erlotinib, gefitinib, sorafenib, sunitinib, genistein, curcumin, carnosol, ursolic acid, daidzein, luteolin, quercetin, or senolytic agent. In some embodiments, the tyrosine kinase inhibitor is piceatannol. In some embodiments, the tyrosine kinase inhibitor is erlotinib. In some embodiments, the tyrosine kinase inhibitor is gefitinib. In some embodiments, the tyrosine kinase inhibitor is sorafenib. In some embodiments, the tyrosine kinase inhibitor is sunitinib. In some embodiments, the tyrosine kinase inhibitor is genistein. In some embodiments, the tyrosine kinase inhibitor is curcumin. In some embodiments, the tyrosine kinase inhibitor is carnosol. In some embodiments, the tyrosine kinase inhibitor is ursolic acid. In some embodiments, the tyrosine kinase inhibitor is daidzein. In some embodiments, the tyrosine kinase inhibitor is luteolin. In some embodiments, the tyrosine kinase inhibitor is quercetin. In some embodiments, the tyrosine kinase inhibitor is senolytic agent.

In some embodiments, the tyrosine kinase inhibitor is combined with AKG. In some embodiments, the composition comprises AKG and piceatannol. In some embodiments, the composition comprises AKG and erlotinib. In some embodiments, the composition comprises AKG and gefitinib. In some embodiments, the composition comprises AKG and sorafenib. In some embodiments, the composition comprises AKG and sunitinib. In some embodiments, the composition comprises AKG and genistein. In some embodiments, the composition comprises AKG and curcumin. In some embodiments, the composition comprises AKG and carnosol. In some embodiments, the composition comprises AKG and ursolic acid. In some embodiments, the composition comprises AKG and daidzein. In some embodiments, the composition comprises AKG and luteolin. In some embodiments, the composition comprises AKG and quercetin. In some embodiments, the composition comprises AKG and senolytic agent.

In some embodiments, the tyrosine kinase inhibitor is combined with CaAKG. In some embodiments, the composition comprises CaAKG and piceatannol. In some embodiments, the composition comprises CaAKG and erlotinib. In some embodiments, the composition comprises CaAKG and gefitinib. In some embodiments, the composition comprises CaAKG and sorafenib. In some embodiments, the composition comprises CaAKG and sunitinib. In some embodiments, the composition comprises CaAKG and genistein. In some embodiments, the composition comprises CaAKG and curcumin. In some embodiments, the composition comprises CaAKG and carnosol. In some embodiments, the composition comprises CaAKG and ursolic acid. In some embodiments, the composition comprises CaAKG and daidzein. In some embodiments, the composition comprises CaAKG and luteolin. In some embodiments, the composition comprises CaAKG and quercetin. In some embodiments, the composition comprises CaAKG and senolytic agent. In each of the embodiments described herein, the composition may comprise a senolytic agent. In some embodiments, the composition comprises quercetin. In some embodiments, the composition comprises dasatinib. In some embodiments, the composition comprises artemisinin. In some embodiments, the composition comprises fisetin. In some embodiments, the composition comprises quercetin and AKG. In some embodiments, the composition comprises quercetin and CaAKG. In some embodiments, the composition comprises dasatinib and AKG. In some embodiments, the composition comprises dasatinib and CaAKG. In some embodiments, the composition comprises dasatinib and quercetin. In some embodiments, the composition comprises dasatinib, quercetin, and AKG. In some embodiments, the composition comprises dasatinib, quercetin, and CaAKG. In some embodiments, the composition comprises artemisinin and AKG. In some embodiments, the composition comprises artemisinin and CaAKG. In some embodiments, the composition comprises fisetin and AKG. In some embodiments, the composition comprises fisetin and CaAKG.

Dosage and Routes of Administration

In some embodiments, the amount of calcium alpha-ketoglutarate is from 50 mg to 5000 mg. In some embodiments, the amount of calcium alpha-ketoglutarate is from 100 mg to 2000 mg. In some embodiments, the therapeutically effective amount of Ca-AKG is at least 350 mg and no greater than 2000 mg. In some instances, the therapeutically effective amount of Ca-AKG is about 350 mg to about 2000 mg. In some instances, the therapeutically effective amount of Ca-AKG is about 400 mg to about 2000 mg. In some instances, the therapeutically effective amount of Ca-AKG is about 450 mg to about 2000 mg. In some instances, the therapeutically effective amount of Ca-AKG is about 500 mg to about 2000 mg. In some instances, the therapeutically effective amount of Ca-AKG is about 550 mg to about 2000 mg. In some instances, the therapeutically effective amount of Ca-AKG is about 600 mg to about 2000 mg. In some instances, the therapeutically effective amount of Ca-AKG is about 650 mg to about 2000 mg. In some instances, the therapeutically effective amount of Ca-AKG is about 700 mg to about 2000 mg. In some instances, the therapeutically effective amount of Ca-AKG is about 800 mg to about 2000 mg. In some instances, the therapeutically effective amount of Ca-AKG is about 900 mg to about 2000 mg. In some instances, the therapeutically effective amount of Ca-AKG is about 1000 mg to about 2000 mg. In some instances, the therapeutically effective amount of Ca-AKG is about 1100 mg to about 2000 mg. In some instances, the therapeutically effective amount of Ca-AKG is about 1200 mg to about 2000 mg. In some instances, the therapeutically effective amount of Ca-AKG is about 1300 mg to about 2000 mg. In some instances, the therapeutically effective amount of Ca-AKG is about 1400 mg to about 2000 mg. In some instances, the therapeutically effective amount of Ca-AKG is about 1500 mg to about 2000 mg. In some instances, the therapeutically effective amount of Ca-AKG is about 1600 mg to about 2000 mg. In some instances, the therapeutically effective amount of Ca-AKG is about 1700 mg to about 2000 mg. In some instances, the therapeutically effective amount of Ca-AKG is about 1800 mg to about 2000 mg. In some instances, the therapeutically effective amount of Ca-AKG is about 1900 mg to about 2000 mg. In some instances, the therapeutically effective amount of Ca-AKG is about 350 mg to about 1900 mg. In some instances, the therapeutically effective amount of Ca-AKG is about 350 mg to about 1800 mg. In some instances, the therapeutically effective amount of Ca-AKG is 350 mg to about 1700 mg. In some instances, the therapeutically effective amount of Ca-AKG is about 350 mg to about 1600 mg. In some instances, the therapeutically effective amount of Ca-AKG is about 350 mg to about 1500 mg. In some instances, the therapeutically effective amount of Ca-AKG is about 350 mg to about 1400 mg. In some instances, the therapeutically effective amount of Ca-AKG is about 350 mg to about 1300 mg. In some instances, the therapeutically effective amount of Ca-AKG is about 350 mg to about 1200 mg. In some instances, the therapeutically effective amount of Ca-AKG is about 350 mg to about 1100 mg. In some instances, the therapeutically effective amount of Ca-AKG is about 350 mg to about 1000 mg. In some instances, the therapeutically effective amount of Ca-AKG is about 350 mg to about 900 mg. In some instances, the therapeutically effective amount of Ca-AKG is about 350 mg to about 800 mg. In some instances, the therapeutically effective amount of Ca-AKG is about 350 mg to about 700 mg. In some instances, the therapeutically effective amount of Ca-AKG is bout 350 mg to about 650 mg. In some instances, the therapeutically effective amount of Ca-AKG is about 350 mg to about 600 mg. In some instances, the therapeutically effective amount of Ca-AKG is about 350 mg to about 550 mg. In some instances, the therapeutically effective amount of Ca-AKG is about 350 mg to about 500 mg. In some instances, the therapeutically effective amount of Ca-AKG is about 350 mg to about 450 mg. In some instances, the therapeutically effective amount of Ca-AKG is about 350 mg to about 400 mg. In some instances, the therapeutically effective amount of Ca-AKG is at least about 350 mg. In some instances, the therapeutically effective amount of Ca-AKG is at least about 400 mg. In some instances, the therapeutically effective amount of Ca-AKG is at least about 450 mg. In some instances, the therapeutically effective amount of Ca-AKG is at least about 500 mg. In some instances, the therapeutically effective amount of Ca-AKG is at least about 550 mg. In some instances, the therapeutically effective amount of Ca-AKG is at least about 600 mg. In some instances, the therapeutically effective amount of Ca-AKG is at least about 650 mg. In some instances, the therapeutically effective amount of Ca-AKG is at least about 700 mg. In some instances, the therapeutically effective amount of Ca-AKG is at least about 750 mg. In some instances, the therapeutically effective amount of Ca-AKG is at least about 800 mg. In some instances, the therapeutically effective amount of Ca-AKG is at least about 900 mg. In some instances, the therapeutically effective amount of Ca-AKG is at least about 1000 mg. In some instances, the therapeutically effective amount of Ca-AKG is at least about 1100 mg. In some instances, the therapeutically effective amount of Ca-AKG is at least about 1200 mg. In some instances, the therapeutically effective amount of Ca-AKG is at least about 1300 mg. In some instances, the therapeutically effective amount of Ca-AKG is at least about 1400 mg. In some instances, the therapeutically effective amount of Ca-AKG is at least about 1500 mg. In some instances, the therapeutically effective amount of Ca-AKG is at least about 1600 mg. In some instances, the therapeutically effective amount of Ca-AKG is at least about 1700 mg. In some instances, the therapeutically effective amount of Ca-AKG is at least about 1800 mg. In some instances, the therapeutically effective amount of Ca-AKG is at least about 1900 mg. In some instances, the therapeutically effective amount of Ca-AKG is at least about 2000 mg.

It is to be understood that in the foregoing embodiments and instances that Ca-AKG may be substituted with the same therapeutically effective amount of a hydrate Ca-AKG. Illustratively, in some embodiments, the amount of a hydrate Ca-AKG is from 50 mg to 5000 mg, from 100 mg to 2000 mg, or other dose range or specific dose described herein. In some embodiments, the therapeutically effective amount is a mono-hydrate Ca-AKG. In some embodiments, the therapeutically effective amount is a hemi-hydrate Ca-AKG. In some embodiments, the therapeutically effective amount is anhydrous Ca-AKG.

In some instances, the therapeutically effective amount of Ca-AKG is at least about 5 mg/kg. In some instances, the therapeutically effective amount of Ca-AKG is at least about 10 mg/kg. In some instances, the therapeutically effective amount of Ca-AKG is at least about 15 mg/kg. In some instances, the therapeutically effective amount of Ca-AKG is at least about 16 mg/kg. In some instances, the therapeutically effective amount of Ca-AKG is at least about 16.1 mg/kg. In some instances, the therapeutically effective amount of Ca-AKG is at least about 16.2 mg/kg. In some instances, the therapeutically effective amount of Ca-AKG is at least about 16.3 mg/kg. In some instances, the therapeutically effective amount of Ca-AKG is at least about 16.4bmg/kg. In some instances, the therapeutically effective amount of Ca-AKG is at least about 16.5 mg/kg. In some instances, the therapeutically effective amount of Ca-AKG is at least about 16.6 mg/kg. In some instances, the therapeutically effective amount of Ca-AKG is at least about 16.7 mg/kg. In some instances, the therapeutically effective amount of Ca-AKG is at least about 16.8 mg/kg. In some instances, the therapeutically effective amount of Ca-AKG is at least about 16.9 mg/kg. In some instances, the therapeutically effective amount of Ca-AKG is at least about 17 mg/kg. In some instances, the therapeutically effective amount of Ca-AKG is at least about 18 mg/kg. In some instances, the therapeutically effective amount of Ca-AKG is at least about 19 mg/kg. In some instances, the therapeutically effective amount of Ca-AKG is at least about 20 mg/kg. In some instances, the therapeutically effective amount of Ca-AKG is at least about 21 mg/kg. In some instances, the therapeutically effective amount of Ca-AKG is at least about 22 mg/kg. In some instances, the therapeutically effective amount of Ca-AKG is at least about 23 mg/kg. In some instances, the therapeutically effective amount of Ca-AKG is at least about 24 mg/kg. In some instances, the therapeutically effective amount of Ca-AKG is at least about 25 mg/kg. In some instances, the therapeutically effective amount of Ca-AKG is at least about 28 mg/kg. In some instances, the therapeutically effective amount of Ca-AKG is at least about 30 mg/kg. In some instances, the therapeutically effective amount of Ca-AKG is at least about 33 mg/kg. In some instances, the therapeutically effective amount of Ca-AKG is at least about 35 mg/kg. In some embodiments, the therapeutically effective amount of Ca-AKG is at least 16.6 mg/kg.

In some embodiments, the compositions may further comprise vitamin A. In some instances, the composition comprises at least about 100 mcg of vitamin A. In some instances, the composition comprises at least about 150 mcg of vitamin A. In some instances, the composition comprises at least about 200 mcg of vitamin A. In some instances, the composition comprises at least about 250 mcg of vitamin A. In some instances, the composition comprises at least about 300 mcg of vitamin A. In some instances, the composition comprises at least about 350 mcg of vitamin A. In some instances, the composition comprises at least about 400 mcg of vitamin A. In some instances, the composition comprises at least about 410 mcg of vitamin A. In some instances, the composition comprises at least about 420 mcg of vitamin A. In some instances, the composition comprises at least about 430 mcg of vitamin A. In some instances, the composition comprises at least about 440 mcg of vitamin A. In some instances, the composition comprises at least about 450 mcg of vitamin A. In some instances, the composition comprises at least about 460 mcg of vitamin A. In some instances, the composition comprises at least about 470 mcg of vitamin A. In some instances, the composition comprises at least about 480 mcg of vitamin A. In some instances, the composition comprises at least about 490 mcg of vitamin A. In some instances, the composition comprises at least about 500 mcg of vitamin A. In some instances, the composition comprises at least about 550 mcg of vitamin A. In some instances, the composition comprises at least about 600 mcg of vitamin A. In some instances, the composition comprises at least about 650 mcg of vitamin A. In some instances, the composition comprises at least about 700 mcg of vitamin A. In some instances, the composition comprises at least about 750 mcg of vitamin A. In some instances, the composition comprises at least about 800 mcg of vitamin A. In some instances, the composition comprises at least about 850 mcg of vitamin A. In some instances, the composition comprises at least about 900 mcg of vitamin A.

In some embodiments, the compositions may further comprise vitamin D3. In some instances, the composition comprises at least about 8.5 mcg of vitamin D3. In some instances, the composition comprises at least about 9 mcg of vitamin D3. In some instances, the composition comprises at least about 9.5 mcg of vitamin D3. In some instances, the composition comprises at least about 10 mcg of vitamin D3. In some instances, the composition comprises at least about 10.5 mcg of vitamin D3. In some instances, the composition comprises at least about 11 mcg of vitamin D3. In some instances, the composition comprises at least about 11.5 mcg of vitamin D3. In some instances, the composition comprises at least about 12 mcg of vitamin D3. In some instances, the composition comprises at least about 12.1 mcg of vitamin D3. In some instances, the composition comprises at least about 12.2 mcg of vitamin D3. In some instances, the composition comprises at least about 12.3 mcg of vitamin D3. In some instances, the composition comprises at least about 12.4 mcg of vitamin D3. In some instances, the composition comprises at least about 12.5 mcg of vitamin D3. In some instances, the composition comprises at least about 12.6 mcg of vitamin D3. In some instances, the composition comprises at least about 12.7 mcg of vitamin D3. In some instances, the composition comprises at least about 12.8 mcg of vitamin D3. In some instances, the composition comprises at least about 12.9 mcg of vitamin D3. In some instances, the composition comprises at least about 13 mcg of vitamin D3. In some instances, the composition comprises at least about 13.5 mcg of vitamin D3. In some instances, the composition comprises at least about 14 mcg of vitamin D3. In some instances, the composition comprises at least about 14.5 mcg of vitamin D3. In some instances, the composition comprises at least about 15 mcg of vitamin D3. In some instances, the composition comprises at least about 20 mcg of vitamin D3. In some instances, the composition comprises at least about 25 mcg of vitamin D3. In some instances, the composition comprises at least about 300 IU of vitamin D3. In some instances, the composition comprises at least about 350 IU of vitamin D3. In some instances, the composition comprises at least about 400 IU of vitamin D3. In some instances, the composition comprises at least about 450 IU of vitamin D3. In some instances, the composition comprises at least about 460 IU of vitamin D3. In some instances, the composition comprises at least about 470 IU of vitamin D3. In some instances, the composition comprises at least about 480 IU of vitamin D3. In some instances, the composition comprises at least about 490 IU of vitamin D3. In some instances, the composition comprises at least about 500 IU of vitamin D3. In some instances, the composition comprises at least about 510 IU of vitamin D3. In some instances, the composition comprises at least about 520 IU of vitamin D3. In some instances, the composition comprises at least about 530 IU of vitamin D3. In some instances, the composition comprises at least about 540 IU of vitamin D3. In some instances, the composition comprises at least about 550 IU of vitamin D3. In some instances, the composition comprises at least about 600 IU of vitamin D3. In some instances, the composition comprises at least about 650 IU of vitamin D3. In some instances, the composition comprises at least about 700 IU of vitamin D3. In some instances, the composition comprises at least about 750 IU of vitamin D3. In some instances, the composition comprises at least about 800 IU of vitamin D3. In some instances, the composition comprises at least about 850 IU of vitamin D3. In some instances, the composition comprises at least about 900 IU of vitamin D3.

In some embodiments, the composition comprises CaAKG and Vitamin A. In some embodiments, the composition comprising CaAKG and Vitamin A is administered to a male subject. In some embodiments, the composition comprising CaAKG and Vitamin A is administered to a human male subject. In some embodiments, the composition comprises Ca-AKG nd Vitamin D3. In some embodiments, the composition comprising CaAKG and Vitamin D3 is administered to a female subject. In some embodiments, the composition comprising CaAKG and Vitamin D3 is administered to a human female subject.

The compositions according to the disclosure herein may be administered via a variety of routes. In some embodiments, the composition is formulated for oral administration. In some embodiments, the composition is formulated for sublingual administration. In other embodiments, the composition is formulated for injection. In some embodiments, the composition is formulated for topical administration.

In some embodiments, the compounds described herein are formulated in oral dosage forms. Two or more compounds according to the invention are formulated by combining them with, e.g., pharmaceutically acceptable carriers or excipients. In various embodiments the compounds according to the invention are formulated in oral dosage forms including, by way of example only, tablets, powders, granules, pills, dragees, capsules, liquids, serums, gels, solutions, syrups, elixirs, slurries, suspensions, emulsions and the like.

### Orally Administered Forms

In certain embodiments, preparations containing the active agents for oral use are obtained by mixing one or more solid excipient with one or more of the compounds described herein, optionally grinding the resulting mixture, and processing the mixture of granules, after adding suitable auxiliaries, if desired, to obtain tablets, pills, or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross linked croscarmellose sodium, polyvinylpyrrolidone, agar, or alginic acid or a salt thereof such as sodium alginate.

In one embodiment, dosage forms, such as dragee cores, pills, and tablets, are provided with one or more suitable coatings. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of the active agents described herein are formulated into other solid oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added. In some embodiments, AKG or Ca-AKG is formulated into a soft gel capsule.

In some embodiments, AKG or CaAKG are formulated as coated beads as described in Patel, RR and Patel JK, “Novel Technologies of Oral Controlled Release Drug Delivery System,” Systematic Reviews in Pharmacy, July-December 2010, Vol. 1 (2), 128-132.

In some embodiments, AKG is formulated with fish oil in a gel cap. In some embodiments, AKG or CaAKG is formulated into an amino acid supplement including one or more of the following amino acids, L-histidine, L-isoleucine, L-leucine, L-lysine, L-methionine, L-phenylalanine, L-threonine, L-valine. In some embodiments, AKG or CaAKG is added as a source of glutamic acid.

In other embodiments, active agents described herein are formulated into oral liquid dosage forms. Exemplary liquid preparations for oral use include solutions, emulsions, serums, solutions, syrups or suspensions containing one or more active ingredients in a suitable vehicle. Syrups are clear viscous oral liquids containing high concentrations of sugar or other sweetening agents, in which active agents are solubilized in a pharmaceutically acceptable vehicle. Suspensions consist of finely divided particles of active agent suspended in pharmaceutically acceptable vehicle in which the particles are poorly soluble. Oral emulsions contain liquid forms of active agents dispersed as droplets in a continuous phase of another immiscible vehicle with the help of emulsifying agents (e.g. carbohydrates, gelatin, high molecular weight alcohols, wetting agents, colloidal clays, and the like).

In some embodiments, active agents are formulated into semi-solid oral dosage forms such as gels. Gels or jelly-like formulations have particular relevance for elderly or dysphagic patients with difficulty consuming other oral dosage forms. Gels are formed by adding active agents to water, adding a low critical concentration (e.g. 0.5-2.5%) of a gelling agent, heating, and cooling. Examples of suitable gelling agents include agar, gelatin, carrageenan, sodium caseinate, glycerogelatin, silk fibroin, gellan gum, kelcogel, xyloglucan, gellan, and pectin.

In some embodiments, the one or more active agents are formulated into a food product. In some embodiments, the food product is a drink for oral administration. Non-limiting examples of a suitable drink include fruit juice, a fruit drink, an artificially flavored drink, an artificially sweetened drink, a carbonated beverage, a sports drink, a liquid dairy product, a shake, a smoothie, or a caffeinated beverage. In some embodiments, the one or more active agents are a powder for constitution in the drink for oral administration.

In some embodiments, the food product is a solid foodstuff for oral administration. Suitable examples of a solid foodstuff include without limitation a food bar, a snack bar, a jelly bean, sports gel, a gummy, a sports chew, a cookie, a brownie, a muffin, a cracker, an ice cream bar, or a frozen yogurt bar.

In some embodiments, the food product is a sports drink. In some embodiments, the sports drink contains the active agents and sports drinks ingredients, including but not limited to, filtered water, brominated vegetable oil, high fructose corn syrup, citric acid, fruit juice, salt, sodium citrate, monopotassium phosphate, glycerol ester of wood rosin, and artificial colors.

In some embodiments, the food product is a smoothie. In some embodiments, the smoothie contains the active agents and smoothie ingredients, including but not limited to, milk (cow's milk, soy milk, or almond milk), yogurt, nut butter (peanut, almond or sun butter), honey, and ice. In some embodiments, the smoothie contains frozen mixed berries.

In some embodiments, the food product is a food bar. In some embodiments, the food bar contains the active agents and food bar ingredients, including but not limited to brown rice syrup, rolled oats, soy protein isolate, cane syrup, roasted soybeans, rice flour, dried cane syrup, unsweetened chocolate, soy flour, oat fiber, high oleic sunflower oil, cocoa butter, barley malt extract, sea salt, natural flavors, soy lecithin, and cinnamon.

In some embodiments, the food product is a jelly bean. In some embodiments, the jelly bean contains the active agents and jelly bean ingredients, including but not limited to cane sugar, tapioca syrup, citric acid, apple juice from concentrate, lime juice from concentrate, raspberry juice from puree, pear juice from concentrate, natural flavor, thiamine hydrochloride, riboflavin, niacinamide, ascorbic acid, potassium citrate, sodium citrate, sodium lactate, confectioners glaze, vegetable and fruit juice for coloring, curcumin, beeswax, carnauba wax, and salt.

In some embodiments, the food product is a sports gel. In some embodiments, the sports gel contains the active agents and sports gel ingredients, including but not limited to maltodextrin, water, fructose, leucine, sea salt, citric acid, natural flavor, potassium citrate, sodium citrate, calcium carbonate, valine, green tea (leaf) extract, gellan gum, isoleucine, sunflower oil, sodium benzoate, and potassium sorbate.

In some embodiments, the food product is a gummy. In some embodiments, the gummy contains the active agents and gummy ingredients, including but not limited to cane sugar, glucose, water, citrus pectin, citric acid, trisodium citrate, natural flavor (strawberry), and natural color (black carrot concentrate).

In some embodiments, the food product is a sports chew. In some embodiments, the sports chew contains the active agents and sports chew ingredients, including but not limited to brown rice syrup, evaporated cane juice, brown rice syrup solids, pectin, citric acid, colored with black carrot juice concentrate, natural flavor, sunflower oil, and carnauba wax.

### Animal Feed

In certain embodiments, the active agents are included in a diet which can comprise any suitable pet food formulation which also provides adequate nutrition for a non-human animal. For example, a typical canine diet for use in the present invention may contain about 18-40% crude protein, about 4-30% fat, and about 4-20% total dietary fiber. However, no specific ratios or percentages of these or other nutrients are required. Examples of detailed preparation of animal feed from base ingredients are found elsewhere, for e.g. in U.S. Pat. No. 4,045,585, US20100303968, and U.S. Pat. No. 3,875,304. In some embodiments, the therapeutically effective amount of Ca-AKG in animal feed is at least about 0.025% w/w to at least about 10% w/w. In some embodiments, the therapeutically effective amount of Ca-AKG in animal feed is at least about 0.025% w/w. In some embodiments, the therapeutically effective amount of Ca-AKG in animal feed is at least about 0.05% w/w. In some embodiments, the therapeutically effective amount of Ca-AKG in animal feed is at least about 0.075% w/w. In some embodiments, the therapeutically effective amount of Ca-AKG in animal feed is at least about 0.1% w/w. In some embodiments, the therapeutically effective amount of Ca-AKG in animal feed is at least about 0.3% w/w. In some embodiments, the therapeutically effective amount of Ca-AKG in animal feed is at least about 0.5% w/w. In some embodiments, the therapeutically effective amount of Ca-AKG in animal feed is at least about 0.8% w/w. In some embodiments, the therapeutically effective amount of Ca-AKG in animal feed is at least about 1% w/w. In some embodiments, the therapeutically effective amount of Ca-AKG in animal feed is at least about 2% w/w. In some embodiments, the therapeutically effective amount of Ca-AKG in animal feed is at least about 3% w/w. In some embodiments, the therapeutically effective amount of Ca-AKG in animal feed is at least about 4% w/w. In some embodiments, the therapeutically effective amount of Ca-AKG in animal feed is at least about 5% w/w. In some embodiments, the therapeutically effective amount of Ca-AKG in animal feed is at least about 6% w/w. In some embodiments, the therapeutically effective amount of Ca-AKG in animal feed is at least about 7% w/w. In some embodiments, the therapeutically effective amount of Ca-AKG in animal feed is at least about 8% w/w. In some embodiments, the therapeutically effective amount of Ca-AKG in animal feed is at least about 9% w/w. In some embodiments, the therapeutically effective amount of Ca-AKG in animal feed is at least about 10% w/w.

### Altered Release

A composition comprising any of the active agents described herein may be formulated for sustained or slow release, also called timed release or controlled release. Such compositions may generally be administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of agent contained within a sustained release formulation depends upon the site of implantation, the rate and expected duration of release, and the nature of the condition, disease or disorder to be treated or prevented.

In some embodiments, AKG or Ca-AKG formulated as a sustained release tablet.

### Injectable

In still other embodiments, the active agents described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, formulations for parenteral administration include aqueous solutions of the active compounds in water-soluble form. In additional embodiments, suspensions of the active agents are prepared as appropriate oily injection suspensions. Suitable lipophilic solvents or vehicles for use in the compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

### Topical

In still other embodiments, the active agents are administered topically. The compounds described herein are formulated into a variety of topically administrable compositions, such as solutions, suspensions, lotions, gels, foams, serums, pastes, medicated sticks, balms, creams or ointments. Such compositions optionally contain solubilizers, stabilizers, tonicity enhancing agents, buffers and preservatives.

Compositions disclosed herein can be formulated as emulsions for topical application. An emulsion contains one liquid distributed in the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

In certain embodiments, the compositions comprising the active agents are formulated for topical administration using a bandage or transdermal patch, or as a powder/talc or other solid, liquid, spray, aerosol, ointment, foam, cream, gel, or paste. This preferably is in the form of a controlled release formulation or sustained release formulation administered topically or injected directly into the skin adjacent to or within the area to be treated, e.g., intradermally or subcutaneously. The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

### Hair Care Products

In some embodiments, the active agents according to the invention are formulated into a hair care product. In some embodiments, the active agent in the hair care product comprises alpha-ketoglutarate salt. Examples of hair care products useful for administering the two or more compounds include a shampoo, a conditioner, a hair spray, or a moisturizer. In some embodiments, the alpha-ketoglutarate is formulated in a shampoo. A shampoo is a preparation comprising a surfactant (for e.g. sodium lauryl sulfate) and other additives specifically selected to remove surface grease, dirt, and skin debris from the hair shaft and scalp. An exemplary liquid shampoo formulation would be an aqueous solution containing 40% sodium lauryl sulfate, 2-4% sodium chloride (adjusted to desired viscosity), an effective amount of the 2 or more compounds, a preservative, and optional perfumes or colors. A conditioner or moisturizer is a preparation comprising a conditioning or moisturizing substance which adheres to hair in the presence of water. Examples of conditioning or moisturizing substances suitable for use in conditioners include quaternized surfactants, cationic polymers, silicone compounds (for e.g. polydimethylsiloxane, cyclomethicone), emollients, and humectants. An exemplary hair spray comprises a near 50:50 mix of buffered water and ethanol as diluents, alongside low concentrations of a humectant (e.g. glycerol), a conditioner, and a hair styling polymer (e.g. PVP K-30).

In some embodiments, AKG is formulated into a hair care product for men. In some embodiments, the AKG hair care product for men is for hair re-growth. In some embodiments, the AKG is an ester of AKG. In some embodiments, the ester of AKG is a methyl ester of AKG. In some embodiments, the ester of AKG is a dimethyl ester of AKG. In some embodiments, the ester is an ethyl ester of AKG. In some embodiments, the ester is a diethyl ester of AKG. In some embodiments, the AKG hair care product for men further comprises a vitamin A compound. In some embodiments, the vitamin A compound is selected from the group consisting of retinol ester, retinol, retinal, retinoic acid, and retinoic acid salts. In some embodiments, the AKG hair care product for men further comprises a vitamin E compound. In some embodiments, the vitamin E compound is alpha-tocopherol. In some embodiments, the AKG hair care product for men further comprises dihydroquercetin-glucoside. In some embodiments, the AKG hair care product for men further comprises one or more excipients.

In some embodiments, the AKG hair care product for men is formulated as a foam. In some embodiments, the foam is formulated with minoxidil. In some embodiments, minoxidil is formulated into the foam at 5% w/w. In some embodiments, the foam contains additional ingredients, including, but not limited to butane, butylated hydroxytoluene, cetyl alcohol, citric acid, glycerin, isobutene, lactic acid, polysorbate 60, propane, purified water, SD alcohol 40-B, stearyl alcohol.

In some embodiments, the AKG hair care product for men is formulated as a shampoo. In some embodiments, the shampoo comprises minoxidil. In some embodiments, the shampoo comprises biotin. In some embodiments, the shampoo comprises ketoconazole. In some embodiments, the shampoo comprises argan oil. In some embodiments, the argan oil comprises tocopherols, phenols, carotenes, squalene, and fatty acids. In some embodiments, the fatty acids are 80% unsaturated fatty acids. In some embodiments, the phenols comprise caffeic acid, oleuropein, vanillic acid, tyrosol, catechol, resorcinol, (−)-epicatechin, and (+)-catechin.

In some embodiments, the AKG hair care product for men is used in combination with a formulation comprising matrikine, apigenin, and oleanolic acid.

In some embodiments, the AKG hair care product for men is used in combination with a formulation comprising butylene glycol, water, dextran, acetyl tetrapeptide-3, and trifolium pratense (clover) flower extract.

In some embodiments, the AKG hair care product for men is used in combination with a formulation comprising hydrolyzed vegetable protein pg-propyl silanetriol and water. In some embodiments, the AKG hair care product for men is used in combination with a formulation comprising dihydroquercetin-glucoside, epigallaocatechin gallate-glucoside, glycine, zinc chloride, meta-bisulfate, glycerin, and water. In some embodiments, the dihydroquercetin-glucoside is formulated at approximately 0.005%. In some embodiments, the epigallaocatechin gallate-glucoside is formulated at approximately 0.0009%. In some embodiments, the glycine is formulated at approximately 0.005%. In some embodiments, the zinc chloride is formulated at approximately 0.002%. In some embodiments, the meta-bisulfite is formulated at approximately 0.015%. In some embodiments, the glycerin is formulated at approximately 50%.

In some embodiments, the AKG hair care product for men is used in combination with a hair regrowth tablet. In some embodiments, the hair regrowth table comprises aminomar marine complex, vitamin C, zinc, and horsetail stem extract.

In some embodiments, AKG and vitamin D are formulated into a hair care product for women. In some embodiments, the AKG and vitamin D hair care product for women is for hair re-growth. In some embodiments, the vitamin D comprises vitamin D₂. In some embodiments, the vitamin D comprises vitamin D3. In some embodiments, the vitamin D comprises vitamin D4. In some embodiments, the vitamin D comprises vitamin D₅. In some embodiments, the vitamin D comprises vitamin D₁. In some embodiments, the AKG and vitamin D hair care product for women further comprises a vitamin A compound. In some embodiments, the vitamin A compound is selected from the group consisting of retinol ester, retinol, retinal, retinoic acid, and retinoic acid salts. In some embodiments, the AKG and vitamin D hair care product for women further comprises a vitamin E compound. In some embodiments, the vitamin E compound is alpha-tocopherol. In some embodiments, the AKG and vitamin D hair care product for women further comprises dihydroquercetin-glucoside. In some embodiments, the AKG and vitamin D hair care product for women further comprises one or more excipients.

In some embodiments, the AKG and vitamin D hair care product for women is formulated as a foam. In some embodiments, the foam is formulated with minoxidil. In some embodiments, minoxidil is formulated into the foam at 5% w/w. In some embodiments, the foam contains additional ingredients, including, but not limited to butane, butylated hydroxytoluene, cetyl alcohol, citric acid, glycerin, isobutene, lactic acid, polysorbate 60, propane, purified water, SD alcohol 40-B, stearyl alcohol.

In some embodiments, the AKG and vitamin D hair care product for women is formulated as a shampoo. In some embodiments, the shampoo comprises minoxidil. In some embodiments, the shampoo comprises biotin. In some embodiments, the shampoo comprises ketoconazole. In some embodiments, the shampoo comprises argan oil. In some embodiments, the argan oil comprises tocopherols, phenols, carotenes, squalene, and fatty acids. In some embodiments, the fatty acids are 80% unsaturated fatty acids. In some embodiments, the phenols comprise caffeic acid, oleuropein, vanillic acid, tyrosol, catechol, resorcinol, (−)-epicatechin, and (+)-catechin.

In some embodiments, the AKG and vitamin D hair care product for women is used in combination with a formulation comprising matrikine, apigenin, and oleanolic acid.

In some embodiments, the AKG and vitamin D hair care product for women is used in combination with a formulation comprising butylene glycol, water, dextran, acetyl tetrapeptide-3, and trifolium pratense (clover) flower extract.

In some embodiments, the AKG and vitamin D hair care product for women is used in combination with a formulation comprising hydrolyzed vegetable protein pg-propyl silanetriol and water.

In some embodiments, the AKG and vitamin D hair care product for women is used in combination with a formulation comprising dihydroquercetin-glucoside, epigallaocatechin gallate-glucoside, glycine, zinc chloride, meta-bisulfate, glycerin, and water. In some embodiments, the dihydroquercetin-glucoside is formulated at approximately 0.005%. In some embodiments, the epigallaocatechin gallate-glucoside is formulated at approximately 0.0009%. In some embodiments, the glycine is formulated at approximately 0.005%. In some embodiments, the zinc chloride is formulated at approximately 0.002%. In some embodiments, the meta-bisulfate is formulated at approximately 0.015%. In some embodiments, the glycerin is formulated at approximately 50%. In some embodiments, the AKG and vitamin D hair care product for women is used in combination with a hair regrowth tablet. In some embodiments, the hair regrowth table comprises aminomar marine complex, vitamin C, biotin, and iron.

### Administration Protocol and Schedule

In some embodiments one or more of the active agents are administered separately. In some embodiments, the active agents administered separately are administered in separate dosage units (e.g. pills, dragees, tablets). In some embodiments, the active agents administered separately are administered via separate routes of administration. In certain embodiments, two or more of the active agents are administered separately. In certain embodiments, three of the active agents are administered separately. In certain embodiments, the active agents administered separately are not administered simultaneously.

In certain embodiments, the active agents not administered simultaneously are administered within a defined window. In specific embodiments, the defined window is 48, 36, 24, 12, or 6 hours. In some embodiments one or more, two or more, or three of the active agents are administered within the defined window.

In some embodiments, the active agents or compositions thereof used for treatment are administered for a particular treatment period. In some embodiments, the active agents or compositions thereof are administered for a chronic treatment period, that is, for an extended period of time, including throughout the duration of the patient's life in order to ameliorate or otherwise control or limit the symptoms of the patient's disease or condition. Within the treatment period, the active agents or compositions thereof used for treatment are administered on a particular time schedule.

In further embodiments, the active agents or compositions thereof are administered one, two, three, or four times daily. In some embodiments, the composition is administered once daily. In some embodiments, the composition is administered twice daily. In some embodiments, the composition is administered three times daily. In some embodiments, the composition is administered four times daily. In some embodiments, the active agents or compositions thereof are administered in the morning and evening. In some embodiments, the active agents or compositions thereof are administered one, two, three, or four times weekly. In some embodiments, the active agents or compositions thereof are administered one, two, three, or four times monthly.

In some embodiments, the active agents or compositions thereof are administered for at least two months. In some embodiments, the active agents or compositions thereof are administered for at least three months. In some embodiments, the active agents or compositions thereof are administered for at least four months. In some embodiments, the active agents or compositions thereof are administered for at least five months. In some embodiments, the active agents or compositions thereof are administered for at least six months. In some embodiments, the active agents or compositions thereof are administered for at least seven months. In some embodiments, the active agents or compositions thereof are administered for at least eight months. In some embodiments, the active agents or compositions thereof are administered for at least nine months. In some embodiments, the active agents or compositions thereof are administered for at least ten months. In some embodiments, the active agents or compositions thereof are administered for at least twelve months. In some embodiments, the active agents or compositions thereof are administered for at least fourteen months. In some embodiments, the active agents or compositions thereof are administered for at least sixteen months. In some embodiments, the active agents or compositions thereof are administered for at least eighteen months. In some embodiments the active agents or compositions are taken for at least three months with one year. In some embodiments, the active agents or compositions are taken one month of every six months.

### EXAMPLES

### Example 1: Effect of AKG on Frailty, Lifespan and Healthspan

### Animal Housing and Diet

All mice were housed on a 12-h light/dark cycle and kept at 20-22° C. Mice were maintained on a regular mouse diet (Teklad Irradiated 18% protein diet-2918) until they reached 18th month of age when treatment started. AKG treated animals were subjected to a lifelong 2% (w/w) AKG supplement on 2918 diet while Control group were kept on standard-2918 diet. Pure calcium 2-oxoglutarate was homogeneously mixed during manufacturing of the 2918 diet. The principle endpoint of the health span and lifespan study was natural death. Two independent cohorts of C57BL6/J mice were around 14th months of age. All animals were housed on a 12-h light/dark cycle and in a pathogen-free facility at 20-22° C. Mice were aged on a regular mouse diet (Teklad Irradiated Global 18% Protein-2918) until they were ready for the experiment. Mice were housed in groups (5 per cage at a maximum) and aggressive male mice were isolated to prevent fighting. All lifespan and healthspan experiments were started around 18th month of age. Animals were initially based lined and grouped afterwards. AKG treatment group received Calcium alpha-ketoglutarate supplemented diet (2% w/w, 2918). Pure Calcium alpha-ketoglutarate was homogeneously mixed during manufacturing of the 2918 diet prior to irradiation and pelleting. Mice were inspected daily, and medicated for non-life threatening conditions as directed by the veterinary staff

Test mice are fed AKG(2% (w/w) or Standard diet at 18 months of age. The study comprises two cohorts (n=182 animal total) and a sacrifice group (n=12). Cohort-1 mice are used for frailty index measurements and lifespan. Cohort-2 mice are started on diets at the same age for replication of survival, metabolic studies and complementary aging studies. Mice are observed until natural death.

| | | |
|---|---|---|
| Cohort-1 | n = 90 animals | Measuring 31 |
| | (n = 44, female and n = 46, male) | Clinically relevant |
| | Age at start of the treatment: | frailty index (FI) |
| | 18^{th} Months old | Lifespan |
| | Treatment Duration: Life long | |
| Cohort-2 | n = 93 animals | Lifespan |
| | (n = 45, female and n = 48, male) | Weight and |
| | Age at start of the treatment: | body compositions |
| | 18^{th} Months old | Metabolic cages |
| | Treatment Duration: Life long | Heart Function |
| Sacrificed | n = 12 (female) | Blood and |
| group | Age at start of the treatment: | Tissue collection |
| | 17^{th} Months old | |
| | Treatment Duration: 3 months | |

### Survival

The principle endpoint of health span and lifespan study was natural death or mice found dead at daily inspection or age at euthanasia (for mice deemed unlikely to survive for next 48 hr and are in enormous discomfort). The criteria for euthanasia were based on an independent assessment by a veterinarian, according to AAALAC guidelines and only where the condition of the animal was considered. Severe lethargy, rapid weight loss (over two weeks>20%), severe distended abdomen and body condition score with signs of pain (grimace), inability to move despite the stimuli, severe ulcer or bleeding tumor, sever temperature loss with abnormal breathing rate. Animal found dead or euthanized was necropsied for pathology score. No invasive measurements were performed on this population, n=180 animals (two cohorts of 90 animals). A sacrificed group were purchased at 14 months of age, baselined and grouped the following week. The mice, n=12 were either receiving Teklad-2918 or 2% w/w AKG supplemented 2918. Animals were sacrificed and tissues were collected after 3 months of treatment. Food intake and body weight were measured on a biweekly and bimonthly basis for the duration of the study.

### Aging Index (Frailty Index)

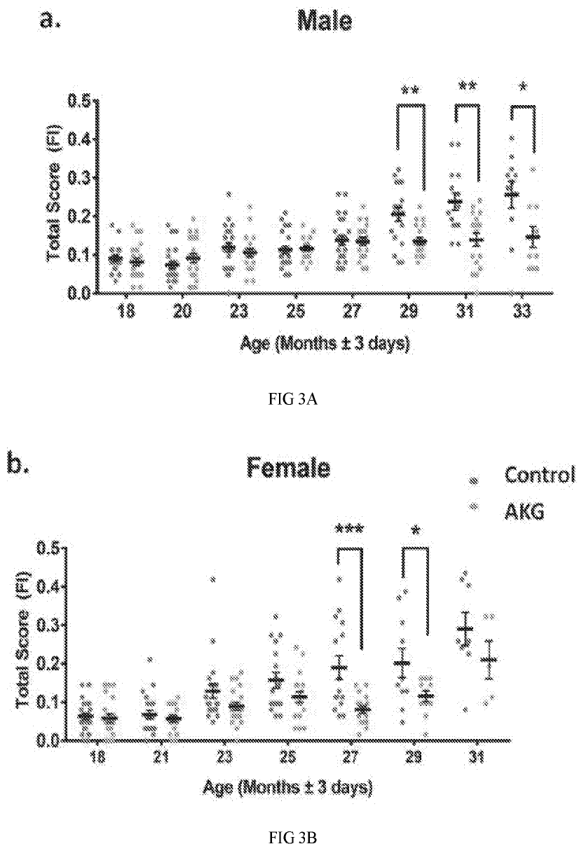

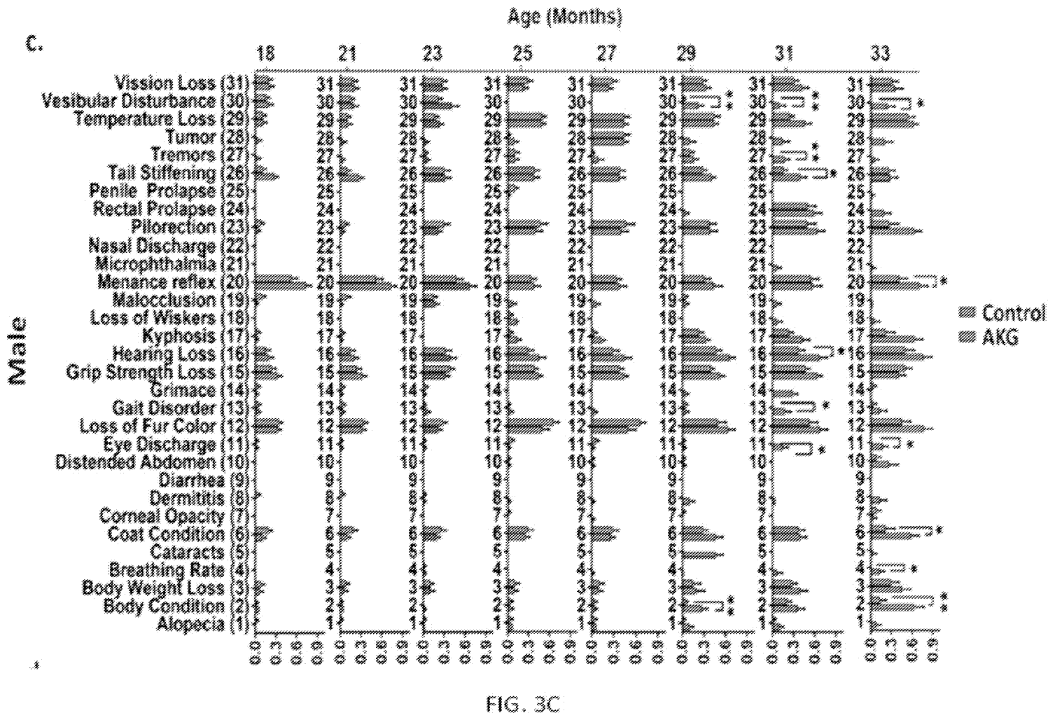

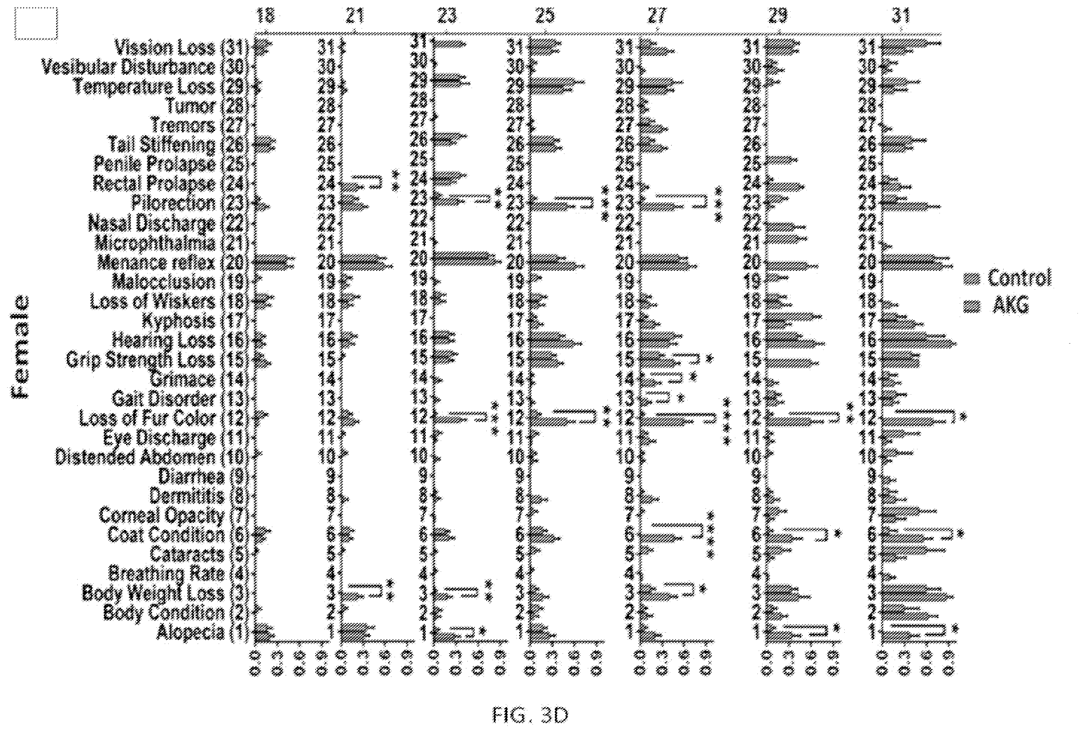

For the subjective properties of the assessments, all measurements were completely blinded. These assessments indicate age-associated deterioration of health and include evaluation of the animal musculoskeletal system, the vestibulocochlear/auditory systems, the ocular and nasal systems, the digestive system, the urogenital system, the respiratory system, signs of discomfort, body weight and body surface temperature. 0 is assigned if no sign of frailty is observed and the animal is healthy for that phenotype. A moderate phenotype and a severe phenotype is scored 0.5 and 1 respectively. Loss of temperature and weight were scored using standard deviation for the study (Table 2). All the phenotypes were scored as described before by Whitehead, J. C., et al., except for temperature and weight. Temperature and weights scoring: Briefly, for weight and temperature, the new scaling scores were used; Average and standard deviations (STDEV) were calculated sex specifically using our own baseline data sets (data collected before the start of the treatment mice at 18th month age). A decrease in temperature or weight (FIG. 3B, and FIG. 3D) within one STDEV is scored as a (0), a decrease bigger than one STDEV but smaller than 2 STDEV will be scored as a (0.5) and any decrease more than 2 STDEV is scored as a (1).

TABLE 2
List of assessed deficits in aged-mice-Frailty index.

| | | |
|---|---|---|
| Integument: | Vestibulocochlear/Auditory: | Respiratory system: |
| 1. Alopecia | 14. Vestibular disturbance | Breathing rate/depth |
| 2. Loss of fur color | 15. Hearing loss | |
| 3. Dermatitis | 16. Cataracts | |
| 4. Loss of whiskers | 17. Eye discharge/swelling | |
| 5. Coat condition | 18. Microphthalmia | |
| | 19. Corneal opacity | |
| | 20. Vision loss | |
| | 21. Menace reflex | |
| | 22. Nasal discharge | |
| Physical/Musculoskeletal: | Digestive/Urogenital: | Discomfort and others: |
| 6. Tumors | 23. Malocclusions | 28. Mouse Grimace |
| 7. Distended abdomen | 24. Rectal prolapse | 29. Piloerection |
| 8. Kyphosis | 25. Vaginal/uterine/Penile prolapse | 30. temperature* |
| 9. Tail stiffening | 26. Diarrhea | 31. Weight* |
| 10. Gait disorders | | |
| 11. Tremor | | |
| 12. Forelimb grip strength | | |
| 13. Body condition score | | |

### Statistical Analyses

Python Software was used to extract all the health span data and create files compatible with R software for analysis. Data were analyzed using R, GraphPad Prism 7 and OASIS 2 software. Log-rank (Mantel-Cox) tests were used to analyze Kaplan-Meier curves, and a fisher's exact test was performed for maximum lifespan analysis (at 90% survival). Two-tailed Student's t-tests were used for analyses of scoring at each time point between control and AKG treated group. Two-way analysis of variance (ANOVA) with Bonferroni post hoc correction was used for analysis between morbidity curves. The area under curve (AUC) of mortality graphs were measured baselining at 18 months of age. The changes in AUC were used to calculate the percent compression of morbidity.

### Inflammatory Cytokines and Chemokines

Blood samples were collected from the jugular vein of young (18 months old), aged control and AKG fed (29 months old) animals. The samples were measured for soluble cytokines and chemokines in serum using multiplex bead array technology (MD31).

### Metabolic Data

Metabolism was measured applying indirect calorimetry. Promethion metabolic cage system-Sable Systems International was used. The system is equipped with GA-3 small mammal gas analyzers for measurements of O₂ (consumption) and CO₂ (production). Energy expenditure, food intake, water consumption, body weight, physical activity and volunteer exercise were simultaneously recorded over 4 consecutive days (96 hours). Mice were housed individually in metabolic cages and accustomed to their environment a day before the start of recording. Data were analyzed using Sable System Expedata-P Data Analysis Software. Subsequently, CalR software, a free web tool was used for analysis of experiments using indirect calorimetry, to analyze the raw data, generate some plots and run statistical analysis. The whole-body composition analysis was conducted using a quantitative nuclear magnetic resonance machine (EchoMRI-2012, Echo Medical Systems).

### Transthoracic Echocardiography

Transthoracic echocardiography Echocardiography examination was performed using a high resolution (32-55 MHz) Visualsonics Vevo 2100 micro-ultrasound system with the echocardiography probe (MS-400). Individual mice were placed on a heating pad (37° C.) and minor sedation (0.5% isoflurane oxygen) was used to paralyze the animal (minimizing the cardiac suppression side effect) during the measurement time. Doppler imaging, 2D and M-mode echocardiography was performed to evaluate cardiac morphometry, systolic function, and mean baseline myocardial performance index (MPI).

### Treadmill Exhaustion Test

Since, mice were old (28 months old) the initial speed and the acceleration were adjusted for the study. All mice were trained and adapted to the environment for three consecutive days for 10 min at 5 m/min before the actual experiment. On the day of experiment, mice were warmed up for 3 min at 5 m/min after which the speed was accelerated by 1.5 m/min-2. Air puffs were used as stimuli to keep the animal active. The maximal speed and distance were recorded once the mice were exhausted (signs of exhaustion including heavy breathing, hunched back and unwillingness of the animal to get on the treadmill belt despite 10 air puffs).

### Western

Mice were fasted overnight, the next morning tissues; heart, lung, kidney, adipose tissue (visceral fat in females, carefully along the epididymis and the uterus) and skin (back skin, on the spine midway between the head and the tail) were dissected and immediately frozen in liquid nitrogen. Tissues were homogenized using the Omni TH homogenizer (Omni International) on ice in Radioimmunoprecipitation assay (RIPA) buffer; 300 mM NaCl, 1.0% NP-40, 0.5% sodium deoxycholate, 0.1% SDS, 50 mM Tris (pH 8.0), protease inhibitor cocktail (Roche) and phosphatase inhibitor 2, 3 (Sigma). Samples were centrifuged at 13,200 rpm for 10 min, 4° C. The protein contents of the supernatants were assessed using the detergent compatible (DC) protein assay (Bio-Rad). Equal amounts of protein were resolved by SDS-PAGE (4%-12% Bis-Tris gradient gel, Invitrogen), transferred to nitrocellulose membranes, and incubated with protein/phosphoprotein-specific antibodies. The antibodies against phosphorylated rsS6^{S240/244} (5364), Akt^{S473} (4058), S6(2217), Akt (4691), and glyceraldehyde 3-phoshate dehydrogenase (GAPDH; 2118) were used. Protein bands were revealed using the Amersham enhanced chemiluminescence (ECL) detection system (GE Healthcare) and quantified by ImageJ software.

### RT-PCR

For in-vivo study tissues were collected form 12 animals described as sacrificed group. Tissues were homogenized in 1 ml Invitrogen TRIzol™ Reagent using metal beads combined with high-speed shaking (Tissuelyser Qiagen at 20 Hrtz, for 6 min). Skin samples were crushed with pistol and liquid nitrogen prior to homogenizing step .The chloroform extraction and ethanol precipitation were performed on homogenized tissues to extract RNA. The RNA quality and quantity were assessed and cDNA were synthesized as described. Gene expression was quantified by real-time quantitative PCR using the Roche Universal Probe Library system (Indianapolis, Ind., USA). The primer sets (0.1 μM) were as follows: 1) p16 F:5′-AACTCTTTCGGTCGTACCCC-3′ and R: 5′-TCCTCGCAGTTCGAATCTG -3′ with Custom designed probe: 5′-/56-FAM/AGGTGATGA/ZEN/TGATGGGCAACGTTCAC/3IABkFQ -3′. 2) p21 R: 5′-TTTGCTCCTGTGCGGAAC -3′ and F:5′- TTGCCAGCAGAATAAAAGGTG-3′ with probe #9. Transcript levels were normalized to Beta-glucuronidase (GUSB) as an endogenous control.

### Results

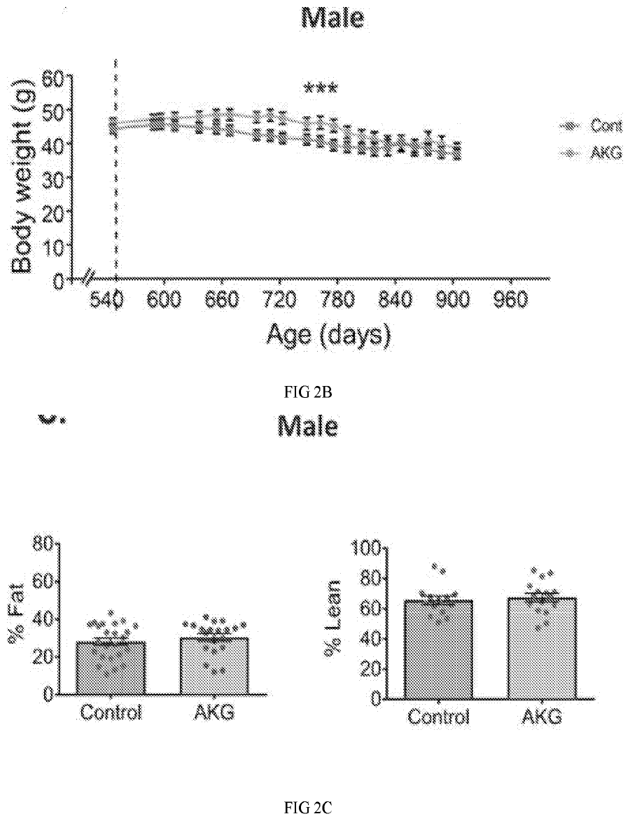

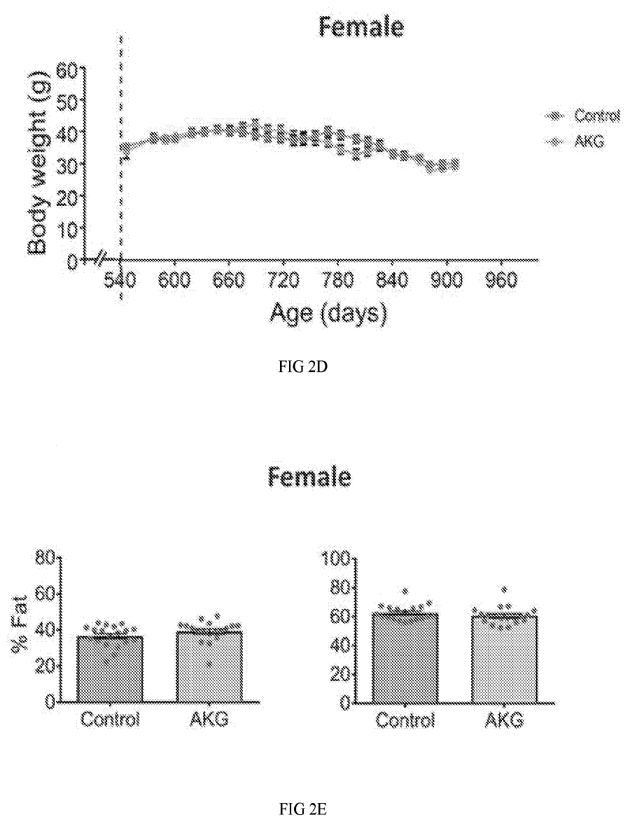

C57Bl/6 mice were fed regular chow until they were started on diet containing CaAKG at 540 days of life. The mice were fed AKG or Standard diet at 18 months of age. The study consisted of two cohorts (n=182 animal total) and a sacrifice group (n=12). Cohort-1 mice were used for all the frailty index measurements and lifespan. Cohort-2 mice were started on diets at the same age for replication of survival, metabolic studies and complementary aging studies. The two cohorts of mice each consisting of 45 females and 45 males (total of 180 animals) were assessed. Here we report that diets supplemented with 2% CaAKG (w/w) increases survival in two independent cohorts of aged mice (FIG. 2A, P=0.032, P=0.022). In the first cohort, median lifespan and survival (age at 90th percentile mortality) were extended by 9.7% and 20% from inception of treatment. Female mice had 19.4% improved survival from inception of CaAKG feeding (FIG. 2B, P=0.02, and Table 3). Although, improved survival for males was not significant (FIG. 2C, P=0.18 and P=0.15), it was extended for 9.4% (FIG. 2C, and Table 3). These findings have been repeated in a second cohort of mice, while some mice remain alive, median lifespan was extended 9.6% in females and 10.4% in males (P=0.037, P=0.15).

TABLE 3
The effect of AKG on lifespan.

| | | Age (days) at 50% | | Age (days) at 90% | | Age (days) at | |
|---|---|---|---|---|---|---|---|
| | | mortality | | mortality | | 100% | |
| | | Cohort-1 | Cohort- | Cohort-1 | Cohort-2 | Cohort-1 | Cohort-2 |
| Female | Control | 876 | 870 | 1015 | 958 | 1016 | — |
| | AKG | 932 | 902 | 1109 | — | 1116 | — |
| | Percentage survival | 16.6% | 9.6% | 19.7% | — | 21% | — |
| | increase from | | | | | | |
| | nception of study | | | | | | |
| | Percentage | 6.3% | 3.7% | 9% | — | 10% | — |
| | survival increase | | | | | | |
| | from birth (AKG [symbol] | | | | | | |
| Male | Control | 955 | 839 | 1068 | — | 1109 | — |
| | AKG | 995 | 870 | 1155 | — | 1173 | — |
| | Percentage survival | 9.6% | 10.4% | 16.4% | — | 11.2% | — |
| | inception of study | | | | | | |
| | [symbol] | | | | | | |
| | Percentage survival | 4.2% | 3.7% | 8% | — | 6% | — |
| | increase in survival | | | | | | |
| | from birth (AKG vs. | | | | | | |
| | [symbol] | | | | | | |
| male & | Control | 938 | 839 | 1019 | — | 1094 | — |
| female | AKG | 977 | 870 | 1116 | — | 1173 | — |
| | Percentage survival | 9.7% | 10.4 | 20.2% | — | 14.2% | — |
| | increase from | | | | | | |
| | inception of study | | | | | | |
| | (AKG vs. Cntrl) | | | | | | |
| | Percentage survival | 4.1% | 3.7% | 9.5% | — | 7.2% | — |
| | increase in survival | | | | | | |
| | from birth (AKG vs. | | | | | | |
| | Cntrl) | | | | | | |

[symbol]

indicates data missing or illegible when filed

In order to assess healthspan, measurements were applied that are based on a clinically relevant frailty index. The frailty index (FI) consists of 31 phenotypes that are indicators of age-associated health deterioration, with each phenotype scored on a 0, 0.5 or 1 scale, based on its severity. Body weight and surface temperature were collected and converted to the same scoring scales (Table 2). All scorings were conducted in a blinded manner.

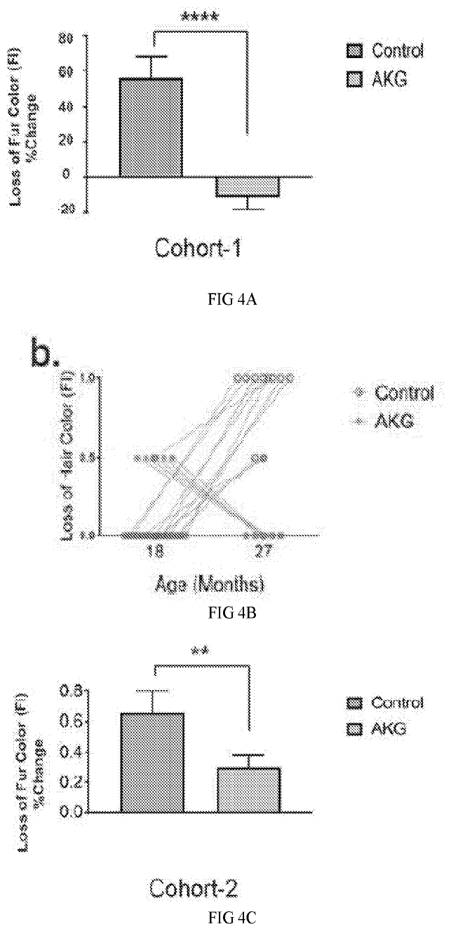

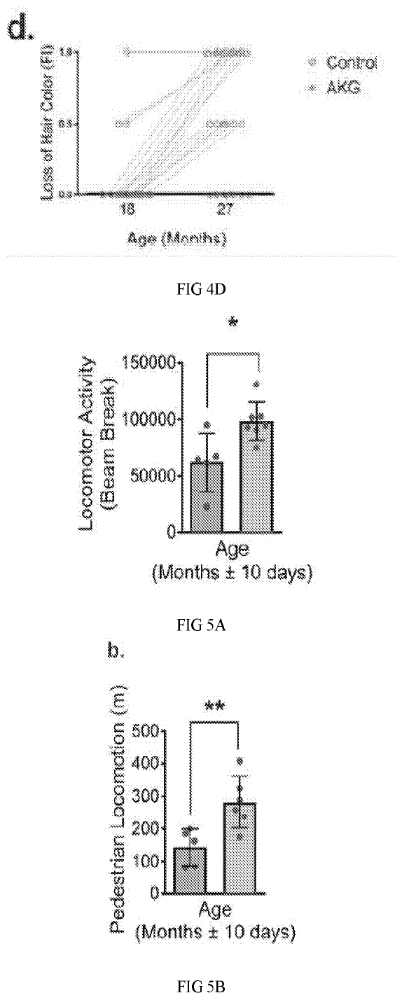

The 31-metrics share many characteristics of the human frailty indices and has been reported to progress similarly with aging in mice and humans. Measurements were repeated approximately every eight weeks, providing us with eight and seven sets of data respectively for male and female groups. To establish a baseline clinical assessment, a dataset was collected right before the start of the treatment at 18th months (FIG. 4A-FIG. 4D). The total frailty score, which is the sum of 31 frailty phenotypes, indicates the state of increased vulnerability to adverse health outcomes, which is comparable to morbidity. In both female and male animals, CaAKG decreases incidence and severity of different aging phenotypes and postpones morbidity (FIG. 4A-FIG. 4B). Male animals start to show significant health improvement after 11 months of CaAKG supplementation which persisted until the last measurement at 33 months of age (P<0.01, P<0.05, FIG. 4A). Females show significant improvements after 9 months of treatment and again at 11 months post treatment (P<0.001, P<0.05, FIG. 4B).

To determine which aging phenotypes have been most affected, individual frailty indicators were compared at different time points (FIG. 4C, and FIG. 4D). CaAKG treatment significantly decreased the severity of multiple aging phenotypes in males, including; grip strength loss, gait disorder, balance loss (vestibular disturbance), grimace (pain assessment), loss of menace reflex, hearing loss, eye discharge, poor coat condition, poor body condition, tail stiffening and abnormal breathing rate (FIG. 4C). In females, piloerection, grimace, alopecia, loss of fur color, poor coat condition, grip strength loss, gait disorder, rectal prolapse and body weight loss were significantly decreased (FIG. 4D).

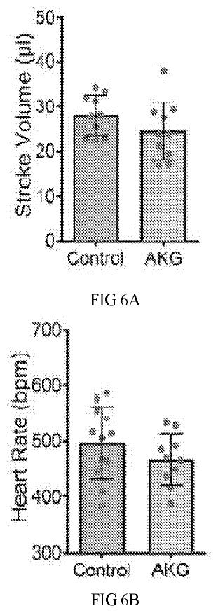

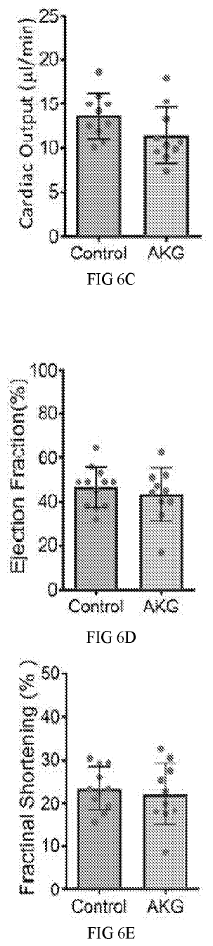

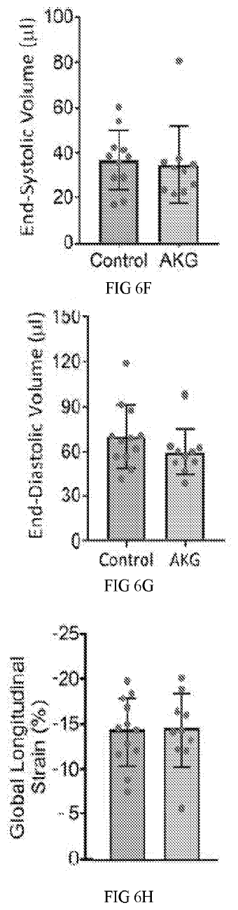

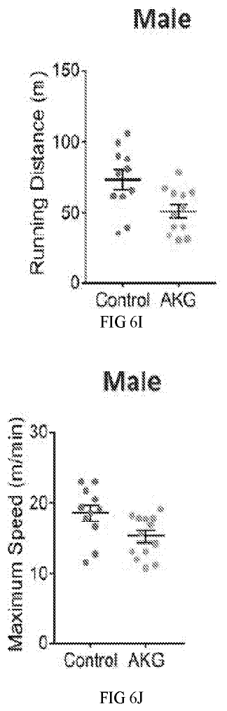

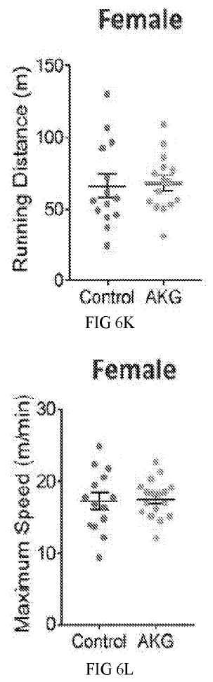

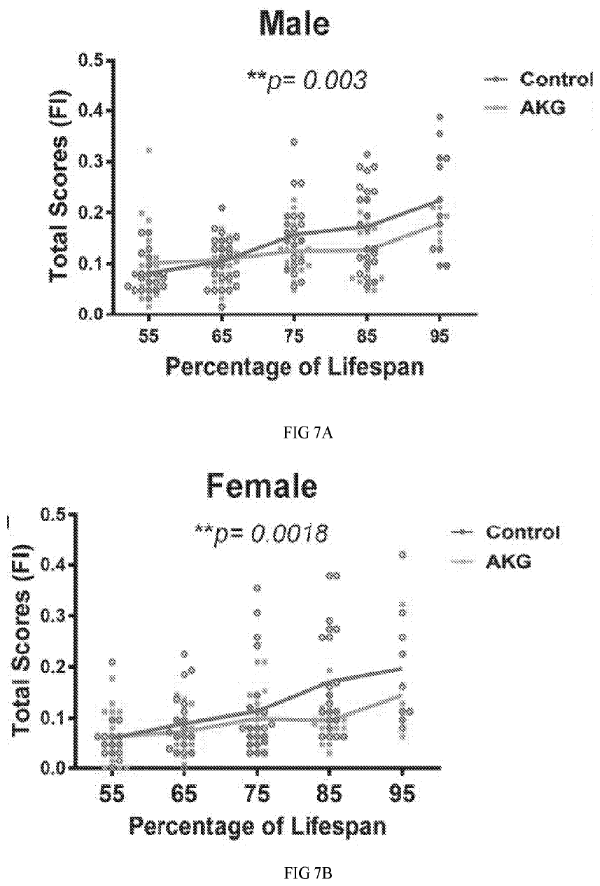

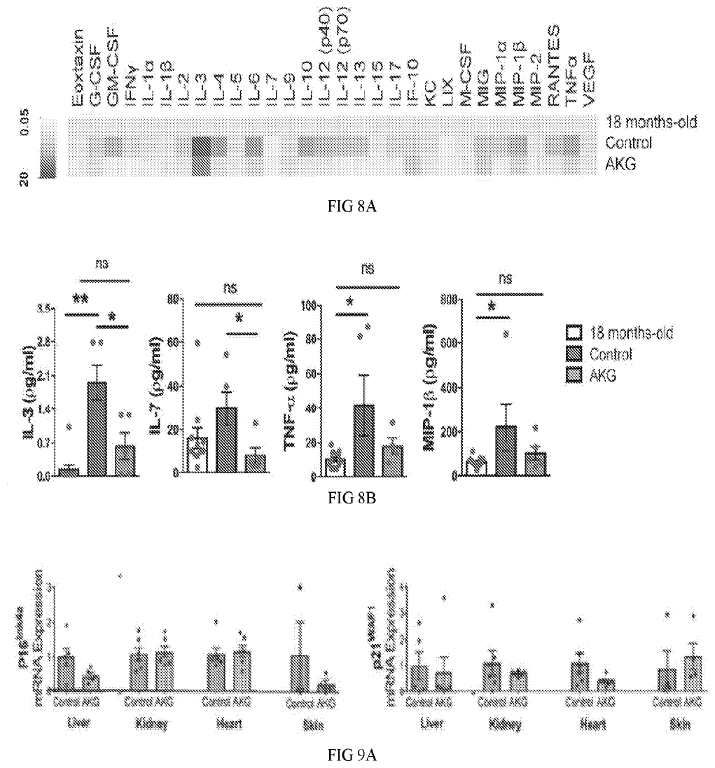

The improvement in health of both sexes was most prominent around the median life of the animal. Among the frailty measures affected by CaAKG, protection from age-related changes in female coat color was particularly prominent. CaAKG treatment could reverse age-dependent hair graying in first cohort, however in the second cohort of females, CaAKG treatment only prevented the hair graying and no reversal effect was detected (FIG. 5A-FIG. 5D). Gait improvement in CaAkG treated females and males close to median life is consistent with increased locomotion activity in metabolic cages (FIG. 6A-FIG. 6B). The levels of oxygen consumption, carbon dioxide production and energy expenditure were significantly lower in the CaAKG treated group. The decrease in carbon dioxide production is preserved later in life (not shown). Advanced age-related weight loss has been reported in old human and rodents, which has been associated with morbidity. Data from the second cohort of mice confirms the age-related weight loss in both sexes and we observed better body weight preservation by AKG in male mice (P value<0.001, FIG. 3B, FIG. 3D). Not all phenotypes were improved, to study the heart and motor function treadmill exhaustion test and heart echocardiography was run. No significant adverse changes with CaAKG treatment were detected (FIG. 8A-FIG. 8L).

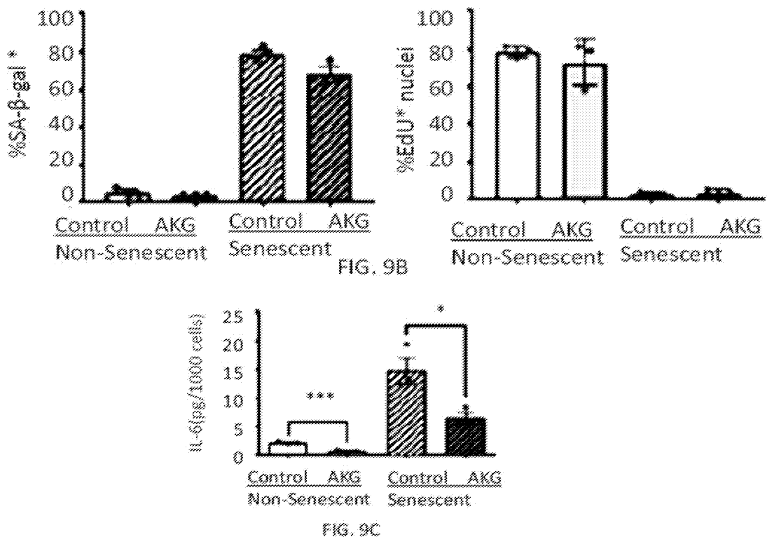

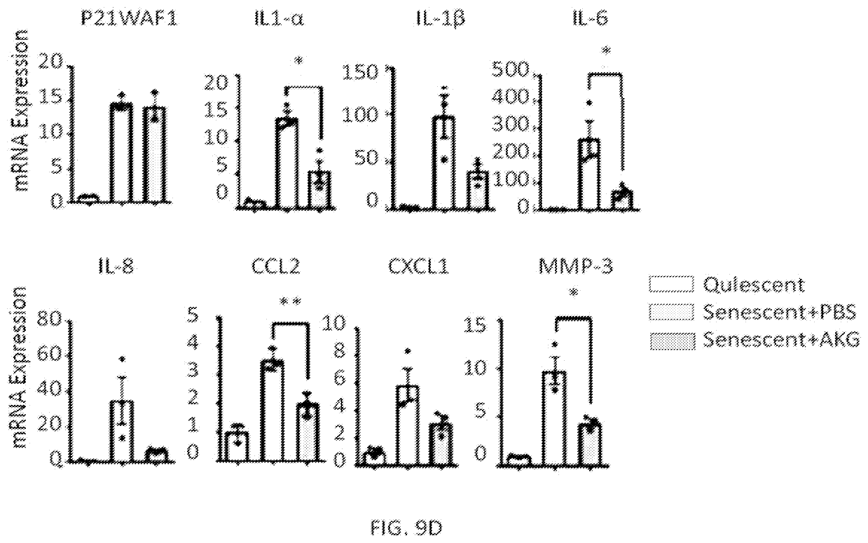

Since the age for onset of age-related phenotypes can be quite heterogeneous in mammals, frailty datasets were plotted not only as a function of chronological time, but also in proportion to the lifespan of each mouse by binning scores within ten percentile (e.g. scores collected between 60% and 70% of the animals' lifespans were binned and plotted at 65%), (FIG. 9A-FIG. 9B). This further aligns assessments with respect to the biologic age of the animal.

AKG treatment as shown, decreases the proportion of life in which the animal is frail and vulnerable to adverse health incomes (determined as the area under the frailty curve and calculated at a 52% reduction for females and 40% for males, FIG. 9A-FIG. 9B, P<0.01). This improvement in healthy days of life is disproportionately larger than the increase in lifespan. Age-associated diseases are accompanied by chronic inflammation, which is generally linked to an age-associated functional decline. Levels of 30 inflammatory cytokines in the serum of aged female mice (29 month old) were measured. In untreated mice, the levels of most cytokines increase; however, CaAKG fed animals were largely refractory to these changes (FIG. 10A). Data shows significant changes specifically for IL-3, IL-7, TNFα and MIP-1β (FIG. 10B).

### Example 2: Effect of AKG on SASP Phenotype

### Cell Culture

IMR-90 fetal lung fibroblasts were obtained from ATCC and were cultured at 37° C. in 3% O₂ and 5% CO₂. Dulbecco's modified Eagle's media (DMEM) supplemented with 10% fetal bovine serum and streptomycin/penicillin were used. Media was changed every 2 days during the experiment. For damage-induced senescence, cells were irradiated with doses of either 0 or 10 Gy of ionizing radiation (IR). Cells were concurrently treated with PBS (control) or 1 mM AKG for 10 days, changing media every 2 days. All assays were performed 10 days post irradiation.

EdU (5-ethynyl-2′-deoxyuridine) staining Proliferation Kit (iFluor 488) ab219801 was used to detect cell proliferation. Cells were stained for the senescence-associated 3-gal (SA-3-gal) marker as described. Non-senescent cells (having undergone fewer than 35 population doublings) were made quiescent by washing with PBS and incubating in DMEM containing 0.2% serum for 4 day. Cultures that had >80% SA-3-gal positive cells and ≤4% EdU positive cells were considered senescent.

### ELISA

Conditioned media were prepared by washing cells 3 times in PBS and incubating them in serum-free DMEM containing penicillin/streptomycin for 24 h. Conditioned media were removed and cells were trypsinized for cell counts. The conditioned media were then centrifuged to remove cellular debris, and supernatants were used for ELISA. IL-6 ELISAs were performed using kits and procedures from R&D (#D06050). The resultant data were normalized to cell number.

### RT PCR

For cell culture experiments, RNA was isolated using ISOLATE II RNA mini kit (Bioline #BIO-52073). RNA quality and quantity were assessed using NanoDrop™ 1000 spectrophotometer measures (Thermo Scientific). Total cDNAs were synthesized from 500 ng of RNA using random primers and iScript RT reagents following the manufacturer's protocol Superscript II (Invitrogen, Carlsbad, USA). Gene expression was measured from cDNA using the Roche Universal Probe Library system (Indianapolis, Ind., USA). All values were normalized to beta-actin.

### Results

Senescent cells accumulate in different tissues of old mice. These cells contribute to age-associated chronic inflammation by acquiring senescence-associated secretory phenotype (SASP) and their removal can extend lifespan. Although trends toward reduced levels of senescent markers were observed in some tissues, no significant changes were seen for senescent markers (FIG. 11A). Cell culture studies were performed to explore possible effects of NaAKG in primary fibroblasts. While no changes in the senescence formation were observed; however, consistent to in-vivo findings seen in FIG. 10A-FIG. 10B, a significant reduction in a range of inflammatory cytokines were detected, indicating that AKG can alter the SASP and reduce inflammation without effecting the formation of senescent cells (FIG. 11B). Specifically, a reduction of IL-1b, IL-6, CCL2 and MMP3 without any changes in β-gal and p21 was also detected (FIG. 11C-FIG. 11D).

### Example 3: Exemplary Oral Formulations

Oral formulations such as tablets may be used for patient treatment or clinical data. Tablets can be sustained release tablets that release the active ingredients over 8-12 hours. In this example, product tablets are 0.500″ round and 0.290″ thick. The formulations may comprise Vitamin A (as retinyl palmitate), Vitamin D3, Calcium (in the form of calcium alpha-ketoglutarate monohydrate), calcium alpha-ketoglutarate monohydrate, isomalt, vegetable wax (Carnauba and/or Rice Bran), stearic acid, magnesium stearate and silica.

Different formulations may be used for men and women. For instance, for men the formulation may comprise: Vitamin A—450 mcg, calcium—95 mg and Calcium Alpha-Ketoglutarate Monohydrate—500 mg. Women formulations may comprise: Vitamin D3—12.5 mcg, calcium—95 mg and Calcium Alpha-Ketoglutarate Monohydrate—500 mg.

### Example 4: Exemplary Patient Experiments or Clinical Trials

The study is an open label, randomized study to obtain safety and biomarker data on Rejuvant dietary supplement products. The study may have four Groups as outlined in the table below. Groups 1-4 are divided by gender and each Group receive the gender-specific investigational product. Groups 1 and 3 take the commercial serving size of two tablets per day of the gender-specific investigational product. Groups 2 and 4 take three tablets per day of the gender-specific investigational product; these Groups provide data at a larger serving size than the recommended dose. All Groups take the investigational product for six to nine months. The participants are assessed at four time points: Study Initiation, 3-months, 6-months and 9-months. At each visit a vitals assessment is conducted (height, weight, blood pressure, pulse), blood chemistries, Hemoglobin A1C, C-Reactive Protein and Uric Acid will be measured. In addition, a blood sample is taken at each visit for metabolite analysis. At visits 1 and 4, a saliva sample is collected for DNA methylation analysis.

The physician exam, blood chemistries, Hemoglobin A1C, C-Reactive Protein, Uric Acid levels and the questionnaire allows for an assessment of the safety of the investigational products. These data are used to assess changes in participant metabolism over the course of the study. The blood chemistry and metabolite data is used to calculate the biological age of the participant by one or more published algorithms. DNA methylation analysis of saliva determines the degree of DNA methylation (the cytosine of the CpG dinucleotide is methylated). The degree of DNA methylation is correlated with aging and is used as another measure of the biological age of the participant.

### Inclusion Criteria

Men 45-65 years
Female 45-65 years

### Exclusion Criteria

Females of child-bearing age
Pregnant females
Lactating females
Diabetics
Congestive Heart Disease
Myocardial Infarction in the previous year
Undergoing treatment for cancer
Morbidly Obese

### Patient Groups in the Clinical Trial

| | | # of | | Dose |
|---|---|---|---|---|
| Group | Test Article | Volunteers | Gender | (# of Tablets) |
| 1 | Rejuvant ® for Men | 20 | Male | 2 |
| 2 | Rejuvant ® for Men | 20 | Male | 3 |
| 3 | Rejuvant ® for Women | 20 | Female | 2 |
| 4 | Rejuvant ® for Women | 20 | Female | 3 |

### Primary Outcome Measures

Primary outcome measures are used to establish the safety of oral administration of the therapeutic composition. Safety and tolerability of single and multiple doses of a composition described herein is measured by adverse events (AEs), physical examinations (PE), vital signs (VS), laboratory safety tests, urinalysis and 12-lead electrocardiograms (ECG). Outcomes measured during site visits include: height, weight, blood pressure and pulse, complete blood ounts, comprehensive metabolic panel, lipid panel and blood measures such as hemoglobin A1C, c-reactive protein levels and uric acid.

Complete Blood Count may include: WBC (White Blood Cell Count), Hemoglobin, Hematocrit, Platelet Count/L, MCV (Mean Corpuscular Volume), MCH (Mean Corpuscular Hemoglobin), MCHC (Mean Corpuscular Hemoglobin Concentration), RBC (Red Blood Cell Count), RDW-CV (Red Cell Distribution Width), MPV (Mean Platelet Volume), Neutrophils, Neutrophil %, Lymphocyte Count, Lymphocyte %, Monocyte Count, Monocyte %, Eosinophils, Eosinophil %, Basophils, Basophil %.

Comprehensive Metabolic Panel may include measuring: Sodium, Potassium, Chloride, Carbon Dioxide (CO₂), Urea Nitrogen (BUN), Creatinine, Glucose, Calcium, AST (Aspartate Aminotransferase), ALT (Alanine Aminotransferase), Alk Phos (Alkaline Phosphatase), Bilirubin, Total, Alkaline Phosphatase, Albumin, Protein, Total, Anion Gap, Glomerular Filtration Rate (eGFR), MDRD Estimate.

Lipid Panel may include: Total Cholesterol, LDL (Low Density Lipoprotein) Cholesterol, HDL (High Density Lipoprotein) Cholesterol, Triglyceride,

### Secondary Outcome Measures

The secondary outcome is to observe changes in blood chemistries, metabolites and the amount of DNA methylation by the oral administration of Rejuvant products per dosing described. The secondary outcome measures may include Metabolic Screen (from blood samples), DNA Methylation Assay (from saliva samples).

### Example Questionnaire

1. How do you feel today?
2. How would you describe your daily activity?
3. How would you rate your physical energy level?
4. How would you rate your cardiovascular endurance level?
5. How would you describe your ability to focus?
6. How would you rate your level of memory recall?
7. How would you describe your quality of sleep?
8. How would you rate the youthfulness of your hair?
9. How many minutes per week do you exercise including light exercise?
10. Is there any other relevant information about the product you'd like to share?

An example clinical trial flow is described:

| | | |
|---|---|---|
| Prior to | N = 50 Male Participants | N = 50 Female Participants |
| Enrollment | Screen potential participants by | Screen potential participants by |
| | inclusion and exclusion criteria | inclusion and exclusion criteria |
| Enrollment | Obtain history | Obtain history |
| | Obtain informed consent | Obtain informed consent |
| Randomize | Group 1 N = 25 | Group 3 N = 25 |
| | Group 2 N = 25 | Group 4 N = 25 |

| | |
|---|---|
| Visit 1 | Perform Baseline Assessments: |
| | Physical exam, blood chemistries, obtain blood sample for Nightingale, obtain |
| | sample for DNA methylation analysis, distribute 3 month supply of |
| | investigational product |
| Visit 2 | Physical exam, blood chemistries, obtain blood sample for Nightingale, |
| | questionnaire completed, distribute 3 month supply of investigational product |
| Visit 3 | Physical exam, blood chemistries, obtain blood sample for Nightingale, |
| | questionnaire completed, distribute 3 month supply of investigational product |
| Visit 4 | Physical exam, blood chemistries, obtain blood sample for Nightingale, |
| | questionnaire completed, obtain sample for DNA methylation analysis |
| Study | All data transferred for analysis |
| Closeout | |

### Example 5: Exemplary Patient Experiments or Clinical Trials

The study is placebo-controlled, randomized study to obtain safety and biomarker data on Rejuvant dietary supplement products. The study has four Groups as outlined in the table below. Groups 1-4 are divided by gender and each Group receive the gender-specific investigational product or a placebo. Groups 1 and 3 take the commercial serving size of two tablets per day of the gender-specific investigational product. Groups 2 and 4 will take two placebos per day. All Groups take the tablets for six to nine months. The participants are assessed at four time points: Study Initiation, 3-months, 6-months and 9-months. At each visit a vitals assessment is conducted (height, weight, blood pressure, pulse), blood chemistries, Hemoglobin A1C, C-Reactive Protein and Uric Acid will be measured. In addition, a blood sample is taken at each visit for metabolite analysis. At visits 1 and 4, a saliva sample is collected for DNA methylation analysis.

The physician exam, blood chemistries, Hemoglobin A1C, C-Reactive Protein, Uric Acid levels and the questionnaire allows for an assessment of the safety of the investigational products. These data is used to assess changes in participant metabolism over the course of the study. The blood chemistry and metabolite data is used to calculate the biological age of the participant by one or more published algorithms. DNA methylation analysis of saliva determines the degree of DNA methylation (the cytosine of the CpG dinucleotide is methylated). The degree of DNA methylation is correlated with aging and is used as another measure of the biological age of the participant.

### Inclusion Criteria

Men 45-65 years
Female 45-65 years

### Exclusion Criteria

Females of child-bearing age
Pregnant females
Lactating females
Diabetics
Congestive Heart Disease
Myocardial Infarction in the previous year
Undergoing treatment for cancer
Morbidly Obese

### Patient Groups in the Clinical Trial

| | | # of | | Dose |
|---|---|---|---|---|
| Group | Test Article | Volunteers | Gender | (# of Tablets) |
| 1 | Rejuvant ® for Men | 20 | Male | 2 |
| 2 | Placebo for Men | 20 | Male | 2 |
| 3 | Rejuvant ® for Women | 20 | Female | 2 |
| 4 | Placebo for Women | 20 | Female | 2 |

### Primary Outcome Measures

Primary outcome measures are used to establish the safety of oral administration of the therapeutic composition. Safety and tolerability of single and multiple doses of a composition described herein is measured by adverse events (AEs), physical examinations (PE), vital signs (VS), laboratory safety tests, urinalysis and 12-lead electrocardiograms (ECG). Outcomes measured during site visits include: height, weight, blood pressure and pulse, complete blood counts, comprehensive metabolic panel, lipid panel and blood measures such as hemoglobin A1C, c-reactive protein levels and uric acid.

Complete Blood Count may include: WBC (White Blood Cell Count), Hemoglobin, Hematocrit, Platelet Count /L, MCV (Mean Corpuscular Volume), MCH (Mean Corpuscular Hemoglobin), MCHC (Mean Corpuscular Hemoglobin Concentration), RBC (Red Blood Cell Count), RDW-CV (Red Cell Distribution Width), MPV (Mean Platelet Volume), Neutrophils, Neutrophil %, Lymphocyte Count, Lymphocyte %, Monocyte Count, Monocyte %, Eosinophils, Eosinophil %, Basophils, Basophil%.

Comprehensive Metabolic Panel may include measuring: Sodium, Potassium, Chloride, Carbon Dioxide (CO₂), Urea Nitrogen (BUN), Creatinine, Glucose, Calcium, AST (Aspartate Aminotransferase), ALT (Alanine Aminotransferase), Alk Phos (Alkaline Phosphatase), Bilirubin, Total, Alkaline Phosphatase, Albumin, Protein, Total, Anion Gap, Glomerular Filtration Rate (eGFR), MDRD Estimate.

Lipid Panel may include: Total Cholesterol, LDL (Low Density Lipoprotein) Cholesterol, HDL (High Density Lipoprotein) Cholesterol, Triglyceride,

### Secondary Outcome Measures

The secondary outcome are to observe changes in blood chemistries, metabolites and the amount of DNA methylation by the oral administration of Rejuvant products per dosing described. The secondary outcome measures may include Metabolic Screen (from blood samples), DNA Methylation Assay (from saliva samples).

### Example Questionnaire

1. How do you feel today?
2. How would you describe your daily activity?
3. How would you rate your physical energy level?
4. How would you rate your cardiovascular endurance level?
5. How would you describe your ability to focus?
6. How would you rate your level of memory recall?
7. How would you describe your quality of sleep?
8. How would you rate the youthfulness of your hair?
9. How many minutes per week do you exercise including light exercise?
10. Is there any other relevant information about the product you'd like to share?

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-46. (canceled)

47. A method for increasing mobility in a host animal, the method comprising administering to the host animal a composition comprising a therapeutically effect amount of an alpha-ketoglutarate.

48. The method of claim 47 for increasing exercise endurance in the host animal.

49. The method of claim 47 for increasing exercise efficiency, metabolism, or both, in the host animal.

50. The method of claim 47 for improving gait in the host animal.

51. The method of claim 47 for improving locomotion in the host animal.

52. The method of claim 47 for improving balance in the host animal.

53. A method for treating sarcopenia in a host animal, the method comprising administering to the host animal a composition comprising a therapeutically effect amount of an alpha-ketoglutarate.

54. The method of claim 47 for increasing collagen synthesis in the host animal.

55. The method of claim 47 wherein increasing mobility includes altering secretion of at least one SASP factor.

56. The method of 47 wherein increasing mobility includes reducing secretion of at least one SASP factor.

57. The method of claim 55 wherein the one or more SASP factors are selected from the group consisting of interleukins (ILs), chemokines, inflammatory factors, growth factors, proteases, extracellular matrix components, and combinations thereof.

58. The method of claim 55 wherein the one or more SASP factors are selected from the group consisting of IL-1β, IL-3, IL-6, IL-7, MIP-1β, TNF-α, CCL2, MMP3, and combinations thereof.

59. The method of claim 55 wherein at least one SASP factor is p16^{INK4a}.

60. The method of claim 55 wherein at least one SASP factor is p21^{WAF1}.

61. The method of claim 55 wherein altering secretion of at least one SASP factor does not clinically affect the formation of senescent cells in the host animal.

62. The method of claim 55 wherein altering secretion of at least one SASP factor does not clinically affect the number of senescent cells in the host animal.

63. The method of claim 47 wherein the composition is administered twice daily.

64. The method of claim 47 wherein the composition is administered orally.

65. The method of claim 47 wherein the host animal has at least a 6-fold lower level of plasma AKG compared to a control animal, where the control animal is at least 2 years younger than the host animal.

66. The method of claim 47 wherein the therapeutically effective amount of the alpha-ketoglutarate is in the range from about 350 mg to about 2000 mg of calcium alpha-ketoglutarate or calcium alpha-ketoglutarate monohydrate, or a combination thereof.


SEQUENCE LISTING (5 sequences)

SEQ ID NO: 1 - DNA, 20 bp, Homo sapiens
aactctttcggtcgtacccc

SEQ ID NO: 2 - DNA, 19 bp, Homo sapiens
tcctcgcagttcgaatctg

SEQ ID NO: 3 - DNA, 29 bp, Artificial Sequence
  Nucleic acid
  Nucleic acid (1)..(1): isomer derivative of fluorescein attachment for oligonucleotides
  misc_feature (1)..(1): n is a, c, g, or t
  Nucleic acid (11)..(11): internal quencher for TaqMan and qPCR probes
  misc_feature (11)..(11): n is a, c, g, or t
  Nucleic acid (29)..(29): Iowa Black quencher ideal for use with fluorescein
  misc_feature (29)..(29): n is a, c, g, or t
naggtgatgantgatgggcaacgttcacn

SEQ ID NO: 4 - DNA, 18 bp, Homo sapiens
tttgctcctgtgcggaac

SEQ ID NO: 5 - DNA, 21 bp, Homo sapiens
ttgccagcagaataaaaggtg